SEQUENCE LISTING (29 sequences)

SEQ ID NO: 1 - protein, 303 aa, Candida utilis
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 2 - DNA, 912 bp, Candida utilis
atgtcaacaacgctctcatcatccacctacggcaaggacaacgtcaagttcctcaaggtcaagaaggacccgcaaaacccaaagaagcaggaggttatggaggccaccgtcacgtgtctgcttgaaggtgggttcgacacctcgtacacggaggctgacaactcgtccatcgtgccaacagacaccgtgaagaacaccattctcgtgttggcaaagaccacggagatttggccaattgagagatttgcagccaagctggccacgcactttgttgagaagtactcgcacgtctctggcgtctccgtcaagattgtccaggacagatgggtcaagtacgccgttgatggcaagccacacgaccactcttttatccacgaaggtggtgagaagagaatcactgacctgtactacaagagatccggtgattacaagctgtcgtctgccatcaaggacttgacggtgctgaagtccaccggctcgatgttctacggctacaacaagtgtgacttcaccaccttgcaaccaacaactgacagaatcttgtccaccgacgtcgatgccacctgggtttgggataacaagaagattggctctgtctacgacatcgccaaggctgcagacaagggaatctttgacaacgtttacaaccaggctagagagatcaccttgaccacctttgctctcgagaactctccatctgtgcaggccacgatgttcaacatggctactcagatcttggaaaaggcatgctctgtctactcggtttcatacgccttgccaaacaagcactacttcctcattgacttgaaatggaaaggtttggagaacgacaacgagttgttctacccatctccacatccaaatgggttgatcaagtgtactgttgtccgtaaggagaagaccaagttgtag

SEQ ID NO: 3 - DNA, 37 bp, Glycine max
agatatacatatggcgcagcaggaagtggttgaaggc

SEQ ID NO: 4 - DNA, 55 bp, Glycine max
agatatacatatgcaccatcaccatcaccatgcgcagcaggaagtggttgaaggc

SEQ ID NO: 5 - DNA, 31 bp, Glycine max
tatggatccttacagtttgctccacagacgg

SEQ ID NO: 6 - DNA, 34 bp, Arthrobacter globiformis
agatatacatatgaccgcgaccgcggaaaccagc

SEQ ID NO: 7 - DNA, 52 bp, Arthrobacter globiformis
agatatacatatgcatcaccatcaccatcacaccgcgaccgcggaaaccagc

SEQ ID NO: 8 - DNA, 32 bp, Arthrobacter globiformis
aatggatccttagcaaaagcccgcaatgttgc

SEQ ID NO: 9 - DNA, 34 bp, Phaseolus vulgaris
agatatacatatggcgcaggaagtggttgaaggc

SEQ ID NO: 10 - DNA, 52 bp, Phaseolus vulgaris
agatatacatatgcatcaccatcaccatcacgcgcaggaagtggttgaaggc

SEQ ID NO: 11 - DNA, 31 bp, Phaseolus vulgaris
attggatccttacagtttgctccacacacgg

SEQ ID NO: 12 - DNA, 34 bp, Candida utilis
agatatacatatgagcaccaccctgagcagcagc

SEQ ID NO: 13 - DNA, 52 bp, Candida utilis
agatatacatatgcatcaccatcaccatcacagcaccaccctgagcagcagc

SEQ ID NO: 14 - DNA, 37 bp, Candida utilis
ataggatccttacagtttggttttttctttacgcacc

SEQ ID NO: 15 - DNA, 31 bp, Pseudomonas aeruginosa
agatatacatatgccgaaaagcagcgcggcg

SEQ ID NO: 16 - DNA, 53 bp, Pseudomonas aeruginosa
agatatacatatgcatcaccatcaccatcacccgaaaagcagcgcggcggaac

SEQ ID NO: 17 - DNA, 31 bp, Pseudomonas aeruginosa
taaggatccttacgccgcaaccggcggcggc

SEQ ID NO: 18 - DNA, 20 bp, T7 bacteriophage
taatacgactcactataggg

SEQ ID NO: 19 - DNA, 18 bp, T7 bacteriophage
ctagttattgctcagcgg

SEQ ID NO: 20 - DNA, 930 bp, Glycine max
atggctcagcaggaagtggtagaagggttcaagttcgaacagaggcacgggaaagaacgcgtgagagtagcgcgcgtgtggaagacgaggcaggggcagcacttcattgtggagtggcgcgtggggatcactctcttttcggattgcgtcaactcgtacctccgcgatgacaactctgaaatcgttgctactgataccatgaaaaacaccgtgtatgcaaaagcaaaggaatgctctgacatactttctgccgaggagtttgctattctgcttgctaagcactttgtatcattttaccagaaggttactggtgctattgtgaatattgtggaaaaaccatgggagcgtgtcactgtggatggtcaacctcatgaacatggtttcaaacttgggtctgagaagcatacaacagaggcgatagtacaaaagtctggttcacttcagttgacttctggtattgaaggattgtcagtgttgaagacaacccagtctggttttgtgaatttcataagagacaagtacacagcacttcctgatacccgtgaaaggatggtagcaacagaagtaaccgcactgtggaggtattcgtatgaatcgctgtatagcctccctcagaagccgctttactttacagaaaagtatcaggaagtgaaaaaagttctggctgacactttttttggcccaccaaaagggggagtctatagcccatctgttcaaaacacactctacctgatggcaaaggccacactgaacagatttcctgacatagcttatgtcagtctaaagttgccaaatcttcatttcatacctgtcaatatctcaaaccaggatggccctattgtgaagtttgaggatgatgtgtacttgccaacggatgagccacatgggtcaattcaagctagtttgagccgcctttggtcaaagctgtag

SEQ ID NO: 21 - DNA, 927 bp, Phaseolus vulgaris
atggcgcaggaagttgtggaggggttcaagtttgagcagaggcacgggaaagagcgcgtcagagttgcgcgcgtgtggaggacgccgcagggtcgccacttcgttgtggagtggcgcgtaggtattactctcttctctgattgcgtcaactcgtatctccgcgatgataactctgacatcgttgccactgacaccatgaaaaacacggtgtatgcaaaagcaaaggaatgctcggatatactttctgtcgaggactttgctattctacttgccaagcactttgtatcattttacaagaaggttactggtgctattgtgaatatcgtggaaaaaccatgggagcgtgtcattgtggatggtcaacctcatcaacatggtttcacacttgggtctgagaagcatacaacagaggcaatagtacagaagtctggttcactacaattgacttctggtattgaaggattgtcagtgttgaagacaacccagtctggttttgagaatttcattagaaacaagtacacagcacttccagatacccgcgaaaggattttggcaacagaagtaactgctctgtggaggtattcgtacgaatctctatacaacctccctcagaagccactatacttcacagacaagtatctggaagtgaaaaaagttctggctgacacattttttgggccaccaaacaggggagtctatagcccatctgttcaaaacacactctaccttatggcaaaggccacactgaacagatttcctgacattgcttatgtccatctaaagatgccaaatcttcatttcttaccagtcaacatctcaagcaaggatggtccaattgtgaagtttgaggatgatgtttatttaccaacggacgagcctcatggctcaattgaagcaagcttgagccgggtttggtcaaagctgtag

SEQ ID NO: 22 - DNA, 909 bp, Arthrobacter globiformis
atgactgccaccgcagaaacctcaaccggcaccaaggtcgtgctcggacagaaccagtacggcaaggccgaagtccgcctcgtcaaggtcacgcgcaataccgcccggcacgagatccaggacctgaatgtcacctcgcagctgcgcggcgacttcgaggccgcacacaccgccggcgacaacgcgcacgtggtcgccaccgacacgcagaagaacaccgtctacgccttcgcccgcgacggcttcgccaccaccgaggagttcctgctccggctgggcaaacacttcaccgagggcttcgactgggtaaccggcgggcgctgggcggcgcagcagttcttctgggaccgcatcaacgaccacgaccacgccttctcccggaacaagagcgaggtccgcaccgccgtgctcgagatctcgggcagcgagcaggccatcgtcgccgggatcgagggcctgacggtcctgaagtccaccggttcggaattccacggcttcccgcgggacaagtacaccaccctgcaggaaaccaccgaccgtatcctcgccacggatgtcagcgcccgctggcgctacaacaccgtcgaggttgacttcgacgccgtctacgcgagcgtccgcgggctgctgctcaaggccttcgccgagacccactcgctggccctgcagcagaccatgtatgagatgggccgggccgtcatcgagacgcacccggaaatcgacgaaatcaagatgtccctgccgaacaagcaccatttcctggtggacctgcagcccttcggacaggacaacccgaatgaggtgttctacgccgccgaccgtccctacggactgatcgaagccaccatccagcgcgagggctcgcgcgccgaccacccgatctggtcgaacatcgccggattctgctag

SEQ ID NO: 23 - DNA, 912 bp, Candida utilis; identical to SEQ ID NO: 2

SEQ ID NO: 24 - DNA, 1485 bp, Pseudomonas aeruginosa
atgcccaagtcatccgccgccgaacaatccggcgagagttcgacccagaccctgtccctgctcgacgagatcatcgccaagggccgcatggcccacgacgacagccagcaggactatgcccgcgacatgctcgcggaattcgccacccaggtcctcgacgagggcatggccgtcgacaaggacaccgtggcgatgatcaacgaccgcatcagccagatcgatgcgctgatcagcgaccagctcaaccagatcatccaccaccccgagttgcagaagctggaagcctcctggcgcggcctgcaccaactggtgagcaacaccgagaccagcgcgcggctcaagctgcgcctgctgaacgtcggcaagaacgaactgcagaacgacctggagaaggcggtcgagttcgaccagagcgcactgttcaagaagatctacgaagaggaatacggcaccttcggcggacagcccttcagcctgctgatcggcgacttcaccttcggtcgccatccgcaggacatcggcctgctggagaagctgtcgaacgtcgccgcggctgcccacgcgccgttcatcgccgccgccagcccacgcctgttcgacatgaacagcttcaccgaactggccgtgccgcgcgacctgaccaagatcttcgagagcctggagctgatcaagtggcgcgccttccgcgagagcgaggactcgcgctacgtgtcgctggtgctgccgaacttcctcctgcgcctgccctacggcccggagacgcgcccggtggaaggcatgaactatgtcgaggacgtcaacggcaccgaccactccaagtacctctggggcaacgccgcctgggtcctggcgcagcgcatcaccgaggccttcgccaagtacggctggtgcgcggcgatccgcggcgcggaaggcggcggcgcggtcgagggcctgccggcgcacacgttccgcaccagctccggcgacctgtcgctgaagtgcccgaccgaggtggcgatcaccgaccgccgcgagaaggaactcaacgatctcggcttcatttccctgtgccacaagaagaacagcgacgtggcggtgttcttcggcggccagaccaccaacaaggccaggctctacaacaccaacgaggccaacgccaacgcgcgcctgtcggcgatgctgccgtacgtgctggcggcatcgcgcttcgcccactacctgaaggtgatcatgcgcgacaaggtcggcagcttcatgacccgcgacaacgtgcagacctacctgaacaactggatcgccgactacgtgctgatcaacgacaacgcaccgcaggaaatcaaggcgcagtacccgctgcgcgaggcgcgggtggatgtcagcgaggtggccggcaaaccgggggcctaccgcgccacggtgttcctccggccgcacttccagctcgaggaactcagcgcgtcgatccgcctggtcgccaacctgccgccgccggtagcggcgtga

SEQ ID NO: 25 - DNA, 930 bp, Artificial Sequence
  E. Coli optimized uricase
atggcgcagcaggaagtggttgaaggctttaaatttgaacagcgccatggcaaagaacgtgtgcgtgtggcgcgtgtgtggaaaacccgtcagggccagcattttattgtggaatggcgtgttggcattaccctgtttagcgattgcgtgaacagctatctgcgtgatgataacagcgaaattgtggcgaccgataccatgaaaaacaccgtgtatgcgaaagcgaaagaatgcagcgatattctgagcgcggaagaatttgcgattctgctggccaaacattttgtgagcttctaccagaaagtgaccggcgcgattgtgaacattgtggaaaaaccgtgggaacgtgtgaccgtggatggccagccgcatgaacatggctttaaactgggcagcgaaaaacataccaccgaagcgattgtgcagaaaagcggcagcctgcagctgaccagcggcattgaaggcctgagcgtgctgaaaaccacccagagcggctttgtgaactttatccgcgataaatataccgcgctgccggatacccgcgaacgtatggtggcgaccgaagtgaccgcgctgtggcgttatagctatgaaagcctgtatagcctgccgcagaaaccgctgtatttcaccgaaaaatatcaggaagtgaaaaaagttctggccgataccttttttggtccgccgaaaggcggcgtgtatagcccgagcgtgcagaacaccctgtatctgatggcgaaagcgaccctgaaccgttttccggatattgcgtatgtgagcctgaaactgccgaacctgcattttattccggtgaacatcagcaaccaggatggcccgattgtgaaatttgaagatgatgtgtatctgccgaccgatgaaccgcatggcagcattcaggcgagcctgagccgtctgtggagcaaactgtaa

SEQ ID NO: 26 - DNA, 927 bp, Artificial Sequence
  E. Coli optimized uricase
atggcgcaggaagtggttgaaggctttaaatttgaacagcgccatggcaaagaacgtgtgcgtgtggcgcgtgtttggcgtaccccgcagggccgtcattttgtggtggaatggcgtgttggcattaccctgtttagcgattgcgtgaacagctatctgcgtgatgataacagcgatattgtggcgaccgataccatgaaaaacaccgtgtatgcgaaagcgaaagaatgcagcgatattctgagcgtggaagattttgcgattctgctggccaaacattttgtgagcttctataaaaaagtgaccggcgcgattgtgaacattgtggaaaaaccgtgggaacgtgtgattgtggatggccagccgcatcagcatggctttaccctgggcagcgaaaaacataccaccgaagcgattgtgcagaaaagcggcagcctgcagctgaccagcggcattgaaggcctgagcgtgctgaaaaccacccagagcggctttgaaaactttatccgcaacaaatataccgcgctgccggatacccgcgaacgtattctggccaccgaagtgaccgcgctgtggcgttatagctatgaaagcctgtataacctgccgcagaaaccgctgtatttcaccgataaatatctggaagtgaaaaaagtgctggccgataccttttttggtccgccgaaccgtggcgtgtatagcccgagcgtgcagaacaccctgtatctgatggcgaaagcgaccctgaaccgttttccggatattgcgtatgtgcatctgaaaatgccgaacctgcattttctgccggtgaacattagcagcaaagatggcccgattgtgaaatttgaagatgatgtgtatctgccgaccgatgaaccgcatggcagcattgaagcgagcctgagccgtgtgtggagcaaactgtaa

SEQ ID NO: 27 - DNA, 909 bp, Artificial Sequence
  E. Coli optimized uricase
atgaccgcgaccgcggaaaccagcaccggcaccaaagtggtgctgggccagaaccagtatggcaaagcggaagtgcgtctggtgaaagtgacccgtaacaccgcgcgtcatgaaattcaggatctgaacgtgaccagccagctgcgtggcgattttgaagcggcgcataccgcgggtgataacgcgcatgtggtggcgaccgatacccagaaaaacaccgtgtatgcgtttgcgcgtgatggctttgcgaccaccgaagaatttctgctgcgtctgggcaaacactttaccgaaggctttgattgggtgaccggcggtcgttgggcggcgcagcagtttttttgggatcgcatcaacgatcatgatcatgcgtttagccgtaacaaaagcgaagtgcgtaccgcggtgctggaaattagcggcagcgaacaggcgattgtggcgggcattgaaggcctgaccgtgctgaaaagcaccggcagcgaatttcatggctttccgcgcgataaatataccaccctgcaggaaaccaccgatcgtattctggccaccgatgtgagcgcgcgttggcgttataacaccgtggaagtggattttgatgcggtgtatgcgagcgtgcgtggcctgctgctgaaagcgtttgcggaaacccatagcctggccctgcagcagaccatgtatgaaatgggccgtgcggtgattgaaacccatccggaaatcgatgaaatcaaaatgagcctgccgaacaaacatcattttctggtggatctgcagccgtttggccaggataatccgaacgaagtgttttatgcggcggatcgtccgtatggcctgattgaagcgaccattcagcgtgaaggcagccgtgcggatcatccgatttggagcaacattgcgggcttttgctaa

SEQ ID NO: 28 - DNA, 912 bp, Artificial Sequence
  E. Coli optimized uricase
atgagcaccaccctgagcagcagcacctatggcaaagataacgtgaaattcctgaaagtgaaaaaagatccgcagaatccgaaaaaacaagaagtgatggaagcgaccgtgacctgcctgctggaaggcggctttgataccagctataccgaagcggataacagcagcattgtgccgaccgataccgtgaaaaacaccattctggtgctggccaaaaccaccgaaatttggccgattgaacgttttgcggcgaaactggccacccatttcgtggaaaaatattctcatgtgagcggcgtgtctgtgaaaattgtgcaggatcgctgggtgaaatatgcggtggatggcaaaccgcatgatcatagctttattcatgaaggcggcgaaaaacgtattaccgatctgtattataaacgcagcggcgattataaactgagcagcgcgattaaagatctgaccgtgctgaaaagcaccggcagcatgttttatggctataacaaatgcgattttaccaccctgcagccgaccaccgatcgtattctgagcaccgatgtggatgcgacctgggtgtgggataacaaaaaaatcggcagcgtgtatgacattgcgaaagcggcggataaaggcattttcgataacgtgtataaccaggcgcgtgaaattaccctgaccacctttgcgctggaaaacagcccgagcgtgcaggcgaccatgtttaacatggcgacccagattctggaaaaagcgtgtagcgtgtatagcgtgagctatgcgctgccgaacaaacattactttctgatcgatctgaaatggaaaggcctggaaaacgataacgaactgttttatccgagcccgcatccgaacggcctgattaaatgcaccgtggtgcgtaaagaaaaaaccaaactgtaa

SEQ ID NO: 29 - DNA, 1485 bp, Artificial Sequence
  E. Coli optimized uricase
atgccgaaaagcagcgcggcggaacagagcggtgaaagcagcacccagaccctgagcctgctggatgaaattattgcgaaaggccgtatggcgcatgatgatagccagcaggattatgcgcgtgatatgctggccgaatttgcgacccaggtgctggatgaaggcatggcggtggataaagataccgtggcgatgattaacgatcgtatcagccagattgatgcgctgattagcgatcagctgaaccagattattcatcatccggaactgcagaaactggaagcgagctggcgtggcctgcatcagctggtgagcaacaccgaaaccagcgcgcgtctgaaactgcgtctgctgaacgtgggcaaaaacgaactgcagaacgatctggaaaaagcggtggaatttgatcagagcgcgctgttcaaaaaaatctacgaagaagaatacggcacctttggcggccagccgttcagcctgctgattggcgattttacctttggccgtcatccgcaggatattggcctgctggaaaaactgagcaacgtggcggcggccgcacatgcaccgtttattgcggcggcgagcccgcgtctgtttgatatgaacagctttaccgaactggccgtgccgcgtgatctgaccaaaattttcgaaagcctggaactgattaaatggcgtgcgtttcgtgaaagcgaagatagccgttatgtgagcctggtgctgccgaactttctgctgcgtctgccgtatggcccggaaacccgtccggtggaaggcatgaactatgtggaagatgtgaacggcaccgatcatagcaaatatctgtggggcaacgcggcgtgggttctggcccagcgtattaccgaagcgtttgcgaaatatggctggtgcgcggcgattcgtggtgcggaaggcggtggtgcggttgaaggtctgccggcgcatacctttcgtaccagcagcggcgatctgagcctgaaatgcccgaccgaagtggcgattaccgatcgtcgtgaaaaagaactgaacgatctgggctttattagcctgtgccacaaaaaaaacagcgatgtggcggtgttttttggcggtcagaccaccaacaaagcgcgtctgtataacaccaacgaagcgaacgcgaacgcgcgtctgagcgcgatgctgccgtatgttctggccgcgagccgttttgcgcattatctgaaagtgatcatgcgtgataaagtgggcagctttatgacccgtgataacgtgcagacctatctgaacaactggattgcggattatgtgctgattaacgataacgcgccgcaggaaatcaaagcgcagtatccgctgcgtgaagcgcgtgtggatgtgagcgaagtggcgggcaaaccgggtgcgtatcgtgcgaccgtgtttctgcgtccgcattttcagctggaagaactgagcgcgagcattcgtctggtggcgaatctgccgccgccggttgcggcgtaa

(19) United States
(12) Patent Application Publication
(10) Pub. No.: US 2011/0171268 A1
(43) Pub. Date: Jul. 14, 2011

(54) URICASE COMPOSITIONS AND METHODS OF USE

(75) Inventors: Bhami Shenoy, South Grafton, MA (US); Danica Grujic, Boston, MA (US); Reena Patel, Woburn, MA (US); Vinney George, Arlington, MA (US); Margaret Mcgrath, Somerville, MA (US); Nazer Khalaf, Worcester, MA (US); Emma Watson, Somerville, MA (US)

(73) Assignee: Althea Technologies, Inc., San Diego, CA (US)

(21) Appl. No.: 12/934,392

(22) PCT Filed: Mar. 24, 2009
(86) PCT No.: PCT/US2009/038124

§ 371 Date: Jan. 31, 2011

Related U.S. Application Data
(60) Provisional application No. 61/039,018, filed on Mar. 24, 2008.

(51) Int. Cl. A61K 38/44; A61K 38/54; A61K 9/00; A61P 3/10; A61P 9/00; A61P 19/06; A61P 9/12; A61P 13/12; A61P 3/00

(52) U.S. Cl. 424/400; 424/94.4; 424/94.2

(57) ABSTRACT

Compositions containing uricase are disclosed. Methods of treating a disorder associated with elevated uric acid concentrations using uricase are also described. Compositions containing uricase and catalase are also disclosed. Methods of treating a disorder associated with elevated uric acid concentrations using uricase and catalase are also described.

### CROSS REFERENCE TO RELATED APPLICATIONS

This application claims priority to U.S. Application Ser. No. 61/039,018, filed on Mar. 24, 2008. The disclosure of the prior application is considered part of (and is incorporated by reference in) the disclosure of this application.

### BACKGROUND

Uric acid is the final product of purine metabolism in human beings. The condition of hyperuricemia is indicative of a high level of uric acid in the blood (>7 mg/dL). Humans can have higher levels of uric acid (hyperuricemia) because of a deficiency of the hepatic enzyme, uricase, and a lower fractional excretion of uric acid. Approximately two thirds of total body uric acid is produced endogenously, while the remaining one third is accounted for by dietary purines. Approximately 70% of the uric acid produced daily is excreted by the kidneys, while the rest is eliminated by the intestines.¹

### SUMMARY

The invention relates, in part, to compositions for reducing uric acid levels and methods of reducing uric acid concentrations, e.g., methods of treating disorders associated with elevated uric acid concentrations.

In one aspect, the disclosure features a composition that includes uricase and a pH increasing agent.

In one aspect, the disclosure features a composition that includes uricase and a pH increasing agent and further includes a hydrogen peroxide degrading enzyme (e.g., peroxidase or catalase). In some embodiments, the composition contains catalase (e.g., bovine catalase).

In some embodiments, the pH increasing agent contains bicarbonate or a salt thereof.

In some embodiments, the pH increasing agent contains sodium bicarbonate.

In some embodiments, the pH increasing agent contains carbonate or a salt thereof.

In some embodiments, the pH increasing agent contains an anti-acid (e.g., Aluminium hydroxide; Magnesium hydroxide; Aluminum hydroxide and magnesium hydroxide; Aluminum carbonate gel; Calcium carbonate; Sodium bicarbonate; Hydrotalcite; Bismuth subsalicylate; or Magaldrate+Simethicone).

In some embodiments, the pH increasing agent comprises a proton pump inhibitor (e.g., Omeprazole; Lansoprazole; Esomeprazole; Pantoprazole; or Rabeprazole).

In some embodiments, the composition is a pharmaceutical composition.

In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings.

In some embodiments, the uricase is crystalline.

In another aspect, the disclosure features a method of treating a disorder associated with elevated uric acid concentration in a subject. The method includes administering uricase and a pH increasing agent to a subject, wherein, e.g., prior to administering the uricase and the pH increasing agent to the subject, the uric acid concentration in the subject is elevated as compared to a standard.

In some embodiments, the pH increasing agent increases pH (e.g., stomach pH) to above about 5 (e.g., increases the pH to about 5, about 5.5, about 6, about 6.5, about 7, or about 7.5).

In some embodiments, the uricase and the pH increasing agent are administered at the same time. For example, the uricase and the pH increasing agent are administered at the same time in the same or separate dosage forms.

In some embodiments, the uricase is administered before (e.g., about 5, about 10, about 15, about 20, about 25, about 30, about 40, about 50, or about 60 minutes before) the pH increasing agent is administered.

In some embodiments, the pH increasing agent is administered before (e.g., about 5, about 10, about 15, about 20, about 25, about 30, about 40, about 50, about 60, about 90, or about 120 minutes before) the uricase is administered.

In some embodiments, the uric acid concentration is elevated in blood (e.g., prior to administration of the uricase and the pH increasing agent).

In some embodiments, the uric acid concentration is elevated in urine (e.g., prior to administration of the uricase and the pH increasing agent).

In some embodiments, the method includes lowering the uric acid concentration in the subject, wherein the lowering is compared to a standard (e.g., after administering the uricase and pH increasing agent, the uric acid concentration in the subject is lower than the uric acid concentration in the subject prior to administering the uricase and pH increasing agent).

In some embodiments, the pH increasing agent comprises bicarbonate or a salt thereof.

In some embodiments, the pH increasing agent contains sodium bicarbonate.

In some embodiments, the pH increasing agent contains carbonate or a salt thereof.

In some embodiments, the pH increasing agent contains an anti-acid (e.g., Aluminium hydroxide; Magnesium hydroxide; Aluminum hydroxide and magnesium hydroxide; Aluminum carbonate gel; Calcium carbonate; Sodium bicarbonate; Hydrotalcite; Bismuth subsalicylate; or Magaldrate+Simethicone).

In some embodiments, the pH increasing agent contains a proton pump inhibitor (e.g., Omeprazole; Lansoprazole; Esomeprazole; Pantoprazole; or Rabeprazole).

In some embodiments, the uricase and the pH increasing agent are administered in combination with a hydrogen peroxide degrading enzyme (e.g., peroxidase or catalase). In some embodiments, catalase (e.g., bovine catalase) is administered.

In some embodiments, the uricase and the pH increasing agent and the hydrogen peroxide degrading enzyme (e.g., peroxidase or catalase) are administered at the same time.

In some embodiments, the uricase and/or the pH increasing agent are administered before (e.g., about 5, about 10, about 15, about 20, about 25, about 30, about 40, about 50, about 60, about 90, or about 120 minutes before) the a hydrogen peroxide degrading enzyme (e.g., peroxidase or catalase) is administered.

In some embodiments, the uricase and/or the pH increasing agent are administered after (e.g., about 5, about 10, about 15, about 20, about 25, about 30, about 40, about 50, about 60, about 90, or about 120 minutes after) the hydrogen peroxide degrading enzyme (e.g., peroxidase or catalase) is administered.

In some embodiments, the uricase and the pH increasing agent are administered in combination with an additional agent.

In some embodiments, the additional agent contains a xanthine-oxidase inhibitor (e.g., allopurinol, 2-(3-cyano-4-isobutoxyphenyl)-4-methyl-5-thiazolecarboxylic acid (TEI-6720); febuxostat (a non-purine inhibitor; 2-[3-cyano-4-isobutoxyphenyl]-4-methylthiazole-5-carboxylic acid), oxypurinol, or pteridylaldehyde) or an uricosuric (e.g., probenecid, sulfinpyrazone, sulfinpyrazone, or fenofibrate).

In some embodiments, the additional agent contains a xanthine-oxidase inhibitor, wherein the xanthine-oxidase inhibitor contains allopurinol.

In some embodiments, the additional agent contains an uricosuric, wherein the uricosuric contains probenecid or sulfinpyrazone.

In some embodiments, the additional agent contains PEG-uricase.

In some embodiments, the additional agent contains ethylenediaminetetraacetic acid.

In some embodiments, the additional agent contains acetazolamide

In some embodiments, the additional agent contains a potassium supplement.

In some embodiments, the uricase and the pH increasing agent and the additional agent are administered at the same time.

In some embodiments, the uricase and/or the pH increasing agent are administered before (e.g., about 5, about 10, about 15, about 20, about 25, about 30, about 40, about 50, about 60, about 90, or about 120 minutes before) the additional agent is administered.

In some embodiments, the uricase and/or the pH increasing agent are administered after (e.g., about 5, about 10, about 15, about 20, about 25, about 30, about 40, about 50, about 60, about 90, or about 120 minutes after) the additional agent is administered.

In some embodiments, the disorder associated with elevated uric acid concentration includes (or is) hyperuricemia.

In some embodiments, the disorder associated with elevated uric acid concentration includes (or is) gout.

In some embodiments, the disorder associated with elevated uric acid concentration includes (or is) Lesch-Nyhan syndrome.

In some embodiments, the disorder associated with elevated uric acid concentration includes (or is) cardiovascular disease.

In some embodiments, the disorder associated with elevated uric acid concentration includes (or is) diabetes.

In some embodiments, the disorder associated with elevated uric acid concentration includes (or is) hypertension.

In some embodiments, the disorder associated with elevated uric acid concentration includes (or is) renal disease.

In some embodiments, the disorder associated with elevated uric acid concentration includes (or is) kidney stones.

In some embodiments, the disorder associated with elevated uric acid concentration includes (or is) hyperuricosuria.

In some embodiments, the disorder associated with elevated uric acid concentration includes (or is) uric acid nephrolithiasis.

In some embodiments, the disorder associated with elevated uric acid concentration includes (or is) metabolic syndrome.

In some embodiments, the disorder associated with elevated uric acid concentration includes (or is) tumor lysis syndrome.

In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

In one aspect, the disclosure features a composition that includes uricase, wherein the uricase has been stabilized (e.g., the uricase has increased stability in given conditions relative to a uricase that has not been stabilized), e.g., in an acidic environment, e.g., in acidic conditions, e.g., in the gastrointestina tract.

In some embodiments, the uricase is stabilized by use of a polyionic reagent.

In some embodiments, the uricase is stabilized by a polyionic coating.

In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane.

In some embodiments, the stabilized uricase comprises more than one polyionic coating, e.g., more than one coating with the same material and/or coatings with more than one material.

In some embodiments, the uricase comprises PMG and PAA coatings.

In some embodiments, the composition further includes a pH increasing agent.

In some embodiments, the pH increasing agent contains bicarbonate or a salt thereof.

In some embodiments, the pH increasing agent contains sodium bicarbonate.

In some embodiments, the pH increasing agent contains carbonate or a salt thereof.

In some embodiments, the pH increasing agent contains an anti-acid (e.g., Aluminium hydroxide; Magnesium hydroxide; Aluminum hydroxide and magnesium hydroxide; Aluminum carbonate gel; Calcium carbonate; Sodium bicarbonate; Hydrotalcite; Bismuth subsalicylate; or Magaldrate+Simethicone).

In some embodiments, the pH increasing agent comprises a proton pump inhibitor (e.g., Omeprazole; Lansoprazole; Esomeprazole; Pantoprazole; or Rabeprazole).

In some embodiments, the composition is a pharmaceutical composition.

In some embodiments, the uricase is crystalline.

In one aspect, the disclosure features a composition that includes a stabilized uricase and further includes a hydrogen peroxide degrading enzyme (e.g., peroxidase or catalase). In some embodiments, the composition contains catalase (e.g., bovine catalase).

In some embodiments, the uricase is stabilized by use of a polyionic reagent.

In some embodiments, the uricase is stabilized by a polyionic coating.

In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane.

In some embodiments, the stabilized uricase comprises more than one polyionic coating, e.g., more than one coating with the same material and/or coatings with more than one material.

In some embodiments, the uricase comprises PMG and PAA coatings.

In some embodiments, the uricase is crystalline.

In some embodiments, the composition further includes a pH increasing agent.

In some embodiments, the pH increasing agent contains bicarbonate or a salt thereof.

In some embodiments, the pH increasing agent contains sodium bicarbonate.

In some embodiments, the pH increasing agent contains carbonate or a salt thereof.

In some embodiments, the pH increasing agent contains an anti-acid (e.g., Aluminium hydroxide; Magnesium hydroxide; Aluminum hydroxide and magnesium hydroxide; Aluminum carbonate gel; Calcium carbonate; Sodium bicarbonate; Hydrotalcite; Bismuth subsalicylate; or Magaldrate+Simethicone).

In some embodiments, the pH increasing agent comprises a proton pump inhibitor (e.g., Omeprazole; Lansoprazole; Esomeprazole; Pantoprazole; or Rabeprazole).

In some embodiments, the composition is a pharmaceutical composition.

In some embodiments, the uricase is crystalline.

In some embodiments, the uricase has a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the uricase comprises more than one polyionic coating, e.g., more than one coating with the same material and/or coatings with more than one material. In some embodiments, the uricase comprises PMG and PAA coatings. In some embodiments, the uricase is crystalline.

In another aspect, the disclosure features a method of treating a disorder associated with elevated uric acid concentration in a subject. The method includes administering stabilized uricase to a subject, wherein, e.g., prior to administering the stabilized uricase to the subject, the uric acid concentration in the subject is elevated as compared to a standard.

In some embodiments, the uricase is stabilized by use of a polyionic reagent.

In some embodiments, the uricase is stabilized by a polyionic coating.

In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane.

In some embodiments, the stabilized uricase comprises more than one polyionic coating, e.g., more than one coating with the same material and/or coatings with more than one material.

In some embodiments, the uricase comprises PMG and PAA coatings.

In some embodiments, the method further includes administering a pH increasing agent.

In some embodiments, the pH increasing agent contains bicarbonate or a salt thereof.

In some embodiments, the pH increasing agent contains sodium bicarbonate.

In some embodiments, the pH increasing agent contains carbonate or a salt thereof.

In some embodiments, the pH increasing agent contains an anti-acid (e.g., Aluminium hydroxide; Magnesium hydroxide; Aluminum hydroxide and magnesium hydroxide; Aluminum carbonate gel; Calcium carbonate; Sodium bicarbonate; Hydrotalcite; Bismuth subsalicylate; or Magaldrate+Simethicone).

In some embodiments, the pH increasing agent comprises a proton pump inhibitor (e.g., Omeprazole; Lansoprazole; Esomeprazole; Pantoprazole; or Rabeprazole).

In some embodiments, the composition is a pharmaceutical composition.

In some embodiments, the uricase is crystalline.

In some embodiments, the pH increasing agent increases pH (e.g., stomach pH) to above about 5 (e.g., increases the pH to about 5, about 5.5, about 6, about 6.5, about 7, or about 7.5).

In some embodiments, the uricase and the pH increasing agent are administered at the same time. For example, the uricase and the pH increasing agent are administered at the same time in the same or separate dosage forms.

In some embodiments, the uricase is administered before (e.g., about 5, about 10, about 15, about 20, about 25, about 30, about 40, about 50, or about 60 minutes before) the pH increasing agent is administered.

In some embodiments, the pH increasing agent is administered before (e.g., about 5, about 10, about 15, about 20, about 25, about 30, about 40, about 50, about 60, about 90, or about 120 minutes before) the uricase is administered.

In some embodiments, the uric acid concentration is elevated in blood (e.g., prior to administration of the uricase and the pH increasing agent).

In some embodiments, the uric acid concentration is elevated in urine (e.g., prior to administration of the uricase and the pH increasing agent).

In some embodiments, the method includes lowering the uric acid concentration in the subject, wherein the lowering is compared to a standard (e.g., after administering the stabilized uricase, the uric acid concentration in the subject is lower than the uric acid concentration in the subject prior to administering the stabilized uricase).

In some embodiments, the pH increasing agent comprises bicarbonate or a salt thereof.

In some embodiments, the pH increasing agent contains sodium bicarbonate.

In some embodiments, the pH increasing agent contains carbonate or a salt thereof.

In some embodiments, the pH increasing agent contains an anti-acid (e.g., Aluminium hydroxide; Magnesium hydroxide; Aluminum hydroxide and magnesium hydroxide; Aluminum carbonate gel; Calcium carbonate; Sodium bicarbonate; Hydrotalcite; Bismuth subsalicylate; or Magaldrate+Simethicone).

In some embodiments, the pH increasing agent contains a proton pump inhibitor (e.g., Omeprazole; Lansoprazole; Esomeprazole; Pantoprazole; or Rabeprazole).

In some embodiments, the uricase and the pH increasing agent are administered in combination with a hydrogen peroxide degrading enzyme (e.g., peroxidase or catalase). In some embodiments, catalase (e.g., bovine catalase) is administered.

In some embodiments, the uricase and the pH increasing agent and the hydrogen peroxide degrading enzyme (e.g., peroxidase or catalase) are administered at the same time.

In some embodiments, the uricase and/or the pH increasing agent are administered before (e.g., about 5, about 10, about 15, about 20, about 25, about 30, about 40, about 50, about 60, about 90, or about 120 minutes before) the hydrogen peroxide degrading enzyme (e.g., peroxidase or catalase) is administered.

In some embodiments, the uricase and/or the pH increasing agent are administered after (e.g., about 5, about 10, about 15, about 20, about 25, about 30, about 40, about 50, about 60, about 90, or about 120 minutes after) the hydrogen peroxide degrading enzyme (e.g., peroxidase or catalase) is administered.

In some embodiments, the uricase and the pH increasing agent are administered in combination with an additional agent.

In some embodiments, the additional agent contains a xanthine-oxidase inhibitor (e.g., allopurinol, 2-(3-cyano-4-isobutoxyphenyl)-4-methyl-5-thiazolecarboxylic acid (TEI-6720); febuxostat (a non-purine inhibitor; 2-[3-cyano-4-isobutoxyphenyl]-4-methylthiazole-5-carboxylic acid), oxypurinol, or pteridylaldehyde) or an uricosuric (e.g., probenecid, sulfinpyrazone, sulfinpyrazone, or fenofibrate).

In some embodiments, the additional agent contains a xanthine-oxidase inhibitor, wherein the xanthine-oxidase inhibitor contains allopurinol.

In some embodiments, the additional agent contains an uricosuric, wherein the uricosuric contains probenecid or sulfinpyrazone.

In some embodiments, the additional agent contains PEG-uricase.

In some embodiments, the additional agent contains ethylenediaminetetraacetic acid.

In some embodiments, the additional agent contains acetazolamide

In some embodiments, the additional agent contains a potassium supplement.

In some embodiments, the uricase and the pH increasing agent and the additional agent are administered at the same time.

In some embodiments, the uricase and/or the pH increasing agent are administered before (e.g., about 5, about 10, about 15, about 20, about 25, about 30, about 40, about 50, about 60, about 90, or about 120 minutes before) the additional agent is administered.

In some embodiments, the uricase and/or the pH increasing agent are administered after (e.g., about 5, about 10, about 15, about 20, about 25, about 30, about 40, about 50, about 60, about 90, or about 120 minutes after) the additional agent is administered.

In some embodiments, the disorder associated with elevated uric acid concentration includes (or is) hyperuricemia.

In some embodiments, the disorder associated with elevated uric acid concentration includes (or is) gout.

In some embodiments, the disorder associated with elevated uric acid concentration includes (or is) Lesch-Nyhan syndrome.

In some embodiments, the disorder associated with elevated uric acid concentration includes (or is) cardiovascular disease.

In some embodiments, the disorder associated with elevated uric acid concentration includes (or is) diabetes.

In some embodiments, the disorder associated with elevated uric acid concentration includes (or is) hypertension.

In some embodiments, the disorder associated with elevated uric acid concentration includes (or is) renal disease.

In some embodiments, the disorder associated with elevated uric acid concentration includes (or is) kidney stones.

In some embodiments, the disorder associated with elevated uric acid concentration includes (or is) hyperuricosuria.

In some embodiments, the disorder associated with elevated uric acid concentration includes (or is) uric acid nephrolithiasis.

In some embodiments, the disorder associated with elevated uric acid concentration includes (or is) metabolic syndrome. In some embodiments, the disorder associated with elevated uric acid concentration includes (or is) tumor lysis syndrome.

In one aspect, the disclosure features a composition that includes uricase and a hydrogen peroxide degrading enzyme (e.g., peroxidase or catalase).

In some embodiments, the hydrogen peroxide degrading enzyme comprises catalase.

In some embodiments, the catalase comprises bovine catalase.

In some embodiments, the composition is a pharmaceutical composition.

In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

In another aspect, the disclosure features a method of treating a disorder associated with elevated uric acid concentration in a subject. The method includes administering uricase and hydrogen peroxide degrading enzyme (e.g., peroxidase or catalase) to a subject, wherein prior to administering the uricase and the hydrogen peroxide degrading enzyme (e.g., peroxidase or catalase) to the subject, the uric acid concentration in the subject is elevated as compared to a standard.

In some embodiments, the uricase and the hydrogen peroxide degrading enzyme (e.g., peroxidase or catalase) are administered at the same time. For example, the uricase and the hydrogen peroxide degrading enzyme (e.g., peroxidase or catalase) are administered at the same time in the same or separate dosage forms.

In some embodiments, the uricase is administered before (e.g., about 5, about 10, about 15, about 20, about 25, about 30, about 40, about 50, or about 60 minutes before) the hydrogen peroxide degrading enzyme (e.g., peroxidase or catalase) is administered.

In some embodiments, the hydrogen peroxide degrading enzyme (e.g., peroxidase or catalase) is administered before (e.g., about 5, about 10, about 15, about 20, about 25, about 30, about 40, about 50, about 60, about 90, or about 120 minutes before) the uricase is administered.

In some embodiments, the uric acid concentration is elevated in blood (e.g., prior to administration of the uricase and the hydrogen peroxide degrading enzyme (e.g., peroxidase or catalase)).

In some embodiments, the uric acid concentration is elevated in urine (e.g., prior to administration of the uricase and the hydrogen peroxide degrading enzyme (e.g., peroxidase or catalase)).

In some embodiments, the method includes lowering the uric acid concentration in the subject, wherein the lowering is compared to a standard (e.g., after administering the uricase and hydrogen peroxide degrading enzyme (e.g., peroxidase or catalase), the uric acid concentration in the subject is lower than the uric acid concentration in the subject prior to administering the uricase and hydrogen peroxide degrading enzyme (e.g., peroxidase or catalase)).

In some embodiments, the hydrogen peroxide degrading enzyme comprises catalase.

In some embodiments, the catalase comprises bovine catalase.

In some embodiments, uricase, a pH increasing agent, and a hydrogen peroxide degrading enzyme (e.g., peroxidase or catalase) are administered.

In some embodiments, the uricase and/or the hydrogen peroxide degrading enzyme (e.g., peroxidase or catalase) are administered before (e.g., about 5, about 10, about 15, about 20, about 25, about 30, about 40, about 50, about 60, about 90, or about 120 minutes before) the pH increasing agent is administered.

In some embodiments, the uricase and/or the hydrogen peroxide degrading enzyme (e.g., peroxidase or catalase) are administered after (e.g., about 5, about 10, about 15, about 20, about 25, about 30, about 40, about 50, about 60, about 90, about 120 minutes after) the pH increasing agent is administered.

In some embodiments, the pH increasing agent and/or the hydrogen peroxide degrading enzyme (e.g., peroxidase or catalase) are administered before (e.g., about 5, about 10, about 15, about 20, about 25, about 30, about 40, about 50, about 60, about 90, or about 120 minutes before) the uricase is administered.

In some embodiments, the pH increasing agent and/or the hydrogen peroxide degrading enzyme (e.g., peroxidase or catalase) are administered after (e.g., about 5, about 10, about 15, about 20, about 25, about 30, about 40, about 50, about 60, about 90, or about 120 minutes after) the uricase is administered.

In some embodiments, the uricase and the hydrogen peroxide degrading enzyme (e.g., peroxidase or catalase) are administered in combination with an additional agent.

In some embodiments, the additional agent contains a xanthine-oxidase inhibitor (e.g., allopurinol, 2-(3-cyano-4-isobutoxyphenyl)-4-methyl-5-thiazolecarboxylic acid (TEI-6720); febuxostat (a non-purine inhibitor; 2-[3-cyano-4-isobutoxyphenyl]-4-methylthiazole-5-carboxylic acid), oxypurinol, or pteridylaldehyde) or an uricosuric (e.g., probenecid, sulfinpyrazone, sulfinpyrazone, or fenofibrate).

In some embodiments, the additional agent contains a xanthine-oxidase inhibitor, wherein the xanthine-oxidase inhibitor contains allopurinol.

In some embodiments, the additional agent contains an uricosuric, wherein the uricosuric contains probenecid or sulfinpyrazone.

In some embodiments, the additional agent contains PEG-uricase.

In some embodiments, the additional agent contains ethylenediaminetetraacetic acid.

In some embodiments, the additional agent contains acetazolamide

In some embodiments, the additional agent contains a potassium supplement.

In some embodiments, the uricase and the hydrogen peroxide degrading enzyme (e.g., peroxidase or catalase) and the additional agent are administered at the same time.

In some embodiments, the uricase and/or the hydrogen peroxide degrading enzyme (e.g., peroxidase or catalase) are administered before (e.g., about 5, about 10, about 15, about 20, about 25, about 30, about 40, about 50, about 60, about 90, or about 120 minutes before) the additional agent is administered.

In some embodiments, the uricase and/or the hydrogen peroxide degrading enzyme (e.g., peroxidase or catalase) are administered after (e.g., about 5, about 10, about 15, about 20, about 25, about 30, about 40, about 50, about 60, about 90, or about 120 minutes after) the additional agent is administered.

In some embodiments, the disorder associated with elevated uric acid concentration includes (or is) hyperuricemia.

In some embodiments, the disorder associated with elevated uric acid concentration includes (or is) gout.

In some embodiments, the disorder associated with elevated uric acid concentration includes (or is) Lesch-Nyhan syndrome.

In some embodiments, the disorder associated with elevated uric acid concentration includes (or is) cardiovascular disease.

In some embodiments, the disorder associated with elevated uric acid concentration includes (or is) diabetes.

In some embodiments, the disorder associated with elevated uric acid concentration includes (or is) hypertension.

In some embodiments, the disorder associated with elevated uric acid concentration includes (or is) renal disease.

In some embodiments, the disorder associated with elevated uric acid concentration includes (or is) kidney stones.

In some embodiments, the disorder associated with elevated uric acid concentration includes (or is) hyperuricosuria.

In some embodiments, the disorder associated with elevated uric acid concentration includes (or is) uric acid nephrolithiasis.

In some embodiments, the disorder associated with elevated uric acid concentration includes (or is) metabolic syndrome.

In some embodiments, the disorder associated with elevated uric acid concentration includes (or is) tumor lysis syndrome. In some embodiments, the uricase is stabilized by use of a polyionic reagent.

In some embodiments, the uricase is stabilized by a polyionic coating.

In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane.

In some embodiments, the stabilized uricase comprises more than one polyionic coating, e.g., more than one coating with the same material and/or coatings with more than one material.

In some embodiments, the uricase comprises PMG and PAA coatings.

In some embodiments, the uricase is crystalline.

The composition contains uricase (urate oxidase) and a pH increasing agent. The composition can optionally include an additional agent, such as a xanthine-oxidase inhibitor, and/or an uricosuric. In some embodiments, the composition containing uricase, and a pH increasing agent can be administered with another agent(s) as part of a combination therapy; the other agent can be, e.g., a xanthine-oxidase inhibitor, and/or an uricosuric. The composition (or combination) can be used to treat or prevent uric acid-associated disorders, e.g., a metabolic disorder, e.g., metabolic syndrome, hyperuricemia (e.g., due to tumor lysis syndrome), gout (e.g., gouty arthritis), Lesch-Nyhan syndrome, cardiovascular disease, diabetes, hypertension, renal disease, metabolic syndrome, uric acid nephrolithiasis, or kidney stones. Uric acid-associated disorders, e.g., a metabolic disorder, e.g., metabolic syndrome, hyperuricemia (e.g., due to tumor lysis syndrome), gout (e.g., gouty arthritis), Lesch-Nyhan syndrome, tumor lysis syndrome, cardiovascular disease, diabetes, hypertension, renal disease, metabolic syndrome, uric acid nephrolithiasis, or kidney stones, can be treating by administering uricase and a pH increasing agent. In one embodiment, uricase and a pH increasing agent (alone or in combination with another agent) can be administered to a subject, e.g., a mammal, e.g., orally or directly to the stomach, to reduce uric acid levels. In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

In disclosure relates, in part, to a combination therapy, e.g., that can be used to treat a uric acid-associated disorder. The combination therapy can include a composition for reducing uric acid, wherein the composition contains uricase and a pH-increasing agent. The disclosure includes a method for reducing uric acid levels in a mammal by administering, e.g., orally administering, uricase and a pH-increasing agent. In some embodiments, the pH-increasing agent can be a carbonate, or a salt form thereof, or bicarbonate, or a salt form thereof.

Uricase and a pH increasing agent (alone or in combination with another agent) can be administered in a therapeutically effective amount. The uricase (alone or in combination with another agent) can lower the uric acid concentration in a subject. By the term “lower”, it is meant that the uric acid concentration is lowered relative to a standard. The standard can be, for example, the uric acid concentration present in the subject before the first administration of the uricase and pH increasing agent (alone or in combination with another agent). The uricase and pH increasing agent (alone or in combination with another agent) can lower the uric acid concentration by about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% as compared to the concentration that was present in the subject before administration of the uricase and pH increasing agent (alone or in combination with another agent). As another example, in a subject with elevated concentrations of uric acid (e.g., a subject with a uric acid concentration that is higher than what is considered normal by the American Medical Association, e.g., above 8.3 mg/dL (˜494 μmol/L)), the administration of the uricase and pH increasing agent (alone or in combination with another agent) can lower the uric acid concentration to a level that is considered to be normal by the American Medical Association (concentrations between 3.6 mg/dL (˜214 μmol/L) and 8.3 mg/dL (˜494 μmol/L) are considered normal).

As a further example, in a subject with elevated concentrations of uric acid (e.g., a subject with a uric acid concentration that classifies a subject as having hyperuricemia, e.g., blood uric acid levels above >7 mg/dL), the administration of the uricase and a pH increasing agent (alone or in combination with another agent) can lower the uric acid concentration to a level that is below 7 mg/dL.

As another example, the standard can be a cohort of subjects, e.g., subjects with gout, and the uricase can lower a subject's uric acid concentrations to a concentration that is below the average concentration for a cohort of subjects with gout.

In another aspect, the uricase and pH increasing agent (alone or in combination with another agent) can be administered in a therapeutically effective amount to a subject that exhibits a symptom of a disorder associated with elevated uric acid concentrations, e.g., a symptom of gout, e.g., tenderness or pain of a joint.

In one aspect, the invention provides a composition containing uricase and a pH increasing agent.

In one aspect, the invention provides a method of reducing uric acid concentration in a subject by administering uricase and a pH increasing agent. Administration of the uricase and pH increasing agent can cause a reduction of uric acid concentration by about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% as compared to a standard (examples of standards are provided above). In some embodiments, the composition is administered orally. In some embodiments, the uricase and/or pH increasing agent is administered as a suspension, dry powder, capsule, or tablet. In one embodiment, the method of reducing uric acid concentration in a mammal includes a step of assaying the uric acid concentration in a biological sample of the subject, such as a urine, blood, plasma, or serum sample. In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

In another aspect, the invention provides a method of treating, preventing, and/or slowing the progression of a disorder associated with elevated uric acid concentrations in a subject by administering uricase and pH increasing agent to the subject. In one embodiment, the disorder associated with elevated uric acid concentration is a metabolic disorder, e.g., hyperuricemia, gout (e.g., gouty arthritis), Lesch-Nyhan syndrome, cardiovascular disease, diabetes, hypertension, metabolic syndrome, uric acid nephrolithiasis, or kidney stones. In certain embodiments, the disorder is gout.

In another aspect, the invention provides a composition, e.g., a pharmaceutical composition, that includes uricase and a pH increasing agent.

In yet another aspect, the invention provides a method of treating a subject (e.g., a mammal, e.g., a human or non-human mammal) by administering an effective amount of a pharmaceutical composition that includes uricase and a pH increasing agent.

In some aspects, the disclosure provides the use of a composition described herein (e.g., a composition containing uricase and a pH increasing agent, alone or in combination with another agent described herein (e.g., a xanthine-oxidase inhibitor, and/or an uricosuric)) for use in treatment.

In some aspects, the disclosure provides the use of a composition described herein (e.g., a composition containing uricase and a pH increasing agent, alone or in combination with another agent described herein (e.g., a xanthine-oxidase inhibitor, and/or an uricosuric)) for the preparation of a medicament, e.g., for treating a condition described herein, e.g., a uric acid-associated disorder, e.g., a metabolic disorder, e.g., metabolic syndrome, hyperuricemia, gout (e.g., gouty arthritis), Lesch-Nyhan syndrome, tumor lysis syndrome, cardiovascular disease, diabetes, hypertension, hypertension, renal disease, metabolic syndrome, uric acid nephrolithiasis, or kidney stones.

In some aspects, the disclosure provides the use of uricase and a pH increasing agent, alone or in combination with another agent described herein (e.g., a xanthine-oxidase inhibitor, and/or an uricosuric), for use in treatment.

In some aspects, the disclosure provides the use of uricase and a pH increasing agent, alone or in combination with another agent described herein (e.g., a xanthine-oxidase inhibitor, and/or an uricosuric), for the preparation of a medicament, e.g., for treating a condition described herein, e.g., a uric acid-associated disorder, e.g., a metabolic disorder, e.g., metabolic syndrome, hyperuricemia, gout (e.g., gouty arthritis), Lesch-Nyhan syndrome, tumor lysis syndrome, cardiovascular disease, diabetes, hypertension, hypertension, renal disease, metabolic syndrome, uric acid nephrolithiasis, or kidney stones. In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

The composition contains uricase (urate oxidase) and a hydrogen peroxide degrading enzyme (e.g., peroxidase or catalase). The composition can optionally include an additional agent, such as a xanthine-oxidase inhibitor, and/or an uricosuric. In some embodiments, the composition containing uricase, and a hydrogen peroxide degrading enzyme (e.g., peroxidase or catalase) can be administered with another agent(s) as part of a combination therapy; the other agent can be, e.g., a xanthine-oxidase inhibitor, and/or an uricosuric. The composition (or combination) can be used to treat or prevent uric acid-associated disorders, e.g., a metabolic disorder, e.g., metabolic syndrome, hyperuricemia (e.g., due to tumor lysis syndrome), gout (e.g., gouty arthritis), Lesch-Nyhan syndrome, tumor lysis syndrome, cardiovascular disease, diabetes, hypertension, renal disease, metabolic syndrome, uric acid nephrolithiasis, or kidney stones. Uric acid-associated disorders, e.g., a metabolic disorder, e.g., metabolic syndrome, hyperuricemia (e.g., due to tumor lysis syndrome), gout (e.g., gouty arthritis), Lesch-Nyhan syndrome, tumor lysis syndrome, cardiovascular disease, diabetes, hypertension, renal disease, metabolic syndrome, uric acid nephrolithiasis, or kidney stones, can be treating by administering uricase and a hydrogen peroxide degrading enzyme (e.g., peroxidase or catalase). In one embodiment, uricase and a hydrogen peroxide degrading enzyme (e.g., peroxidase or catalase) (alone or in combination with another agent) can be administered to a subject, e.g., a mammal, e.g., orally or directly to the stomach, to reduce uric acid levels. In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

The composition contains stabilized uricase, wherein the uricase has been stabilized (e.g., the uricase has increased stability in given conditions relative to a uricase that has not been stabilized), e.g., in an acidic environment, e.g., in acidic conditions, e.g., in the gastrointestinal tract, as described herein. The composition can optionally include an additional agent, such as a xanthine-oxidase inhibitor, and/or an uricosuric. In some embodiments, the composition containing stabilized uricase, further contains a pH increasing agent. In some embodiments, the composition containing stabilized uricase, and a pH increasing agent can be administered with another agent(s) as part of a combination therapy; the other agent can be, e.g., a xanthine-oxidase inhibitor, and/or an uricosuric. The composition (or combination) can be used to treat or prevent uric acid-associated disorders, e.g., a metabolic disorder, e.g., metabolic syndrome, hyperuricemia (e.g., due to tumor lysis syndrome), gout (e.g., gouty arthritis), Lesch-Nyhan syndrome, tumor lysis syndrome, cardiovascular disease, diabetes, hypertension, renal disease, metabolic syndrome, uric acid nephrolithiasis, or kidney stones. Uric acid-associated disorders, e.g., a metabolic disorder, e.g., metabolic syndrome, hyperuricemia (e.g., due to tumor lysis syndrome), gout (e.g., gouty arthritis), Lesch-Nyhan syndrome, tumor lysis syndrome, cardiovascular disease, diabetes, hypertension, renal disease, metabolic syndrome, uric acid nephrolithiasis, or kidney stones, can be treating by administering uricase and a pH increasing agent. In one embodiment, uricase and a pH increasing agent (alone or in combination with another agent) can be administered to a subject, e.g., a mammal, e.g., orally or directly to the stomach, to reduce uric acid levels. In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

In disclosure relates, in part, to a combination therapy, e.g., that can be used to treat a uric acid-associated disorder. The combination therapy can include a composition for reducing uric acid, wherein the composition contains stabilized uricase. The composition can optionally include a pH-increasing agent. The disclosure includes a method for reducing uric acid levels in a mammal by administering, e.g., orally administering, stabilized uricase.

Stabilized uricase (alone or in combination with another agent) can be administered in a therapeutically effective amount. The uricase (alone or in combination with another agent) can lower the uric acid concentration in a subject. By the term “lower”, it is meant that the uric acid concentration is lowered relative to a standard. The standard can be, for example, the uric acid concentration present in the subject before the first administration of the stabilized uricase (alone or in combination with another agent). The stabilized uricase (alone or in combination with another agent) can lower the uric acid concentration by about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% as compared to the concentration that was present in the subject before administration of the stabilized uricase (alone or in combination with another agent). As another example, in a subject with elevated concentrations of uric acid (e.g., a subject with a uric acid concentration that is higher than what is considered normal by the American Medical Association, e.g., above 8.3 mg/dL (˜494 μmol/L)), the administration of the stabilized uricase (alone or in combination with another agent) can lower the uric acid concentration to a level that is considered to be normal by the American Medical Association (concentrations between 3.6 mg/dL (˜214 μmol/L) and 8.3 mg/dL (˜494 μmol/L) are considered normal).

As a further example, in a subject with elevated concentrations of uric acid (e.g., a subject with a uric acid concentration that classifies a subject as having hyperuricemia, e.g., blood uric acid levels above >7 mg/dL), the administration of the stabilized uricase (alone or in combination with another agent) can lower the uric acid concentration to a level that is below 7 mg/dL.

As another example, the standard can be a cohort of subjects, e.g., subjects with gout, and the uricase can lower a subject's uric acid concentrations to a concentration that is below the average concentration for a cohort of subjects with gout.

In another aspect, the stabilized uricase (alone or in combination with another agent) can be administered in a therapeutically effective amount to a subject that exhibits a symptom of a disorder associated with elevated uric acid concentrations, e.g., a symptom of gout, e.g., tenderness or pain of a joint. In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

In one aspect, the invention provides a composition containing stabilized uricase. In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

In one aspect, the invention provides a method of reducing uric acid concentration in a subject by administering stabilized uricase. Administration of the stabilized uricase can cause a reduction of uric acid concentration by about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% as compared to a standard (examples of standards are provided above). In some embodiments, the composition is administered orally. In some embodiments, the stabilized uricase is administered as a suspension, dry powder, capsule, or tablet. In one embodiment, the method of reducing uric acid concentration in a mammal includes a step of assaying the uric acid concentration in a biological sample of the subject, such as a urine, blood, plasma, or serum sample. In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

In another aspect, the invention provides a method of treating, preventing, and/or slowing the progression of a disorder associated with elevated uric acid concentrations in a subject by administering stabilized uricase. In one embodiment, the disorder associated with elevated uric acid concentration is a metabolic disorder, e.g., hyperuricemia, gout (e.g., gouty arthritis), Lesch-Nyhan syndrome, tumor lysis syndrome, cardiovascular disease, diabetes, hypertension, metabolic syndrome, uric acid nephrolithiasis, or kidney stones. In certain embodiments, the disorder is gout. In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

In another aspect, the invention provides a composition, e.g., a pharmaceutical composition, that includes stabilized uricase. In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

In yet another aspect, the invention provides a method of treating a subject (e.g., a mammal, e.g., a human or non-human mammal) by administering an effective amount of a pharmaceutical composition that includes stabilized uricase. In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

In some aspects, the disclosure provides the use of a composition described herein (e.g., a composition containing stabilized uricase, alone or in combination with another agent described herein (e.g., a xanthine-oxidase inhibitor, and/or an uricosuric)) for use in treatment. In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

In some aspects, the disclosure provides the use of a composition described herein (e.g., a composition containing stabilized uricase, alone or in combination with another agent described herein (e.g., a xanthine-oxidase inhibitor, and/or an uricosuric)) for the preparation of a medicament, e.g., for treating a condition described herein, e.g., a uric acid-associated disorder, e.g., a metabolic disorder, e.g., metabolic syndrome, hyperuricemia, gout (e.g., gouty arthritis), Lesch-Nyhan syndrome, tumor lysis syndrome, cardiovascular disease, diabetes, hypertension, hypertension, renal disease, metabolic syndrome, uric acid nephrolithiasis, or kidney stones. In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

In some aspects, the disclosure provides the use of stabilized uricase and, alone or in combination with another agent described herein (e.g., a xanthine-oxidase inhibitor, and/or an uricosuric), for use in treatment. In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

In some aspects, the disclosure provides the use of stabilized uricase, alone or in combination with another agent described herein (e.g., a xanthine-oxidase inhibitor, and/or an uricosuric), for the preparation of a medicament, e.g., for treating a condition described herein, e.g., a uric acid-associated disorder, e.g., a metabolic disorder, e.g., metabolic syndrome, hyperuricemia, gout (e.g., gouty arthritis), Lesch-Nyhan syndrome, tumor lysis syndrome, cardiovascular disease, diabetes, hypertension, hypertension, renal disease, metabolic syndrome, uric acid nephrolithiasis, or kidney stones. In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

The composition contains uricase (urate oxidase) and a hydrogen peroxide degrading enzyme (e.g., peroxidase or catalase). The composition can optionally include an additional agent, such as a xanthine-oxidase inhibitor, and/or an uricosuric. In some embodiments, the composition contains catalase (e.g., bovine catalase). In some embodiments, the composition containing uricase, and a hydrogen peroxide degrading enzyme (e.g., peroxidase or catalase) can be administered with another agent(s) as part of a combination therapy; the other agent can be, e.g., a xanthine-oxidase inhibitor, and/or an uricosuric. The composition (or combination) can be used to treat or prevent uric acid-associated disorders, e.g., a metabolic disorder, e.g., metabolic syndrome, hyperuricemia (e.g., due to tumor lysis syndrome), gout (e.g., gouty arthritis), Lesch-Nyhan syndrome, tumor lysis syndrome, cardiovascular disease, diabetes, hypertension, renal disease, metabolic syndrome, uric acid nephrolithiasis, or kidney stones. Uric acid-associated disorders, e.g., a metabolic disorder, e.g., metabolic syndrome, hyperuricemia (e.g., due to tumor lysis syndrome), gout (e.g., gouty arthritis), Lesch-Nyhan syndrome, cardiovascular disease, diabetes, hypertension, renal disease, metabolic syndrome, uric acid nephrolithiasis, or kidney stones, can be treating by administering uricase and a hydrogen peroxide degrading enzyme (e.g., peroxidase or catalase). In one embodiment, uricase and a hydrogen peroxide degrading enzyme (e.g., peroxidase or catalase) (alone or in combination with another agent) can be administered to a subject, e.g., a mammal, e.g., orally or directly to the stomach, to reduce uric acid levels. In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

In disclosure relates, in part, to a combination therapy, e.g., that can be used to treat a uric acid-associated disorder. The combination therapy can include a composition for reducing uric acid, wherein the composition contains uricase and a hydrogen peroxide degrading enzyme (e.g., peroxidase or catalase). The disclosure includes a method for reducing uric acid levels in a mammal by administering, e.g., orally administering, uricase and a hydrogen peroxide degrading enzyme (e.g., peroxidase or catalase).

Uricase and a hydrogen peroxide degrading enzyme (e.g., peroxidase or catalase) (alone or in combination with another agent) can be administered in a therapeutically effective amount. The uricase and hydrogen peroxide degrading enzyme (e.g., peroxidase or catalase) (alone or in combination with another agent) can lower the uric acid concentration in a subject. By the term “lower”, it is meant that the uric acid concentration is lowered relative to a standard. The standard can be, for example, the uric acid concentration present in the subject before the first administration of the uricase and hydrogen peroxide degrading enzyme (e.g., peroxidase or catalase) (alone or in combination with another agent). The uricase and hydrogen peroxide degrading enzyme (e.g., peroxidase or catalase) (alone or in combination with another agent) can lower the uric acid concentration by about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% as compared to the concentration that was present in the subject before administration of the uricase and hydrogen peroxide degrading enzyme (e.g., peroxidase or catalase) (alone or in combination with another agent). As another example, in a subject with elevated concentrations of uric acid (e.g., a subject with a uric acid concentration that is higher than what is considered normal by the American Medical Association, e.g., above 8.3 mg/dL (˜494 μmol/L)), the administration of the uricase and hydrogen peroxide degrading enzyme (e.g., peroxidase or catalase) (alone or in combination with another agent) can lower the uric acid concentration to a level that is considered to be normal by the American Medical Association (concentrations between 3.6 mg/dL (˜214 μmol/L) and 8.3 mg/dL (˜494 μmol/L) are considered normal).

As a further example, in a subject with elevated concentrations of uric acid (e.g., a subject with a uric acid concentration that classifies a subject as having hyperuricemia, e.g., blood uric acid levels above >7 mg/dL), the administration of the uricase and a hydrogen peroxide degrading enzyme (e.g., peroxidase or catalase) (alone or in combination with another agent) can lower the uric acid concentration to a level that is below 7 mg/dL.

As another example, the standard can be a cohort of subjects, e.g., subjects with gout, and the uricase can lower a subject's uric acid concentrations to a concentration that is below the average concentration for a cohort of subjects with gout.

In another aspect, the uricase and hydrogen peroxide degrading enzyme (e.g., peroxidase or catalase) (alone or in combination with another agent) can be administered in a therapeutically effective amount to a subject that exhibits a symptom of a disorder associated with elevated uric acid concentrations, e.g., a symptom of gout, e.g., tenderness or pain of a joint. In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

In one aspect, the invention provides a composition containing uricase and a hydrogen peroxide degrading enzyme (e.g., peroxidase or catalase). In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

In one aspect, the invention provides a method of reducing uric acid concentration in a subject by administering uricase and a hydrogen peroxide degrading enzyme (e.g., peroxidase or catalase). Administration of the uricase and hydrogen peroxide degrading enzyme (e.g., peroxidase or catalase) can cause a reduction of uric acid concentration by about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% as compared to a standard (examples of standards are provided above). In some embodiments, the composition is administered orally. In some embodiments, the uricase and/or hydrogen peroxide degrading enzyme (e.g., peroxidase or catalase) is administered as a suspension, dry powder, capsule, or tablet. In one embodiment, the method of reducing uric acid concentration in a mammal includes a step of assaying the uric acid concentration in a biological sample of the subject, such as a urine, blood, plasma, or serum sample. In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

In another aspect, the invention provides a method of treating, preventing, and/or slowing the progression of a disorder associated with elevated uric acid concentrations in a subject by administering uricase and hydrogen peroxide degrading enzyme (e.g., peroxidase or catalase) to the subject. In one embodiment, the disorder associated with elevated uric acid concentration is a metabolic disorder, e.g., hyperuricemia, gout (e.g., gouty arthritis), Lesch-Nyhan syndrome, tumor lysis syndrome, cardiovascular disease, diabetes, hypertension, metabolic syndrome, uric acid nephrolithiasis, or kidney stones. In certain embodiments, the disorder is gout. In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

In another aspect, the invention provides a composition, e.g., a pharmaceutical composition that includes uricase and a hydrogen peroxide degrading enzyme (e.g., peroxidase or catalase). In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

In yet another aspect, the invention provides a method of treating a subject (e.g., a mammal, e.g., a human or non-human mammal) by administering an effective amount of a pharmaceutical composition that includes uricase and a hydrogen peroxide degrading enzyme (e.g., peroxidase or catalase). In some embodiments, the uricase has a polyionic coating. In some embodiments, the uricase is stabilized by use of a polyionic reagent. In some embodiments, the uricase is stabilized by a polyionic coating. In some embodiments, the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), or PVS: Polyvinylsiloxane. In some embodiments, the stabilized uricase comprises more than one polyionic coating, e.g., more than one coating with the same material and/or coatings with more than one material. In some embodiments, the uricase comprises PMG and PAA coatings. In some embodiments, the uricase is crystalline.

In some aspects, the disclosure provides the use of a composition described herein (e.g., a composition containing uricase and a hydrogen peroxide degrading enzyme (e.g., peroxidase or catalase), alone or in combination with another agent described herein (e.g., a xanthine-oxidase inhibitor, and/or an uricosuric)) for use in treatment.

In some aspects, the disclosure provides the use of a composition described herein (e.g., a composition containing uricase and a hydrogen peroxide degrading enzyme (e.g., peroxidase or catalase), alone or in combination with another agent described herein (e.g., a xanthine-oxidase inhibitor, and/or an uricosuric)) for the preparation of a medicament, e.g., for treating a condition described herein, e.g., a uric acid-associated disorder, e.g., a metabolic disorder, e.g., metabolic syndrome, hyperuricemia, gout (e.g., gouty arthritis), Lesch-Nyhan syndrome, cardiovascular disease, diabetes, hypertension, hypertension, renal disease, metabolic syndrome, uric acid nephrolithiasis, or kidney stones.

In some aspects, the disclosure provides the use of uricase and a hydrogen peroxide degrading enzyme (e.g., peroxidase or catalase), alone or in combination with another agent described herein (e.g., a xanthine-oxidase inhibitor, and/or an uricosuric), for use in treatment.

In some aspects, the disclosure provides the use of uricase and a hydrogen peroxide degrading enzyme (e.g., peroxidase or catalase), alone or in combination with another agent described herein (e.g., a xanthine-oxidase inhibitor, and/or an uricosuric), for the preparation of a medicament, e.g., for treating a condition described herein, e.g., a uric acid-associated disorder, e.g., a metabolic disorder, e.g., metabolic syndrome, hyperuricemia, gout (e.g., gouty arthritis), Lesch-Nyhan syndrome, cardiovascular disease, diabetes, hypertension, hypertension, renal disease, metabolic syndrome, uric acid nephrolithiasis, or kidney stones.

All publications, patent applications, patents, and other references mentioned herein are incorporated by reference in their entirety. In the case of conflict, the present specification, including definitions, controls. In addition, the materials, methods, and examples are illustrative only and not intended to be limiting.

The details of one or more embodiments of the invention are set forth in the accompanying drawings and the description below.

### DESCRIPTION OF THE DRAWINGS

FIG. 1. Reduction of hyperuricemia in Uox^{-/-} mice with daily oral therapy with uricase combined with pH modifying agent (mixture 1).

FIG. 2. Reduction in hyperuricosuria in Uox^{-/-} mice with daily oral therapy with uricase combined with pH modifying agent (mixture 1).

FIG. 3. Reduction in hyperuricemia in Uox^{-/-} mice with daily oral therapy with uricase combined with pH modifying agent (mixture 2).

FIG. 4. Reduction in hyperuricosuria in Uox^{-/-} mice with daily oral therapy with uricase combined with pH modifying agent (mixture 2).

FIG. 5. Comparison study: Efficacy of daily oral therapy of uricase with and without pH modifying agent in Uox^{-/-} mice.

FIG. 6 is a line graph showing uricase activity comparisons at different pH values.

FIGS. 7(A) and 7(B) are graphs showing pH stability comparisons for the uricases tested.

FIGS. 8(A) and 8(B) are graphs showing stability against protease trypsin.

FIGS. 9(A) and 9(B) are graphs showing stability against protease chymotrypsin.

FIG. 10 is a graph showing pH stability of coated uricase crystals.

FIG. 11 is a graph showing Uricase activity after incubation with chymotrypsin.

FIG. 12 is a graph showing plasma uric acid in Uox−/− mice treated with formulated uricase. Uric acid in Uox−/− control mice and mice treated for 19 days with soluble uricase protected with 1% bicarbonate salt in the food and water. Each bar represents the mean value±SE. Significant difference in uric acid levels is shown between the control group (n=8) and the uricase treatment group (n=8), P<0.05.

FIG. 13 is a graph showing Uric acid excretion in Uox−/− mice treated with formulated uricase. Urate excretion in control mice and mice treated for 19 days with soluble uricase protected with 1% bicarbonate given in the food and water. Each bar represents the mean value±SE. Significant difference in urine uric acid levels shown between control group (n=8) and uricase treated mice (n=8) P<0.05.

FIG. 14 is a graph showing Uric acid excretion in Uox−/− mice treated with formulated uricase. Urate excretion in control mice and mice treated for 21 days with soluble uricase protected with 1% bicarbonate given with food and water. Each bar represents mean value±SE. *Significant difference in urine uric acid levels shown between control group and uricase treated mice. (P<0.05) Each group had n=8 mice.

FIG. 15 is a graph showing plasma Uric acid in Uox−/− mice treated with formulated uricase. Uric acid in Uox−/− control mice and mice treated for 22 days with soluble uricase protected with 1% bicarbonate salt in the food and water. Each bar represents the mean value±SE, n=8 mice per group.

FIG. 16 is a graph showing Uric acid excretion in Uox−/− mice treated with soluble uricase. Urate excretion in Uox−/− control mice and mice treated with uricase mixed in the food (100 mg/3.5 g food with/without 1% bicarbonate) for 18 days. Each bar represents the mean value±SE. *Significant difference in urine uric acid levels shown between control group and uricase treated mice. (P<0.05) Control group (n=8), 100 mg uricase (n=8) 100 mg uricase +1% bicarbonate (n=8).

FIG. 17 is a graph showing plasma Uric acid in Uox−/− mice treated with soluble uricase. Uric acid in Uox−/− and control mice treated for 19 days with soluble uricase with or without 1% bicarbonate given in the food and water. Each bar represents the mean value±SE. Control group (n=8); 100 mg uricase (n=9), 100 mg uricase & 1% bicarbonate (n=12) mice.

FIG. 18 is a graph showing Plasma Uric acid in Uox−/− mice treated either with allopurinol or formulated uricase. Plasma Uric acid in Uox−/− control mice and mice treated for 10 days with allopurinol and soluble uricase protected with 1% bicarbonate given with food. Normalization of hyperuricemia in mice fed with 200 mg uricase A (UrA, 2000 U /mouse) compared to controls(CONT) (P<0.05). Normal levels of plasma uric acid are <2 mg/dL. Each bar represents mean value±SE. Significant difference in uric acid levels shown between control group and uricase and allopurinol treated mice (P<0.05). Control group n=(8); soluble uricase n=(8), allopurinoln=(8) and ContCont n=(6).

FIG. 19 is a graph showing Uric acid excretion in Uox−/− mice treated either allopurinol or formulated uricase. Excretion of urtae in Uox−/− control mice and mice treated for 10 days with allopurinol and soluble uricase protected with 1% bicarbonate given with food. Normalization of hyperuricosuria with uricase (P<0.05). Normal levels of urine uric acid are ˜2-3 mg/24 h. Each bar represents mean value±SE. Significant difference in uric acid levels shown between control group, uricase and allopurinol treated mice (P<0.05). Control group n=(8); soluble uricase n=(8), allopurinol n=(8) and ContCont n=(6).

FIG. 20 is a graph showing Uric acid excretion in Uox−/− mice treated orally with crystalline coated uricase Uric acid excretion in Uox−/− control mice and mice treated for 6 days orally with crystalline coated uricase mixed with 1% bicarbonate given with the food Normal level of urine uric acid is −2-3 mg/24 h. Each bar represents the mean value±SE. A significant difference in uric acid levels shown between control group, uricase and allopurinol treated mice. (P<0.05) Each group had n=(6) mice.

FIG. 21 is a graph showing Plasma uric acid in Uox−/− mice treated orally with crystalline coated uricase. Uric acid in Uox−/− control mice and mice treated for 6 days orally with crystalline coated uricase mixed with 1% bicarbonate given with the food. Each bar represents mean value±SE. n=(6) mice per group.

### DETAILED DESCRIPTION

The present invention is based, in part, on the discovery that administering uricase and a pH-increasing agent (optionally in combination with an additional treatment) can reduce a uric acid-associated disorder, or a symptom thereof, in a subject. Methods of administering uricase and a pH increasing agent to treat various uric acid-related disorders are described herein. Additionally, compositions containing uricase and a pH increasing agent (optionally in combination with an additional treatment), and uses thereof, are provided. Compositions containing uricase and a hydrogen peroxide degrading enzyme (e.g., peroxidase or catalase), and methods of using uricase and a hydrogen peroxide degrading enzyme (e.g., peroxidase or catalase), are also described.

As used herein, a “biological sample” is biological material collected from cells, tissues, organs, or organisms, for example, to detect an analyte. Exemplary biological samples include a fluid, cell, or tissue sample. Biological fluids include, for example, serum, blood, plasma, saliva, urine, or sweat. Cell or tissue samples include biopsy, tissue, cell suspension, or other specimens and samples, such as clinical samples.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. High stringency conditions (3) are the preferred conditions and the ones that should be used unless otherwise specified.

The term “subject” refers to any mammal, including but not limited to, any animal classified as such, including humans, non human primates, primates, baboons, chimpanzees, monkeys, rodents (e.g., mice, rats), rabbits, cats, dogs, horses, cows, sheep, goats, pigs, etc.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80% 90% (w/w) pure, even more preferably, 90 to 95% pure; and, most preferably, at least 95%, 96%, 97%, 98%, 99%, 99.5%, 99.8% or 100% (w/w) pure.

As used herein, the term “about” refers to up to ±10% of the value qualified by this term. For example, about 50 mM refers to 50 mM±5 mM; about 4% refers to 4%±0.4%.

As used herein, “uric acid-associated disorder” refers to a disease or disorder typically associated with elevated levels of uric acid, including, but not limited to a metabolic disorder, e.g., metabolic syndrome, hyperuricemia, gout (e.g., gouty arthritis), Lesch-Nyhan syndrome, cardiovascular disease, diabetes, hypertension, renal disease, metabolic syndrome, uric acid nephrolithiasis, or kidney stones. Such disorders may optionally be acute or chronic. Elevated levels refer to levels that are higher than levels that are considered normal by the American Medical Association, although significantly lower levels are common in vegetarians due to a decreased intake of purine-rich meat.

“Uric acid” is also known as urate (the two terms are used interchangeably herein). Humans produce large quantities of uric acid. In human blood, uric acid concentrations between 3.6 mg/dL (˜214 μmol/L) and 8.3 mg/dL (˜494 μmol/L) (1 mg/dL=59.48 μmol/L) are considered normal by the American Medical Association. Uric acid concentrations can be measured in samples from a subject, e.g., blood or urine samples, using known methods.

The terms “therapeutically effective dose,” and “therapeutically effective amount,” refer to that amount of a compound that results in prevention, delay of onset of symptoms, or amelioration of symptoms of an oxalate-related condition, including hyperoxaluria, such as primary hyperoxaluria or enteric hyperoxaluria. A therapeutically effective amount will, for example, be sufficient to treat, prevent, reduce the severity, delay the onset, and/or reduce the risk of occurrence of one or more symptoms of a disorder associated with elevated oxalate concentrations. The effective amount can be determined by methods well known in the art and as described in subsequent sections of this description.

The terms “treatment” and “therapeutic method” refer to treatment of an existing disorder and/or prophylactic/preventative measures. Those in need of treatment may include individuals already having a particular medical disorder, as well as those at risk or having, or who may ultimately acquire the disorder. The need for treatment is assessed, for example, by the presence of one or more risk factors associated with the development of a disorder, the presence or progression of a disorder, or likely receptiveness to treatment of a subject having the disorder. Treatment may include slowing or reversing the progression of a disorder.

The term “treating” refers to administering a therapy in an amount, manner, and/or mode effective to improve or prevent a condition, symptom, or parameter associated with a disorder (e.g., a disorder described herein) or to prevent onset, progression, or exacerbation of the disorder, to either a statistically significant degree or to a degree detectable to one skilled in the art. Accordingly, treating can achieve therapeutic and/or prophylactic benefits. An effective amount, manner, or mode can vary depending on the subject and may be tailored to the subject.

A subject who is at risk for, diagnosed with, or who has a uric acid-associated disorder, e.g., a disorder disclosed herein, be administered a therapy that includes a uricase and a pH increasing agent in an amount and for a time to provide an overall therapeutic effect. The uricase and pH increasing agent can be administered alone or in combination with another agent(s). In the case of a combination therapy, the amounts and times of administration can be those that provide, e.g., a synergistic therapeutic effect, or an additive therapeutic effect. Further, the administration of the uricase and the pH increasing agent (with or without the additional agent) can be used as a primary, e.g., first line treatment, or as a secondary treatment, e.g., for subjects who have an inadequate response to a previously administered therapy (i.e., a therapy other than one with a uricase). In some embodiments, a uricase and a pH increasing agent can be used in combination with a xanthine-oxidase inhibitor and/or an uricosuric and/or an antacid and/or a proton pump inhibitor.

A subject who is at risk for, diagnosed with, or who has a uric acid-associated disorder, e.g., a disorder disclosed herein, be administered a therapy that includes a uricase and a hydrogen peroxide degrading enzyme (e.g., peroxidase or catalase) in an amount and for a time to provide an overall therapeutic effect. The uricase and hydrogen peroxide degrading enzyme (e.g., peroxidase or catalase) can be administered alone or in combination with another agent(s). In the case of a combination therapy, the amounts and times of administration can be those that provide, e.g., a synergistic therapeutic effect, or an additive therapeutic effect. Further, the administration of the uricase and the hydrogen peroxide degrading enzyme (e.g., peroxidase or catalase) (with or without the additional agent) can be used as a primary, e.g., first line treatment, or as a secondary treatment, e.g., for subjects who have an inadequate response to a previously administered therapy (i.e., a therapy other than one with a uricase). In some embodiments, a uricase and a hydrogen peroxide degrading enzyme (e.g., peroxidase or catalase) can be used in combination with a xanthine-oxidase inhibitor and/or an uricosuric and/or an antacid and/or a proton pump inhibitor.

Uric Acid (Urate)

Uric acid is an end product of purine metabolism. Xanthine oxidase oxidizes oxypurines such as xanthine and hypoxanthine to uric acid. In humans and higher primates, uric acid is the final oxidation product of purine catabolism. In most other mammals, uricase (uricase) further oxidizes uric acid to allantoin. Contrary to other mammals, humans have lost the capacity to metabolize urate by hepatic uricase, due to mutational silencing of the enzyme. In addition, a peculiar renal handling, featured by relevant tubular reabsorption, sets plasma levels at these high levels. A fascinating hypothesis, supported by some experimental works, justifies this high concentration with urate acting as an effective scavenger against reactive oxygen species, namely hydroxyl-radicals and peroxynitrite in micro-vascular endothelium and in encephalic structures. However, in both plasma and urine, uric acid may exhibit increases as high as to reach saturation, with an ensuing risk of gout in plasma, and urolithiasis in urine.

The loss of uricase in higher primates parallels the similar loss of the ability to synthesize ascorbic acid. This may be because in higher primates uric acid partially replaces ascorbic acid. Both uric acid and ascorbate are strong reducing agents and potent antioxidants. In humans, about half the antioxidant capacity of plasma comes from urate.

Urate body pool is about 1-1.2 g, daily turnover being 0.6-0.7 g. Two-thirds of the newly produced uric acid is excreted in urine, while the remaining one third has a biliary or intestinal elimination or undergoes bacterial uricolysis. It emerges, therefore, that the kidney is the main regulator of uric acid balance.

Humans produce large quantities of uric acid. In human blood, uric acid concentrations between 3.6 mg/dL (˜214 μmol/L) and 8.3 mg/dL (˜494 μmol/L) (1 mg/dL=59.48 μmol/L) are considered normal by the American Medical Association, although significantly lower levels are common in vegetarians due to a decreased intake of purine-rich meat.

Uric acid is a weak organic acid of molecular weight 168 Daltons, with dissociation constants pK^{a}1=5.75 and pKa2=10.3 [1]. Therefore, at physiological blood pH, almost all the urate species are in the form of monovalent-anion. The solubility of urate in blood is about 7.0 mg/dL, above which it may deposit in tissues as monosodium-urate-monohydrate. Only about 4-5% of urate is bound to plasma proteins. Relative to other mammals, humans have high urate levels in plasma, ranging between 3.5 and 7.5 mg/dL (200-450 μmol/L), males having 1.2 times greater urate levels than healthy females.

Disorders associated with high uric acid levels include metabolic syndrome, hyperuricemia, gout (e.g., gouty arthritis), Lesch-Nyhan syndrome, cardiovascular disease, diabetes, hypertension, renal disease, metabolic syndrome, uric acid nephrolithiasis, or kidney stones. Such disorders can be treated with a uricase described herein and a pH increasing agent, e.g., a composition (e.g., a pharmaceutical composition) containing uricase and a pH increasing agent. In addition, such disorders can be treated by a combination of uricase, a pH increasing a gent, and another agent (e.g., a xanthine-oxidase inhibitor and/or an uricosuric and/or an antacid and/or a proton pump inhibitor).

Disorders associated with high uric acid levels include metabolic syndrome, hyperuricemia, gout (e.g., gouty arthritis), Lesch-Nyhan syndrome, cardiovascular disease, diabetes, hypertension, renal disease, metabolic syndrome, uric acid nephrolithiasis, or kidney stones. Such disorders can be treated with a uricase described herein and a hydrogen peroxide degrading enzyme (e.g., peroxidase or catalase), e.g., a composition (e.g., a pharmaceutical composition) containing uricase and a hydrogen peroxide degrading enzyme (e.g., peroxidase or catalase). In addition, such disorders can be treated by a combination of uricasem, a hydrogen peroxide degrading enzyme (e.g., peroxidase or catalase), and another agent (e.g., a xanthine-oxidase inhibitor and/or an uricosuric and/or an antacid and/or a proton pump inhibitor).

Uric Acid Elimination in the Urine

Uric acid, a weak organic acid, has very low pH-dependent solubility in aqueous solutions. About 70% of urate elimination occurs in urine, the kidney standing as a major determinant of plasma levels. The complex renal handling results in a fractional clearance of less than 10%. Recently identified urate-specific transporter/channels are involved in tubular handling and extracellular transport. Extracellular fluid, rather than urine output, is the main regulator of urate excretion. A number of interfering agents, including widely used drugs such as aspirin, losartan, diuretics, may decrease or increase urate elimination.⁴ Hyperuricemia induced by hypouricosuria often accompanies the metabolic syndrome, and insulin resistance has been hypothesized as the common underlying defect. Hyperuricosuria, associated with dehydration or exercise, results in acute uric acid nephropathy, and causes an obstructive acute renal failure (ARF). This reversible ARF can be prevented by forced hydration with bicarbonate or saline solutions. Renal hypouricemia, due to mutations of urate transporter, is a rare cause of exercise-induced ARF. The existence of chronic urate nephropathy, gouty nephropathy, is still under discussion. Uric acid nephrolithiasis results from super saturation, strongly influenced by low urine pH, rather than altered urate turnover. Alkali and fluid intake prove successful in managing uric acid stones.

Renal Handling of Uric Acid

Most of the uric acid generated daily is excreted by the kidney, which accounts for about 70% of urate elimination, with some 30% made by the intestine. Normally, excretion depends on plasma levels over a wide range of urate concentrations, from less than 1 mg/min at plasma urate below 5 mg/dl to more than 5 mg/min at plasma urate above 15 mg/dl. Upper limits of normal urate excretion have been established at 750 mg daily for women and 800 mg for men, but the reference range should be normalized for body weight or size. As mentioned before, less than 10% of urate filtered at the glomerulus is excreted in the urine, because of an efficient tubular reabsorption.

Because only a negligible fraction of urate is protein bound, virtually all of it is filtered at the glomerulus and as many as 8-9 g daily are delivered to the renal tubules. The tubular handling is rather complex, as it consists of a three-phase process which starts with reabsorption of the majority of the filtered urate, followed by tubular secretion and final by a more distal post-secretory reabsorption. The sequential interplay between these three phases is influenced by a number of factors, but basically depends on lumen-to-cell and cell-to-peritubular space gradients of urate concentrations. This issue has been object of much controversy, because of the difficulty to separate the single phases of urate renal handling. Most of the results were based on studies using drugs known to interfere with either reabsorption (i.e. sulfinpyrazone and probenecid) or secretion (pyrazinamide) by which, however, the relative contribution of early or post-secretory reabsorption remained unclear. The recent suggestion that pyrazinamide could act by facilitating reabsorption rather than inhibiting secretion of urate, might advocate reconsideration of this complex matter.⁴

Hyperuricemia

Hyperuricemia is the presence of high levels of uric acid in the blood. Hyperuricemia may occur because of decreased excretion. Hyperuricemia may also occur from increased production, or a combination of the two mechanisms. Underexcretion accounts for the majority of cases of hyperuricemia. Overproduction accounts for only a minority of patients presenting with hyperuricemia. The prevalence rate of asymptomatic hyperuricemia in the general population is estimated at 2-13%.

Consumption of purine-rich diets is one of the main causes of hyperuricemia. Other dietary causes are ingestion of high protein and fat, and starvation. Starvation results in the body metabolizing its own muscle mass for energy, in the process releasing purines into the bloodstream. Purine bases composition of foods varies. Foods with higher content of purine bases adenine and hypoxanthine are suggested to be more potent in exacerbating hyperuricemia.

Humans lack uricase, an enzyme which degrades uric acid. Increased levels predispose for gout and (if very high) renal failure. Apart from normal variation (with a genetic component), tumor lysis syndrome produces extreme levels of uric acid, mainly leading to renal failure. The Lesch-Nyhan syndrome is also associated with extremely high levels of uric acid. The Metabolic syndrome often presents with hyperuricemia, while a hyperuricemic syndrome is also common in Dalmatian dogs.

A uricase described herein and a pH increasing agent, alone or in combination with another agent, e.g., another agent described herein, can be used to treat hyperuricemia.

A uricase described herein and a hydrogen peroxide degrading enzyme (e.g., peroxidase or catalase), alone or in combination with another agent, e.g., another agent described herein, can be used to treat hyperuricemia.

Asymptomatic Hyperuricemia

Asymptomatic hyperuricemi_is the term for an abnormally high serum urate level, without gouty arthritis or nephrolithiasis. Hyperuricemia is defined as a serum urate concentration greater than 7 mg per dL (416 μmol per L), the approximate level at which urate is supersaturated in plasma.⁵

Although gouty arthritis characteristically occurs in patients with hyperuricemia, it is incorrect to equate hyperuricemia with clinical gout. Researchers from the Normative Aging Study followed 2,046 initially healthy men for 15 years by taking serial measurements of serum urate levels.⁶ The five-year cumulative incidence rates of gouty arthritis were 2.0 percent for a serum urate level of 8.0 mg per dL (475 μmol per L) or lower, 19.8 percent for urate levels from 9.0 to 10.0 mg per dL (535 to 595 μmol per L) and 30 percent for a serum urate level higher than 10 mg per dL (595 μmol per L).²

Hyperuricemia predisposes patients to both gout and nephrolithiasis, but therapy is occasionally not warranted in the asymptomatic patient. Recognizing hyperuricemia in the asymptomatic patient, however, provides the physician with an opportunity to modify or correct underlying acquired causes of hyperuricemia.¹

A uricase described herein and a pH increasing agent, alone or in combination with another agent, e.g., another agent described herein, can be used to treat asymptomatic hyperuricemia.

A uricase described herein and a hydrogen peroxide degrading enzyme (e.g., peroxidase or catalase), alone or in combination with another agent, e.g., another agent described herein, can be used to treat asymptomatic hyperuricemia.

Hyperuricosuria

Hyperuricosuria is defined as urinary excretion of uric acid greater than 800 mg/d in men and greater than 750 mg/d in women. This may be due to either excess dietary intake of purine-rich foods or endogenous uric acid overproduction. Hyperuricosuria may be associated with hyperuricemia.

A uricase described herein and a pH increasing agent, alone or in combination with another agent, e.g., another agent described herein, can be used to treat hyperuricosuria.

A uricase described herein and a hydrogen peroxide degrading enzyme (e.g., peroxidase or catalase), alone or in combination with another agent, e.g., another agent described herein, can be used to treat hyperuricosuria.

Gout

Gout is a condition that results from crystals of uric acid depositing in tissues of the body. Gout is characterized by an overload of uric acid in the body and recurring attacks of joint inflammation (arthritis). Chronic gout can lead to deposits of hard lumps of uric acid in and around the joints, decreased kidney function, and kidney stones.

Gout has the unique distinction of being one of the most frequently recorded medical illnesses throughout history. It is often related to an inherited abnormality in the body's ability to process uric acid. Uric acid is a breakdown product of purines, that are part of many foods. An abnormality in handling uric acid can cause attacks of painful arthritis (gout attack), kidney stones, and blockage of the kidney filtering tubules with uric acid crystals, leading to kidney failure. On the other hand, some patients may only develop elevated blood uric acid levels (hyperuricemia) without having arthritis or kidney problems. The term “gout” commonly is used to refer to the painful arthritis attacks.¹

Gouty arthritis is usually an extremely painful attack with a rapid onset of joint inflammation. The joint inflammation is precipitated by deposits of uric acid crystals in the joint fluid (synovial fluid) and joint lining (synovial lining). Intense joint inflammation occurs as white blood cells engulf the uric acid crystals and release chemicals of inflammation, causing pain, heat, and redness of the joint tissues. ²

According to MedicineNet, “While hyperuricemia may indicate an increased risk of gout, the relationship between hyperuricemia and gout is unclear. Many patients with hyperuricemia do not develop gout, while some patients with repeated gout attacks have normal or low blood uric acid levels. Among the male population in the United States, approximately ten percent have hyperuricemia. However, only a small portion of those with hyperuricemia will actually develop gout. Current standard urate lowering therapies include allopurinol, probenicide, puricase, etc. However, they have limited effectiveness and are not always well tolerated. In addition, not all patients respond to these therapies.

Allopurinol and its metabolite oxypurinol will act as effective competitive inhibitors of xanthine-oxidase. In case of massive uricosuria, allopurinol must be used at a higher than usual dosage, that is 600-900 mg/daily, to accomplish a more complete reduction of urate production.

Excess serum accumulation of uric acid can lead to a type of arthritis known as gout (gouty arthritis). Elevated (serum uric acid) levels (hyperuricemia) can result from high intake of purine-rich foods, high fructose intake (regardless of fructose's low Glycemic Index (GI) value) and/or impaired excretion by the kidneys. Saturation levels of uric acid in blood may result in one form of kidney stones when the uric acid crystallizes in the kidney. These uric acid stones are radiolucent and so do not appear on an abdominal x-ray. Their presence must be diagnosed by ultrasound for this reason. Some patients with gout eventually get uric kidney stones.

The small joint at the base of the big toe is the most common site of an acute gout attack. Other joints that can be affected include the ankles, knees, wrists, fingers, and elbows. Acute gout attacks are characterized by a rapid onset of pain in the affected joint followed by warmth, swelling, reddish discoloration, and marked tenderness.

A uricase described herein and a pH increasing agent, alone or in combination with another agent, e.g., another agent described herein, can be used to treat gout.

A uricase described herein and a hydrogen peroxide degrading enzyme (e.g., peroxidase or catalase), alone or in combination with another agent, e.g., another agent described herein, can be used to treat gout.

Tumor Lysis Syndrome

Tumor lysis syndrome (TLS) is a group of metabolic complications that can occur after treatment of cancer usually lymphomas and leukemias, and sometimes even without treatment. These complications are caused by the break-down products of dying cancer cells and include hyperkalemia, hyperphosphatemia, hyperuricemia and hyperuricosuria, hypocalcemia, and consequent acute uric acid nephropathy and acute renal failure.

Cause and risk factors. The most common tumors associated with this syndrome are poorly differentiated lymphomas, such as Burkitt's lymphoma, and leukemias, such as acute lymphoblastic leukemia (ALL) and acute myeloid leukemia (AML). Other cancers (such as melanoma) have also been associated with TLS but are less common.

Usually, the precipitating medication regimen includes combination chemotherapy, but those patients with lymphoma and ALL can be affected with steroid treatment alone, and sometimes without any treatment—in this case the condition is referred to as “spontaneous tumor lysis syndrome”.

Symptoms and Pathogenesis

Hyperkalemia. Potassium is mainly an intracellular ion. High turnover of tumor cells leads to spill of potassium into the blood. Symptoms usually do not manifest until levels are high (>7 mmol/dL) [normal 3.5-5.0 mmol/dL] and they include cardiac conduction abnormalities (can be fatal) and severe muscle weakness or paralysis.

Hyperphosphatemia. Like potassium, phosphates are also predominantly intracellular. Hyperphosphatemia causes acute renal failure in tumor lysis syndrome, because of deposition of calcium phosphate crystals in the renal parenchyma.

Hypocalcemia. Because of the hyperphosphatemia, calcium is precipitated to form calcium phosphate, leading to hypocalcemia. Symptoms of hypocalcemia include (but are not limited to): tetany, seizures, mental retardation/dementia, parkinsonian (extrapyramidal) movement disorders, papilledema, emotional instability/agitation/anxiety, and myopathy.

Hyperuricemia and Hyperuricosuria. Acute uric acid nephropathy (AUAN) due to hyperuricosuria has been a dominant cause of acute renal failure but with the advent of effective treatments for hyperuricosuria, AUAN has become a less common cause than hyperphosphatemia. Two common conditions related to excess uric acid, gout and uric acid nephrolithiasis, are not features of tumor lysis syndrome.

Pretreatment spontaneous tumor lysis syndrome. This entity is associated with acute renal failure due to uric acid nephropathy prior to the institution of chemotherapy and is largely associated with lymphomas and leukemias. The important distinction between this syndrome and the post-chemotherapy syndrome is that spontaneous TLS is not associated with hyperphosphatemia. One suggestion for the reason of this is that the high cell turnover rate leads to high uric acid levels through nucleobase turnover but the tumor reuses the released phosphate for growth of new tumor cells. In post-chemotherapy TLS, tumor cells are destroyed and no new tumor cells are being synthesized.

Diagnosis. TLS should be suspected in patients with large tumor burden who develop acute renal failure along with hyperuricemia (>15 mg/dL) or hyperphosphatemia (>8 mg/dL). (Most other acute renal failure occurs with uric acid <12 mg/dL and phosphate <6 mg/dL). Acute uric acid nephropathy is associated with little or no urine output. The urinalysis may show uric acid crystals or amorphous urates. The hypersecretion of uric acid can be detected with a high urine uric acid−creatinine ratio>1.0, compared to a value of 0.6-0.7 for most other causes of acute renal failure.

In 2004, Cairo and Bishop defined a classification system for tumour lysis syndrome.

Laboratory tumor lysis syndrome: abnormalitiy in two or more of the following and occurs within 3 days before or 7 days after chemotherapy.

uric acid >8 mg/dL or 25% increase

potassium >6 meq/L or 25% increase

phosphate >4.5 mg/dL or 25% increase

calcium <7 mg/dL or 25% decrease

Clinical tumor lysis syndrome: laboratory tumor lysis syndrome plus one or more of the following: increase serum creatinine (1.5 times upper limit of normal), cardiac arrhythmia or sudden death, and seizure.

A grading scale (0-5) is used depending on the presence of lab TLS, serum creatinine, arrhythmias, or seizures.

Treatment. Treatment is first targeted at the specific metabolic disorder. For example:

Acute renal failure prior to chemotherapy. Since the major cause of acute renal failure in this setting is uric acid build-up, therapy consists of rasburicase to wash out excessive uric acid crystals as well as a loop diuretic and fluids. Sodium bicarbonate should not be given at this time. If the patient does not respond, hemodialysis may be instituted, which is very efficient in removing uric acid, with plasma uric acid levels falling about 50% with each six hour treatment.

Acute renal failure after chemotherapy. The major cause of acute renal failure in this setting is hyperphosphatemia, and the main therapeutic means is hemodialysis. Forms of hemodialysis used include continuous arteriovenous hemodialysis (CAVHD), continuous venovenous hemofiltration (CVVH), or continuous venovenous hemodialysis (CVVHD).

A uricase described herein and a pH increasing agent, alone or in combination with another agent, e.g., another agent described herein, can be used to treat tumor lysis syndrome.

A uricase described herein and a hydrogen peroxide degrading enzyme (e.g., peroxidase or catalase), alone or in combination with another agent, e.g., another agent described herein, can be used to treat tumor lysis syndrome.

Lesch-Nyhan Syndrome

Lesch-Nyhan syndrome (LNS), also known as Nyhan's syndrome, is a rare, inherited disorder caused by a deficiency of the enzyme hypoxanthine-guanine phosphoribosyltransferase (HGPRT). LNS is an X-linked recessive disease: the gene is carried by the mother and passed on to her son. LNS is present at birth in baby boys. Patients have severe mental and physical problems throughout life. The lack of HGPRT causes a build-up of uric acid in all body fluids, and leads to problems such as severe gout, poor muscle control, and moderate mental retardation, which appear in the first year of life. A striking feature of LNS is self-mutilating behaviors, characterized by lip and finger biting, that begin in the second year of life. Abnormally high uric acid levels can cause sodium uric acid crystals to form in the joints, kidneys, central nervous system and other tissues of the body, leading to gout-like swelling in the joints and severe kidney problems. Neurological symptoms include facial grimacing, involuntary writhing, and repetitive movements of the arms and legs similar to those seen in Huntington's disease. The direct cause of the neurological abnormalities remains unknown. Because a lack of HGPRT causes the body to poorly utilize vitamin B12, some boys may develop a rare disorder called megaloblastic anemia.

The symptoms caused by the buildup of uric acid (arthritis and renal symptoms) respond well to treatment with drugs such as allopurinol that reduce the levels of uric acid in the blood. The mental deficits and self-mutilating behavior do not respond to treatment. There is no cure, but many patients live to adulthood. LNS is rare, affecting about one in 380,000 live births.

A uricase described herein and a pH increasing agent, alone or in combination with another agent, e.g., another agent described herein, can be used to treat Lesch-Nyhan syndrome.

A uricase described herein and a hydrogen peroxide degrading enzyme (e.g., peroxidase or catalase), alone or in combination with another agent, e.g., another agent described herein, can be used to treat Lesch-Nyhan syndrome.

Uric Acid Nephrolothiasis

Uric acid stones account for about 5 to 10% of all kidneys stones in Western countries and Japan. The stones can be composed of uric acid alone or admixed with calcium oxalate. Sex distribution indicates a male to female ratio of more than one, which tends to diminish in the post-menopausal age. While it is widely agreed that uric acid supersaturation accounts for the occurrence and clinical severity of uric acid stones, the incidence and role of altered uric acid elimination in this setting has not been confirmed. Several studies reporting on metabolic evaluation of kidney stones, have failed to find hyperuricosuria as the main risk factor. Similarly, the association between hyperuricemia and uric acid nephrolithiasis is far from being clear. Earlier reports indicated that uric acid nephrolithiasis had a prevalence significantly higher among gouty patients than normal individuals. However, a consistent portion of stones in gouty patients was found to be calcium-containing stones. The latter observation was subsequently assumed as the basis to hypothesize a causal role of uricosuria in the pathogenesis of calcium oxalate stones. Paradoxically, whereas the role of elevated uric acid in urine is of lesser importance in case of uric acid nephrolithiasis, it may represent a risk factor for calcium oxalate stone formation. Coe et al. speculated that hyperuricosuria can cause calcium oxalate nephrolithiasis by promoting the formulation of monosodium urate or uric acid crystals, thereby acting as seed crystals for calcium oxalate or absorbing macromolecular inhibitors of calcium oxalate crystallization.³ They observed that urine from calcium oxalate stone-formers were supersaturated with respect to monosodium urate or uric acid more frequently than other stone formers or normal individuals. Based on this, allopurinol was challenged in treatment of hyperuricosuric calcium stone formers and was found to be effective in the prevention f stone recurrences, which decreased from 0.26 per patient per year in the placebo group to 0.12 in the allopurinol group.

Kidney stones, also called renal calculi, are solid concretions (crystal aggregations) of dissolved minerals in urine; calculi typically form inside the kidneys or bladder. The terms nephrolithiasis and urolithiasis refer to the presence of calculi in the kidneys and urinary tract, respectively.

The formation of uric acid stones is associated with conditions that cause high blood uric acid levels, such as gout, leukemias/lymphomas treated by chemotherapy (secondary gout from the death of leukemic cells), and acid/base metabolism disorders where the urine is excessively acid resulting in uric acid precipitation.

A uricase described herein and a pH increasing agent, alone or in combination with another agent, e.g., another agent described herein, can be used to treat kidney stones and nephrolithiasis, e.g., kidney stones and/or nephrolithiasis caused by, or associated with, elevated uric acid concentrations.

A uricase described herein and a hydrogen peroxide degrading enzyme (e.g., peroxidase or catalase), alone or in combination with another agent, e.g., another agent described herein, can be used to treat kidney stones and nephrolithiasis, e.g., kidney stones and/or nephrolithiasis caused by, or associated with, elevated uric acid concentrations.

Renal Disease

Renal failure or kidney failure is a situation in which the kidneys fail to function adequately. It is divided in acute and chronic forms; either form may be due to a large number of other medical problems.

Biochemically, it is typically detected by an elevated serum creatinine. In the science of physiology, renal failure is described as a decrease in the glomerular filtration rate. When the kidneys malfunction, problems frequently encountered are abnormal fluid levels in the body, deranged acid levels, abnormal levels of potassium, calcium, phosphate, hematuria (blood in the urine) and (in the longer term) anemia. Long-term kidney problems have significant repercussions on other diseases, such as cardiovascular disease.

Renal failure can broadly be divided into two categories: acute renal failure and chronic kidney disease.

The type of renal failure (acute vs. chronic) is determined by the trend in the serum creatinine. Other factors which may help differentiate acute and chronic kidney disease include the presence of anemia and the kidney size on ultrasound. Long-standing, i.e. chronic, kidney disease generally leads to anemia and small kidney size.

Acute renal failure: Acute renal failure (ARF) is a rapidly progressive loss of renal function, generally characterized by oliguria (decreased urine production, quantified as less than 400 mL per day in adults, less than 0.5 mL/kg/h in children or less than 1 mL/kg/h in infants); body water and body fluids disturbances; and electrolyte derangement. An underlying cause must be identified to arrest the progress, and dialysis may be necessary to bridge the time gap required for treating these fundamental causes. ARF can result from a large number of causes.

Chronic kidney disease: Stage 5 Chronic Kidney Disease (CKD) can either develop slowly and show few initial symptoms, be the long term result of irreversible acute disease or be part of a disease progression. There are many causes of CKD. The most common cause is diabetes mellitus. Stage 1 CKD is mildly diminished renal function, with few overt symptoms. Stage 5 CKD is a severe illness and requires some form of renal replacement therapy (dialysis or kidney transplant).

Acute on chronic renal failure: Acute renal failure can be present on top of chronic renal failure. This is called acute-on-chronic renal failure (AoCRF). The acute part of AoCRF may be reversible and the aim of treatment, as with ARF, is to return the patient to their baseline renal function, which is typically measured by serum creatinine. AoCRF, like ARF, can be difficult to distinguish from chronic renal failure, if the patient has not been monitored by a physician and no baseline (i.e., past) blood work is available for comparison.

A uricase described herein and a pH increasing agent, alone or in combination with another agent, e.g., another agent described herein, can be used to treat renal disease caused by, or associated with, elevated uric acid concentrations.

A uricase described herein and a hydrogen peroxide degrading enzyme (e.g., peroxidase or catalase), alone or in combination with another agent, e.g., another agent described herein, can be used to treat renal disease caused by, or associated with, elevated uric acid concentrations.

Cardiovascular Disease

A growing body of evidence suggests that both high blood levels of uric acid and gout increase the risk of heart disease, including heart attack.

A uricase described herein and a pH increasing agent, alone or in combination with another agent, e.g., another agent described herein, can be used to treat cardiovascular disease caused by, or associated with, elevated uric acid concentrations.

A uricase described herein and a hydrogen peroxide degrading enzyme (e.g., peroxidase or catalase), alone or in combination with another agent, e.g., another agent described herein, can be used to treat cardiovascular disease caused by, or associated with, elevated uric acid concentrations.

Diabetes

Studies have shown that high serum uric acid is associated with higher risk of type 2 diabetes independent of obesity, dyslipidemia, and hypertension.

A uricase described herein and a pH increasing agent, alone or in combination with another agent, e.g., another agent described herein, can be used to treat diabetes caused by, or associated with, elevated uric acid concentrations.

A uricase described herein and a hydrogen peroxide degrading enzyme (e.g., peroxidase or catalase), alone or in combination with another agent, e.g., another agent described herein, can be used to treat diabetes caused by, or associated with, elevated uric acid concentrations.

Metabolic Syndrome

Metabolic syndrome is a cluster of conditions that occur together, increasing the risk of heart disease, stroke and diabetes. Metabolic syndrome involves having several disorders related to metabolism at the same time, including: obesity; elevated blood pressure; an elevated level of triglycerides; a low level of high-density lipoprotein (HDL) cholesterol; high blood pressure and/or high insulin levels 1.

Hyperuricemia is associated with components of metabolic syndrome and it has been debated for a while to be a component of it. It has been shown in a recent study that fructose-induced hyperuricemia may play a pathogenic role in the metabolic syndrome. This agrees with the increased consumption of fructose-base drinks in recent decades and the epidemic of diabetes and obesity.

A uricase described herein and a pH increasing agent, alone or in combination with another agent, e.g., another agent described herein, can be used to treat metabolic syndrome caused by, or associated with, elevated uric acid concentrations.

A uricase described herein and a hydrogen peroxide degrading enzyme (e.g., peroxidase or catalase), alone or in combination with another agent, e.g., another agent described herein, can be used to treat metabolic syndrome caused by, or associated with, elevated uric acid concentrations.

Hypertension

Stage 1 hypertension is a systolic pressure ranging from 140 to 159 or a diastolic pressure ranging from 90 to 99. The most severe hypertension, stage 2 hypertension is a systolic pressure of 160 or higher or a diastolic pressure of 100 or higher.

Excessive pressure on the artery walls can damage your vital organs. The higher your blood pressure and the longer it goes uncontrolled, the greater the damage.

Uncontrolled high blood pressure can lead to:

Damage to your arteries. This can result in hardening and thickening of the arteries (atherosclerosis), which can lead to a heart attack or other complications. An aneurysm also is possible.

Heart failure. To pump blood against the higher pressure in your vessels, your heart muscle thickens. Eventually, the thickened muscle may have a hard time pumping enough blood to meet your body's needs, which can lead to heart failure.

A blocked or ruptured blood vessel in your brain. This can lead to stroke.

Weakened and narrowed blood vessels in your kidneys. This can prevent these organs from functioning normally

Thickened, narrowed or torn blood vessels in the eyes. This can result in vision loss.

Metabolic syndrome. This syndrome is a cluster of disorders of your body's metabolism—including elevated waist circumference, high triglycerides, low high-density lipoprotein (HDL) cholesterol, high blood pressure and high insulin levels. If you have high blood pressure, you're more likely to have other components of metabolic syndrome. The more components you have, the greater your risk of developing diabetes, heart disease or stroke.

As arterial hypertension quite often coexists with gout, treating it with losartan, an angiotensin II receptor antagonist, might have an additional beneficial effect on uric acid plasma levels. This way losartan can offset the negative side-effect of thiazides (a group of diuretics used for high blood pressure) on uric acid metabolism in patients with gout.

A uricase described herein and a pH increasing agent, alone or in combination with another agent, e.g., another agent described herein, can be used to treat hypertension caused by, or associated with, elevated uric acid concentrations.

A uricase described herein and a hydrogen peroxide degrading enzyme (e.g., peroxidase or catalase), alone or in combination with another agent, e.g., another agent described herein, can be used to treat hypertension caused by, or associated with, elevated uric acid concentrations.

Uricase

Uricase (UO, urate oxidase, urate: oxygen oxidoreductase (E.C. 1.7.3.3)). Uricase degrades the poorly soluble uric acid (˜11 mg/100 ml H₂O), into the more soluble product allantoin (˜147 mg/100 ml H₂O). However, humans, chimpanzees, orangutans, and gibbons have a non-sense codon inserted into this gene that results in the synthesis of a short (10 amino acid) fragment devoid of enzymatic activity. As a result, high concentrations of uric acid can occur in humans, and when saturation is reached, this precipitates out of solution and collects in tissues and joints, causing a profound inflammatory reaction resulting in pain and loss of function and permanent damage to joints, connective tissues, and kidneys.

In some methods, uricase is obtained from Candida utilis (Amano Enzyme, Japan). This protein is a 120 kDa protein that consists of four identical monomers. This enzyme is stable between pH 7.0 to 10.0, with an optimal pH at 8.5.

Isoforms of uricase and glycoforms of those isoforms, recombinant forms, enzymes with similar function are included within this definition. Uricase from animals, plants, bacteria and fungi are encompassed by the term, including uricase from bacteria and fungi, such as Arthobacter globiformis, Bacillus thermocatenulatus, Bacillus fastidiosus, Bacillussp, TB-90 Microbacterium sp, Aspergillus flavus, Aspergillus terreus, Aspergillus nidulas, Aspergillus niger Trichoderma sp. from leaves of chickpea, Cicer arietimum, broad bean, Vicia faba major, wheat, Triticum aestivum, Neurospora crassa, Rhizopus oryzae, Candida tropicalis, Candida utilis, soybean.

Uricase catalyzes the oxidation of uric acid (urate) to 5-hydroxyisourate:

Uric acid+O₂+H₂O→5-hydroxyisourate+H₂O₂→allantoin+CO₂

Uricase is a homotetrameric enzyme containing four identical active sites situated at the interfaces between its four subunits. Uricase from A. flavus is made up of 301 residues and has a molecular weight of 33,438 daltons. It is unique among the oxidases in that it does not require a metal atom or an organic co-factor for catalysis.

Humans do have the gene for uricase but it is nonfunctional. As a result, uric acid is the final step in the catabolism of purines in humans.

Excessive concentrations of uric acid accumulated in the blood stream can lead to gout. Uricase has been formulated for the treatment of acute hyperuricaemia as a protein drug (non-proprietary drug name rasburicase) in patients receiving chemotherapy. A PEGylated form of uricase (PEG-uricase) is in clinical development for treatment of chronic hyperuricemia in patients with treatment-failure gout.

Uricase is available from many sources, including Aspergillus flavus (e.g., Aspergillus flavus enzyme PDB 1R4U, EC 1.7.3.3), Streptomyces cyanogenus, Pseudomonas aeruginosa, Vigna unguiculata, rust Puccinia recondite, Bacillus fastidiosus.

Additional sources include: active strains of micro-organisms which are either bacteria, especially those of the genus Bacillus, or fungi especially those which belong to the genera Mucor, Rhizopus, Absidia, Fusarium, Alternaria, Penicillium, Aspergillus, Cephalosporium, Stemphylium and Macrosporum, or yeasts, especially of the genus Geotrichum. These genera belong to the orders of eubacteriales, actinomycetales, mucorales, moniliales, spheriales and endomycetales. The uricase may also be obtained using bacteria of the genera Pseudomonas, Clostridium, Micrococcus and Bacterium, fungi of the genus Neurospora and yeasts of the genera Saccharomyces and Torula (Candida). Uricase can be prepared using bacteria and fungi belonging to the species Streptomyces cellulosae and Strept. sulfureus, Bacillus megatherium, B. subtilis and B. cereus, Aspergillus flavus, Asp. oryzae, Asp. tamarii, Asp. terricola, Asp. luchuensis, Asp. niger, Asp. sydowi, Asp. nidulans, Asp. wentii, Asp. fonsecaeus, Asp. clavatus, Asp. ustus, Asp. terreus and Asp. ochraceus, Penicillium frequentans, Pen. granulatum, Pen. griseum, Pen. canescens, Pen. spinulosum, Pen. thomii, Pen. waksmani, Pen. raistrickii, Pen. expansum, Pen. purpurescens, Pen. funiculosum, Pen. spiculisporum, Pen. velutinum, Pen. purpurogenum, Pen. lilacinum, Pen. rubrum, Cephalosporium, Alternaria tenuis, Fusarium solani, Fus. moniliforme, Fus. coeruleum, Fus. oxysporum and Fus. orthoceras, Stemphylium macrosporoideum, Macrosporium apiospermum, Absidia glauca, Mucor mucedo, Mucor hiemalis and Mucor racemosus, Rhizopus arrhizus and of the class Basidiomycetes, as well as the yeast Geotrichum candidum. Suitable micro-organisms are given below. In this list each name is followed in brackets by the registered number of the strain deposited in the American Type Culture Collection (A.T.C.C.).
a. Bacteria: Streptomyces cellulosae Krainsky (21184), Streptomyces sulphureus Krainsky (21185), Bacillus megatherium of Barry (21180), Bacillus megatherium of Barry (21181), Bacillus subtilis Cohn (21183) and Bacillus cereus Frankland (21182).
b. Fungi other than yeasts: Aspergillus flavus (20037), A.f. (20038), A.f. (20039), A.f. (20040), A.f. (20041), A.f. (20042), A.f. (20043), A.f. (20044), A.f. (20045), A.f. (20046), A.f. (20047), A.f. (20048), Aspergillus oryzae Cohn (20049), A.o. Cohn (20050), A.o. Cohn (20051), A.o. Cohn (20052), A.o. Cohn (20053), Aspergillus tamarii Kita (20054), Asp. terricola Marchal (20055), Asp. luchuensis Inui (20056), Asp. Niger Van Tieghem (20057), Asp. sydowi Bainier and Sartory (20058), Asp. nidulans Wint (20059), Asp. wentii Wehmer (20060), Asp. fonsecaeus Bainier (20061), Asp. clavatus Blochwitz (20062), Asp. ustus Thom and Church (20063), Asp. terreus Thom (20064), Asp. ochraceus Wilhelm (20065), Penicillium frequentans Westling (20066), Pen. granulatum (20067), Pen. griseum Thom (20068), Pen. canescens Sopp (20069), Pen. spinulosum Thom (20070), Pen. thomii Maire (20071), Pen. waksmani Zaleski (20072), Pen. raistrickii (Smith) (20073), Pen. expansum Thom (20074), Pen. purpurescens Sopp (20075), Pen. funiculosum Thom (20076), Pen. spiculisporum Lehman (20082), Pen. velutinum (Van Beyma) (20081), Pen. purpurogenum Stoll (20077), Pen. spinulosum Thom (20078), Pen. lilacinum Thom (20079), Pen. rubrum Stoll (20080), Cephalosporium sp. Corda (20083), Alternaria tenuis Nees (20084), A. tenuis (20085), Fusarium solani Appel & Wollenweber (20086), Fus. moniliforme Sheldon (20087), Fus. coeruleum Sacchardo (20088), Fus. oxysporum Schelechtendahl (20089), Stemphylium macrosporoideum Saccardo (20090), Macrosporium apiospermum (20091), Fusarium orthoceras Appel & Wollenweber (20092), Absidia glauca+(Hagem) (20093), Mucor mucedo Brefeld (20094), Mucor hiemalis Wehmer (20095), Mucor racemosus Fresnius (20096), Rhizopus arrhizus Fisher (20097), Basidiomycete sp. (20098).
c. Yeasts: Geotrichum candidum (20099)

Uricase may also be purchased from commercial purveyors, such as, e.g., Sigma Aldrich. Methods to isolate uricase from a natural source are previously described, for example, in U.S. Pat. No. 3,620,923. Ohe and Watanabe, J. Biochem. 89:1769-1776 (1981). Specific examples of uricase sequences are provided herein.

Recombinant Uricase. In some instances, recombinant uricase encompasses or is encoded by sequences from a naturally occurring uricase sequence. Further, the uricase of the disclosure may comprise an amino acid sequence that is homologous or substantially identical to a naturally occurring sequence or other sequence described herein. Also, uricases encoded by a nucleic acid that is homologous or substantially identical to a naturally occurring uricase-encoding nucleic acid are provided.

Polypeptides referred to herein as “recombinant” are polypeptides which have been produced by recombinant DNA methodology, including those that are generated by procedures which rely upon a method of artificial recombination, such as the polymerase chain reaction (PCR) and/or cloning into a vector using restriction enzymes. “Recombinant” polypeptides are also polypeptides having altered expression, such as a naturally occurring polypeptide with recombinantly modified expression in a cell, such as a host cell.

In one embodiment, uricase is recombinantly produced from a nucleic acid that is homologous to a uricase nucleic acid sequence described herein, and sometimes it is modified, e.g., to increase or optimize recombinant production in a heterologous host.

In some embodiments, uricase for use in the present compositions and methods is encoded by a nucleic acid that comprises a nucleic acid sequence that is at least 40%, 50%, 60%, 70%, 75%, 80%, 85%, 90%, 91%, 92%, 93%, 94%, 95%, 96%. 97%, 98%, 99%, or 100% identical to a nucleic acid described herein and that encodes a protein that possesses a function of a uricase described herein, e.g., the encoded protein can catalyze the oxidation of uric acid (urate) to 5-hydroxyisourate.

In some embodiments, uricase for use in the present compositions has an amino acid sequence that comprises an amino acid sequence that is at least 40%, 50%, 60%, 70%, 75%, 80%, 85%, 90%, 91%, 92%, 93%, 94%, 95%, 96%, 97%, 98%, 99%, or 100% identical to an amino acid described herein and the protein possesses a function of a uricase described herein, e.g., the protein can catalyze the oxidation of uric acid (urate) to 5-hydroxyisourate.

In some embodiments, uricase for use in the present compositions and methods is encoded by a nucleic acid that hybridizes under stringent conditions (e.g., under high stringency) to the complement of a nucleic acid described herein, and the nucleic acid encodes a protein that possesses a function of a uricase described herein, e.g., the encoded protein can catalyze the oxidation of uric acid (urate) to 5-hydroxyisourate.

Uricase polypeptides useful in the compositions and methods herein may be expressed in a host cell, such as a host cell comprising a nucleic acid construct that includes a coding sequence for a uricase polypeptide or a functional fragment thereof. A suitable host cell for expression of uricase may be yeast, bacteria, fungus, insect, plant, or mammalian cell, for example, or transgenic plants, transgenic animals or a cell-free system. Preferably, a host cell is capable of post-translationally modifying (e.g., glycosylating) the uricase polypeptide if necessary, capable of disulfide linkages, capable of secreting the uricase, and/or capable of supporting multimerization of uricase polypeptides. Preferred host cells include, but are not limited to E. coli (including E. coli Origami B and E. coli BL21), Pichia pastoris, Saccharomyces cerevisiae, Schizosaccharomyces pombe, Bacillus subtilis, Aspergillus, Sf9 cells, Chinese hamster ovary (CHO), 293 cells (human embryonic kidney), and other human cells. Also transgenic plants, transgenic animals including pig, cow, goat, horse, chicken, and rabbit are suitable hosts for production of uricase.

For recombinant production of uricase, a host or host cell should comprise a construct in the form of a plasmid, vector, phagemid, or transcription or expression cassette that comprises at least one nucleic acid encoding a uricase or a functional fragment thereof. A variety of constructs are available, including constructs which are maintained in single copy or multiple copy, or which become integrated into the host cell chromosome. Many recombinant expression systems, components, and reagents for recombinant expression are commercially available, for example from Invitrogen Corporation (Carlsbad, Calif.); U.S. Biological (Swampscott, Mass.); BD Biosciences Pharmingen (San Diego, Calif.); Novagen (Madison, Wis.); Stratagene (La Jolla, Calif.); and Deutsche Sammlung von Mikroorganismen and Zellkulturen GmbH (DSMZ), (Braunschweig, Germany).

Recombinant expression of uricase is optionally controlled by a heterologous promoter, including a constitutive and/or inducible promoter. Promoters such as, e.g., T7, the alcohol oxidase (AOX) promoter, the dihydroxy-acetone synthase (DAS) promoters, the Gal 1,10 promoter, the phosphoglycerate kinase promoter, the glyceraldehyde-3-phosphate dehydrogenase promoter, alcohol dehydrogenase promoter, copper metallothionein (CUP1) promoter, acid phosphatase promoter, CMV and promoters polyhedrin are also appropriate. The particular promoter is selected based on the host or host cell. In addition, promoters that are inducible by methanol, copper sulfate, galactose, by low phosphate, by alcohol, e.g., ethanol, for example, may also be used and are well known in the art.

A nucleic acid that encodes uricase may optionally comprise heterologous sequences. For example, a secretion sequence is included at the N-terminus of a uricase polypeptide in some embodiments. Signal sequences such as those from a Mating Factor, BGL2, yeast acid phosphatase (PHO), xylanase, alpha amylase, from other yeast secreted proteins, and secretion signal peptides derived from other species that are capable of directing secretion from the host cell may be useful. Similarly other heterologous sequences such as linkers (e.g., comprising a cleavage or restriction endonuclease site) and one or more expression control elements, an enhancer, a terminator, a leader sequence, and one or more translation signals are within the scope of this description. These sequences may optionally be included in a construct and/or linked to the nucleic acid that encodes uricase. Unless otherwise specified, “linked” sequences can be directly or indirectly associated with one another.

Similarly, an epitope or affinity tag such as Histidine, HA (hemagglutinin peptide), maltose binding protein, AVITAG®, FLAG, or glutathione-S-transferase may be optionally linked to the uricase polypeptide. A tag may be optionally cleavable from the uricase after it is produced or purified. A skilled artisan can readily select appropriate heterologous sequences, for example, match host cell, construct, promoter, and/or secretion signal sequence.

Uricase homologs or variants differ from a uricase reference sequence by one or more residues. Structurally similar amino acids can be substituted for some of the specified amino acids, for example. Structurally similar amino acids include: (I, L and V); (F and Y); (K and R); (Q and N); (D and E); and (G and A). Deletion, addition, or substitution of amino acids is also encompassed by the uricase homologs described herein. Such homologs and variants include (i) polymorphic variants and natural or artificial mutants, (ii) modified polypeptides in which one or more residues is modified, and (iii) mutants comprising one or more modified residues.

A uricase polypeptide or nucleic acid is “homologous” (or is a “homolog”) if it is at least 40%, 50%, 60%, 70%, 75%, 80%, 85%, 90%, 91%, 92%, 93%, 94%, 95%, 96%. 97%, 98%, 99%, or 100% identical to a reference sequence. If the homolog is not identical to the reference sequence, it is a “variant.” A homolog is “substantially identical” to a reference uricase sequence if the nucleotide or amino acid sequence of the homolog differs from the reference sequence (e.g., by truncation, deletion, substitution, or addition) by no more than 1, 2, 3, 4, 5, 8, 10, 20, or 50 residues, and retains (or encodes a polypeptide that retains) a function of uricase, e.g., retains the ability to catalyze the oxidation of uric acid to 5-hydroxyisourate. Fragments of a uricase may be homologs, including variants and/or substantially identical sequences. By way of example, homologs may be derived from various sources of uricase, or they may be derived from or related to a reference sequence by truncation, deletion, substitution, or addition mutation. Percent identity between two nucleotide or amino acid sequences may be determined by standard alignment algorithms such as, for example, Basic Local Alignment Tool (BLAST) described in Altschul et al., J. Mol. Biol., 215:403 410 (1990), the algorithm of Needleman et al., J. Mol. Biol., 48:444 453 (1970), or the algorithm of Meyers et al., Comput. Appl. Biosci. 4:11 17 (1988). Such algorithms are incorporated into the BLASTN, BLASTP, and “BLAST 2 Sequences” programs (reviewed in McGinnis and Madden, Nucleic Acids Res. 32:W20-W25, 2004). When utilizing such programs, the default parameters can be used. For example, for nucleotide sequences the following settings can be used for “BLAST 2 Sequences”: program BLASTN, reward for match 2, penalty for mismatch 2, open gap and extension gap penalties 5 and 2 respectively, gap x_dropoff 50, expect 10, word size 11, filter ON. For amino acid sequences the following settings can be used for “BLAST 2 Sequences”: program BLASTP, matrix BLOSUM62, open gap and extension gap penalties 11 and 1 respectively, gap x_dropoff 50, expect 10, word size 3, filter ON. The amino acid and nucleic acid sequences for uricase that are appropriate for use in the methods described herein may include homologous, variant, or substantially identical sequences.

The uricase of this disclosure, e.g., of the compositions and methods described herein may be purified uricase. A “purified” polypeptide or protein is substantially free of cellular material or other contaminating proteins from the cell or tissue source from which the protein is derived, or substantially free from chemical precursors or other chemicals when chemically synthesized. “Substantially free” means that the protein of interest in the preparation is at least 10% pure. In an embodiment, the preparation of the protein has less than about 30%, 20%, 10% and more preferably 5% (by dry weight), of a contaminating component (e.g., a protein not of interest, chemical precursors, and so forth). When the protein or biologically active portion thereof is recombinantly produced, it is also preferably substantially free of culture medium, i.e., culture medium represents less than about 20%, more preferably less than about 10%, and most preferably less than about 5% of the volume of the protein preparation.

Uricase crystals. Uricase of this disclosure, e.g., of the compositions and methods described herein may be crystallized. Examples of uricase crystals are provided herein.

Formulation of uricase. Uricase may be enterically coated to make it stable to low pH and proteolytic cleavage.

Administration: Uricase can be orally administered together or in sequence with a pH increasing agent. Preferably, the agent increases pH to 5 or above. pH increasing agents may be a carbonate, bicarbonate, or their salt forms such as sodium bicarbonate, magnesium carbonate, potassium carbonate, ammonium carbonate, anti-acids and proton pump inhibitors, etc. Uricase can be orally administered together or in sequence with a hydrogen peroxide degrading enzyme (e.g., peroxidase or catalase).

Protection of Uricase

An approach to increase the stability of uricase (e.g., relative to an unstabilized uricase in the same conditions), e.g., in an acidic environment, e.g., in acidic conditions, e.g., in the gastrointestinal tract is by use of a polyionic reagent. This approach can be used instead of, or in combination with, other approaches described herein, e.g., the use of a pH increasing agent.

To protect uricase against acidic pH of the stomach, a number of polyionic reagents can be used, for example, aromatic sulfonates and their derivatives which will bind to protein by forming ion pairs between negatively charged reagent and positively charged protein at acidic pH to form insoluble precipitate can be used. In addition, they form the hydrophobic interaction between the aromatic groups of the protein and the reagent, thus providing extra protection against acidic pH.

The protein can also be protected against acid by covalently attaching the protein to a polymer that will form insoluble precipitate at acidic pH and will encapsulate the protein inside the precipitated polymer. For example, Alginate, Eudragit L100-55, Pectin, Polyacrylic acid, poly hyaluronic acid and sodium carboxymethyl cellulose can be used for this purpose. This method also can be used against proteolytic degradation of uricase in the gut.

A number of other methods such as chemical modification of uricase at amino acid residues such as tyrosine, phenylalanine, tryptophan, arginine and lysine may also help in protecting the uricase against proteolytic cleavage. In addition, covalently attaching a protease inhibitor such as Bowman-Birk inhibitor or pepstatin to the uricase will help protect uricase against proteases.

Examples of polyionic coating materials include: PSS: poly(Sodium 4-styrenesulfonate), PAA: poly Acrylic acid sodium salt, PMG: poly(methylene-co-guanidine) hydrochloride, DS: dextran sulfate, PMA: poly(methyl acrylate), and PVS: Polyvinylsiloxane. One or more coatings may be applied and/or one or more different polyionic coating materials may be used, e.g., one or more PMG and PAA coatings may be applied to a uricase preparation, e.g., uricase srystals.

pH Increasing Agents

Another approach to increase the stability of uricase, e.g., in an acidic environment, e.g., in acidic consitions, e.g., in the gastrointestinal tract is to administer the uricase with a pH increasing agent. This approach can be used instead of, or in combination with, other approaches described herein, e.g., the use of polyelectrolyte coatings.

A pH increasing agent can be administered with uricase. The pH increasing agent and uricase can be administered, e.g., by an enteral route (e.g., orally). For example, the pH increasing agent can increase the pH of the stomach such that the uricase is more stable in that environment. For example, the pH increasing agent can raise stomach pH to above about 5 (e.g., increases the pH to about 5, about 5.5, about 6, about 6.5, about 7, about 7.5, or higher).

pH-increasing agents include a carbonate, bicarbonate, or salt forms thereof, such as sodium bicarbonate, magnesium carbonate, potassium carbonate, ammonium carbonate; anti-acids and proton pump inhibitors, etc.

In preferred embodiments, the pH increasing agent does not denature the uricase. For example, if the pH increasing agent is combined with uricase prior to administration to a subject or if the pH increasing agent is administered at the same time- or close in time (e.g., the pH increasing agent is administered within (before or after) about 5, about 10, about 15, about 20, about 25, about 30 minutes, about 60 minutes, about 120 minutes of when the uricase is administered)—to the time at which uricase is administered to a subject, the pH increasing agent does not denature the uricase.

In preferred embodiments, the pH increasing agent does not abolish the activity of the uricase. For example, if the pH increasing agent is combined with uricase prior to administration to a subject or if the pH increasing agent is administered at the same time- or close in time (e.g., the pH increasing agent is administered within (before or after) about 5, about 10, about 15, about 20, about 25, about 30 minutes, about 60 minutes, about 120 minutes of when the uricase is administered)—to the time at which uricase is administered to a subject, the pH increasing agent does not abolish the activity of the uricase. For example, the uricase retains at least about 5%, about 10%, about 15%, about 20%, about 25%, about 30%, about 35%, about 40%, about 45, about 50%, about 55%, about 60%, about 65%, about 70%, about 75%, about 80%, about 85%, about 90%, about 95%, or about 100% of the activity level the uricase had prior to its exposure to the pH increasing agent.

In some embodiments, the pH increasing agent increases uricase activity (e.g., in vitro or in vivo (e.g., in a subject's stomach)), as compared to the uricase activity in the absence of the pH increasing agent under the same conditions (e.g., in vitro or in vivo). For example, uricase activity can be increased by at least about 5%, about 10%, about 15%, about 20%, about 25%, about 30%, about 35%, about 40%, about 45, about 50%, about 55%, about 60%, about 65%, about 70%, about 75%, about 80%, about 85%, about 90%, about 95%, or about 100%, as compared to the uricase activity in the absence of the pH increasing agent under the same conditions.

The carbonate ion is a polyatomic anion with the empirical formula CO₃²⁻. A carbonate salt forms when a positively charged ion attaches to the negatively charged oxygen atoms of the ion, forming an ionic compound. Many carbonate salts are insoluble in water at standard temperature and pressure, with solubility constants of less than 1×10⁻⁸. Exceptions include sodium, potassium and ammonium carbonates.

In aqueous solution, carbonate, bicarbonate, carbon dioxide, and carbonic acid exist together in a dynamic equilibrium. In strongly basic conditions, the carbonate ion predominates, while in weakly basic conditions, the bicarbonate ion is prevalent. In more acid conditions, aqueous carbon dioxide, CO₂(aq), is the main form, which, with water, H₂O, is in equilibrium with carbonic acid—the equilibrium lies strongly towards carbon dioxide. Thus, sodium carbonate is basic, sodium bicarbonate is weakly basic, while carbon dioxide itself is a weak acid.

Bicarbonate is an alkaline, and a vital component of the pH buffering system of the body (maintaining acid-base homeostasis). 86%-90% of CO₂ in the body is converted into carbonic acid (H₂CO₃), which can quickly turn into bicarbonate (HCO₃⁻).

With carbonic acid as the central intermediate species, bicarbonate, in conjunction with water, hydrogen ions, and carbon dioxide forms this buffering system which is maintained at the volatile equilibrium required to provide prompt resistance to drastic pH changes in both the acidic and basic directions.

Bicarbonate also acts to regulate pH in the small intestine. It is released from the pancreas in response to the hormone secretin to neutralize the acid chyme entering the duodenum from the stomach

Sodium bicarbonate (baking soda) is thought to work by raising blood pH (lowering blood acidity).

Examples of carbonate salts include sodium carbonate, potassium carbonate, calcium carbonate, magnesium carbonate, lithium carbonate, and ammonium carbonate.

Examples of bicarbonate salts include sodium bicarbonate, potassium bicarbonate, ammonium bicarbonate, calcium bicarbonate, magnesium bicarbonate, and lithium bicarbonate.

An antacid is a substance, generally a base, which counteracts stomach acidity. In other words, antacids are stomach acid neutralizers. Antacids perform a neutralization reaction, i.e. they buffer gastric acid, raising the pH to reduce acidity in the stomach. Examples of antacids include: Aluminium hydroxide (AMPHOJEL®, ALTERNAGEL®); Magnesium hydroxide (PHILLIPS'® Milk of Magnesia); Aluminum hydroxide and magnesium hydroxide (MAALOX®, MYLANTA®); Aluminum carbonate gel (BASALJEL®); Calcium carbonate (ALCALAK®, TUMS®, QUICK-EZE®, RENNIE®, TITRALAC®, ROLAIDS®); Sodium bicarbonate (Bicarbonate of soda, ALKASELTZER®); Hydrotalcite (Mg₆Al₂(CO₃)(OH)₁₆ 4(H₂O); TALCID®); Bismuth subsalicylate (PEPTO-BISMOL®); and Magaldrate+Simethicone (PEPSIL).

Proton pump inhibitors (or “PPI”s) are a group of drugs whose main action is pronounced and long-lasting reduction of gastric acid production. Proton pump inhibitors act by blocking the hydrogen/potassium adenosine triphosphatase enzyme system (the H⁺/K⁺ ATPase, or more commonly just gastric proton pump) of the gastric parietal cell. The proton pump is the terminal stage in gastric acid secretion, being directly responsible for secreting H⁺ ions into the gastric lumen, making it an ideal target for inhibiting acid secretion. Examples of proton pump inhibitors include: Omeprazole (brand names: LOSEC®, PRILOSEC®, ZEGERID®); Lansoprazole (brand names: PREVACID®, ZOTON®, INHIBITOL®); Esomeprazole (brand names: NEXIUM®); Pantoprazole (brand names: PROTONIX®, SOMAC®, PANTOLOC®, PANTOZOL®, ZURCAL®); Rabeprazole (brand names: RABECID®, ACIPHEX®, PARIET®).

In the compositions and methods described herein, carbonate or a carbonate salt (e.g., sodium carbonate, potassium carbonate, calcium carbonate, magnesium carbonate, lithium carbonate, or ammonium carbonate), bicarbonate or a bicarbonate salt (e.g., sodium bicarbonate, potassium bicarbonate, ammonium bicarbonate, calcium bicarbonate, magnesium bicarbonate, or lithium bicarbonate), an antacid, or a proton pump inhibitor can be used in combination with a uricase.

Hydrogen Peroxide Degrading Enzyme

A hydrogen peroxide degrading enzyme may be used with uricase, or with uricase and a pH increasing agent, e.g., in the methods described herein. The hydrogen peroxide degrading enzyme may be present in a composition that contains uricase or that contains uricase and a pH increasing agent. For example, uricase can be stabilized as described herein, e.g., by encapsulation, and used with the hydrogen peroxide degrading enzyme.

Examples of hydrogen peroxide degrading enzymes include peroxidase and catalase.

Peroxidase. A peroxidase (e.g., enzyme peroxidase) may be used with uricase, or with uricase and a pH increasing agent, e.g., in the methods described herein. The peroxidase may be present in a composition that contains uricase or that contains uricase and a pH increasing agent. For example, uricase can be stabilized as described herein, e.g., by encapsulation, and used with the peroxidase.

Peroxidases are a large family of enzymes. A majority of peroxidase protein sequences can be found in the PeroxiBase database. Peroxidases typically catalyze a reaction of the form:

ROOR′+electron donor (2 e−)+2H+→ROH+R′OH

For many of these enzymes, the optimal substrate is hydrogen peroxide, but others are more active with organic hydroperoxides such as lipid peroxides. Peroxidases can contain a heme cofactor in their active sites, or redox-active cysteine or selenocysteine residues.

Examples of peroxidases include haloperoxidases, glutathione peroxidases, and myeloperoxidases. In some aspects of this disclosure, the peroxidase is catalase.

Catalase. Catalase may be used with uricase, or with uricase and a pH increasing agent, e.g., in the methods described herein. Catalase may be present in a composition that contains uricase or that contains uricase and a pH increasing agent. For example, uricase can be stabilized as described herein, e.g., by encapsulation, and used with catalase.

Catalase is a common enzyme found in nearly all living organisms. Its functions include catalyzing the decomposition of hydrogen peroxide (a product of reaction catalyzed by uricase) to water and oxygen. For example, uricase enzyme activity produces hydrogen peroxide, which can be detrimental to a subject. Catalase can process the hydrogen peroxide to less detrimental compounds (water and oxygen).

Catalase has one of the highest turnover rates of all enzymes; one molecule of catalase can convert millions of molecules of hydrogen peroxide to water and oxygen per second.

Catalase is a tetramer of four polypeptide chains, each over 500 amino acids long.

It contains four porphyrin heme (iron) groups that allow the enzyme to react with the hydrogen peroxide.

The reaction of catalase in the decomposition of hydrogen peroxide is:

2 H₂O₂→2 H₂O+O₂

Catalase can be obtained from a commercial source, e.g., Worthington Biochemical Corporation, Sigma. Assays for catalase activity are described, e.g., by Worthington Biochemical Corporation's Enzyme Manual. See also Beers, R., and Sizer, I.: J Biol Chem 195, 133 (1952); Gregory, E., and Fridovich, I.: Anal Biochem 58, 57 (1974); Haining, J., and Legan, J.: Anal Biochem 45, 469 (1972); and Kroll, R., Frears, E., and Bayliss, A, J Appl Bacteriol 66, 209 (1989).

Catalase is known in the art and is available from many sources, e.g., b Bos taurus, Homo sapiens, Saccharomyces cerevisiae, Proteus mirabilis, Helicobacter pylori; Enterococcus faecalis; Micrococcus lysodeikticus; Pseudomonas syringae.

Additional Compounds for Combination Therapy

An additional agent, e.g., another agent that can be used for treating disorders associated with elevated uric acid concentrations in a subject, such as a xanthine-oxidase inhibitor, and/or a uricosuric, can be used in combination with a uricase described herein, e.g., in the methods described herein, e.g., to treat a disorder described herein. The additional agent can optionally be present in a composition in combination with the uricase. Alternatively, the additional agent can be administered in combination with the uricase but present in a separate composition. The routes of administration for the agents can be the same or can differ.

Xanthine-Oxidase Inhibitors: The enzyme xanthine oxidase catalyzes the oxidation of hypoxanthine to xanthine and can further catalyze the oxidation of xanthine to uric acid. In humans, xanthine oxidase is normally found in the liver and not free in the blood. During severe liver damage, xanthine oxidase is released into the blood, so a blood assay for xanthine oxidase is a way to determine if liver damage has happened. Because xanthine oxidase is a metabolic pathway for uric acid formation, a xanthine oxidase inhibitor can be used in the treatment of gout.

Allopurinol is a xanthine-oxidase inhibitor, widely used in the prevention of attacks of gout, and well tolerated. It is safe to use in patients with renal impairment and uric acid stones. Marketed forms of allopurinol include: Zyloprim, Allohexyl, Allosig, Progout, and Zyloric.

Other xanthine oxidase inhibitors include: 2-(3-cyano-4-isobutoxyphenyl)-4-methyl-5-thiazolecarboxylic acid (TEI-6720); febuxostat (a non-purine inhibitor; 2-[3-cyano-4-isobutoxyphenyl]-4-methylthiazole-5-carboxylic acid), oxypurinol, and pteridylaldehyde.

Uricosurics: Uricosuric medications are substances that increase the excretion of uric acid in the urine, thus reducing uric acid concentrations in plasma. Generally, this effect is achieved by action on the distal renal tubule. Uricosurics often are used in the treatment of gout. Examples include probenecid and sulfinpyrazone. By decreasing plasma uric acid levels, these drugs decrease the deposition of crystals in joints, eventually decreasing inflammation and thereby mitigating the pain of gout. Losartan also has uricosuric properties, though that is not its main use.

Sulfinpyrazone is an uricosuric used to treat gout. It is less widely used than allupurinol, and must not be used in patients with renal impairment, or a high uric acid excretion rate.

Probenecid, a uricosuric drug that promotes the excretion of uric acid in urine, is also commonly prescribed—often in conjunction with colchicine. The drug fenofibrate (which is used in treating hyperlipidemia) also exerts a beneficial uricosuric effect.

Other Agents: These additional agents can also be used in combination with the uricase described herein.

Ethylenediaminetetraacetic acid, a chelator of lead, has successfully increased uric acid excretion. This should be an advantageous treatment for those people whose gout was caused by lead poisoning. Care should be taken to increase intake of trace essential elements since chelation often remove these elements also.

Gout can be triggered by the same agents that cause potassium losses such as fasting, surgery, and potassium losing diuretics. A potassium deficiency can increase uric acid levels in the blood. So potassium supplements should be advantageous to treat gout.

Acetazolamide, is a carbonic anhydrase inhibitor is also used to treat hyperuricemia. It may be sold under the name Diamox.

Long-term coffee consumption may be associated with a lower risk of gout.

PEG-uricase (e.g., PURICASE® (pegloticase) from Savient), a polyethylene glycol (“PEG”) conjugate of recombinant porcine uricase (uricase), which breaks down the uric acid deposits is being studied in clinical trials for the treatment of severe, treatment-refractory gout in the United States.

Pharmaceutical Compositions

Uricase (or uricase and a pH increasing agent; or uricase and hydrogen peroxide degrading enzyme (e.g., peroxidase or catalase)) can be formulated as a pharmaceutical composition for administration to a subject, e.g., to treat a disorder described herein. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge, S. M., et al. (1977) J. Pharm. Sci. 66:1-19).

Pharmaceutical formulation is a well-established art, and is further described, e.g., in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} Ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X).

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically, compositions for the uricase described herein are in a form for oral administration.

In one embodiment, the uricase (or uricase and a pH increasing agent; or uricase and a hydrogen peroxide degrading enzyme (e.g., peroxidase or catalase)) is formulated with excipient materials, such as sodium chloride, sodium dibasic phosphate heptahydrate, sodium monobasic phosphate, and a stabilizer. It can be provided, for example, in a buffered solution at a suitable concentration and can be stored at 2-8° C.

Such compositions can be administered by a parenteral mode (e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and infrasternal injection and infusion.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the uricase (or uricase and a pH increasing agent; or uricase and a hydrogen peroxide degrading enzyme (e.g., peroxidase or catalase)) may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York (1978).

A uricase (or uricase and a pH increasing agent; or uricase and a hydrogen peroxide degrading enzyme (e.g., peroxidase or catalase)) can be modified, e.g., with a moiety that improves its stabilization and/or retention in circulation, e.g., in blood, serum, or other tissues, e.g., by at least 1.5, 2, 5, 10, or 50 fold. The modified uricase can be evaluated to assess whether it can reach sites of disease, e.g., articular cartilage of joints, tendons and surrounding tissues.

For example, the uricase (or uricase and a pH increasing agent; or uricase and hydrogen peroxide degrading enzyme (e.g., peroxidase or catalase)) can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used.

For example, the uricase (or uricase and a pH increasing agent; or uricase and hydrogen peroxide degrading enzyme (e.g., peroxidase or catalase)) can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides.

When the uricase (or uricase and a pH increasing agent; or uricase and hydrogen peroxide degrading enzyme (e.g., peroxidase or catalase)) is used in combination with an additional agent (e.g., a xanthine-oxidase inhibitor, and/or an uricosuric), the agents can be formulated separately or together. The agents can be formulated or otherwise used in a synergistically effective amount, or in an additively effective amount. It is also possible to use one or both of the agents in amounts less than would be used for mono-therapy. As a result of each agent being used at a lower amount than in mono-therapy, side effects and/or toxicity of the agent(s) can be reduced. For example, the respective pharmaceutical compositions can be mixed, e.g., just prior to administration, and administered together or can be administered separately, e.g., at the same or different times.

Upon improvement of a patient's condition, a maintenance dose of a compound, composition or combination of this disclosure may be administered, if necessary. Subsequently, the dosage or frequency of administration, or both, may be reduced, e.g., to about ½ or ¼ or less of the dosage or frequency of administration, as a function of the symptoms, to a level at which the improved condition is retained when the symptoms have been alleviated to the desired level, treatment should cease. Subjects may, however, require intermittent treatment on a long-term basis upon any recurrence of disease symptoms.

Administration

The uricase (or uricase and a pH increasing agent; or uricase and a hydrogen peroxide degrading enzyme (e.g., peroxidase or catalase)) can be administered to a subject, e.g., a human subject, by a variety of methods. In many cases, the enteral route is used (e.g., given directly into the gastrointestinal tract, e.g., oral administration). For some applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and infrasternal injection. In some cases, administration may be directly to a site of the disorder, e.g., articular cartilage of joints, tendons and surrounding tissues. For combination therapies, the agents being combined for the therapy do not need to be administered by the same route.

The route and/or mode of administration of the enzyme can also be tailored for the individual case, e.g., by monitoring the subject, e.g., using tomographic imaging, e.g., to visualize a joint.

The uricase (or uricase and a pH increasing agent) can be administered as a fixed dose, or in a mg/kg dose. The dose can also be chosen to reduce or avoid production of antibodies against the uricase. Dosage regimens are adjusted to provide the desired response, e.g., a therapeutic response or a combinatorial therapeutic effect. Generally, doses of uricase (and optionally a second agent) can be used in order to provide a subject with the agent in bioavailable quantities. For example, doses in the range of 0.1-100 mg/kg, 0.5-100 mg/kg, 1 mg/kg-100 mg/kg, 0.5-20 mg/kg, 0.1-10 mg/kg, or 1-10 mg/kg can be administered. Other doses can also be used.

Dosage unit form or “fixed dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent. Single or multiple dosages may be given. Alternatively, or in addition, the antibody may be administered via continuous infusion.

A uricase (or uricase and a pH increasing agent; or uricase and a hydrogen peroxide degrading enzyme (e.g., peroxidase or catalase)) dose can be administered, e.g., at a periodic interval over a period of time (a course of treatment) sufficient to encompass at least 2 doses, 3 doses, 5 doses, 10 doses, or more, e.g., once or twice daily, or about one to four times per week, or preferably weekly, biweekly, monthly, e.g., for between about 1 to 12 weeks, preferably between 2 to 8 weeks, or between about 3 to 7 weeks, or for about 4, 5, or 6 weeks, or about 1, 2, 3 or more years. Factors that may influence the dosage and timing required to effectively treat a subject, include, e.g., the severity of the disease or disorder, formulation, route of delivery, previous treatments, the general health and/or age of the subject, and other diseases present. Moreover, treatment of a subject with a therapeutically effective amount of a compound can include a single treatment or, preferably, can include a series of treatments. Animal models can also be used to determine a useful dose, e.g., an initial dose or a regimen.

If a subject is at risk for developing gout or other uric acid-associated disorder described herein, the uricase (or uricase and a pH increasing agent; or uricase and hydrogen peroxide degrading enzyme (e.g., peroxidase or catalase)) can be administered before the full onset of the disorder, e.g., as a preventative measure. The duration of such preventative treatment can be a single dosage of the uricase (or uricase and a pH increasing agent; or uricase and hydrogen peroxide degrading enzyme (e.g., peroxidase or catalase)) or the treatment may continue (e.g., multiple dosages). For example, a subject at risk for the disorder or who has a predisposition for the disorder may be treated with the uricase (or uricase and a pH increasing agent; or uricase and hydrogen peroxide degrading enzyme (e.g., peroxidase or catalase)) for days, weeks, months, or even years so as to prevent the disorder from occurring or fulminating.

A pharmaceutical composition may include a “therapeutically effective amount” of an agent described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects.

Devices and Kits for Therapy

Pharmaceutical compositions that include the uricase (or uricase and a pH increasing agent; or uricase and a hydrogen peroxide degrading enzyme (e.g., peroxidase or catalase)) can be administered with a medical device. The device can designed with features such as portability, room temperature storage, and ease of use so that it can be used in emergency situations, e.g., by an untrained subject or by emergency personnel in the field, removed from medical facilities and other medical equipment. The device can include, e.g., one or more housings for storing pharmaceutical preparations that include the uricase (or uricase and a pH increasing agent; or uricase and hydrogen peroxide degrading enzyme (e.g., peroxidase or catalase)), and can be configured to deliver one or more unit doses of the antibody. The device can be further configured to administer a second agent, e.g., a xanthine-oxidase inhibitor, an uricosuric, a pH increasing agent, or a hydrogen peroxide degrading enzyme (e.g., peroxidase or catalase), either as a single pharmaceutical composition that also includes the uricase or as separate pharmaceutical compositions.

For example, the pharmaceutical composition, or one of the agents of a combination therapy, can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other devices, implants, delivery systems, and modules are also known.

A uricase (or uricase and a pH increasing agent; or uricase and a hydrogen peroxide degrading enzyme (e.g., peroxidase or catalase)) can be provided in a kit. In one embodiment, the kit includes (a) a container that contains a composition that includes the uricase (or uricase and a pH increasing agent; or uricase and hydrogen peroxide degrading enzyme (e.g., peroxidase or catalase)), and optionally (b) informational material. The informational material can be descriptive, instructional, marketing or other material that relates to the methods described herein and/or the use of the agents for therapeutic benefit.

In some embodiments, the kit includes a first container that contains a composition that includes the uricase and a second container that includes the pH increasing agent.

In some embodiments, the kit includes a first container that contains a composition that includes the uricase and a second container that includes a hydrogen peroxide degrading enzyme (e.g., peroxidase or catalase).

In an embodiment, the kit also includes a second agent for treating a disorder described herein, e.g., a xanthine-oxidase inhibitor, an uricosuric. For example, the kit includes a first container that contains a composition that includes the uricase (or uricase and a pH increasing agent; or uricase and hydrogen peroxide degrading enzyme (e.g., peroxidase or catalase)), and a second container that includes the second agent.

As another example, the kit includes a first container that contains a composition that includes the uricase, a second container that includes the pH increasing agent, and optionally, a third container that includes an additional agent.

As another example, the kit includes a first container that contains a composition that includes the uricase, a second container that includes the hydrogen peroxide degrading enzyme (e.g., peroxidase or catalase), and optionally, a third container that includes an additional agent.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the uricase (or uricase and a pH increasing agent; or uricase and hydrogen peroxide degrading enzyme (e.g., peroxidase or catalase)), e.g., in a suitable dose, dosage form, or mode of administration (e.g., a dose, dosage form, or mode of administration described herein), to treat a subject who has had or who is at risk for a uric acid-associated disorder described herein. The information can be provided in a variety of formats, include printed text, computer readable material, video recording, or audio recording, or information that provides a link or address to substantive material, e.g., on the internet.

In addition to the uricase (or uricase and a pH increasing agent; or uricase and hydrogen peroxide degrading enzyme (e.g., peroxidase or catalase)), the composition in the kit can include other ingredients, such as a solvent or buffer, a stabilizer, or a preservative. The uricase (or uricase and a pH increasing agent; or uricase and hydrogen peroxide degrading enzyme (e.g., peroxidase or catalase)) can be provided in any form, e.g., liquid, dried or lyophilized form; preferably substantially pure and/or sterile. When the agents are provided in a liquid solution, the liquid solution preferably is an aqueous solution. When the agents are provided as a dried form, reconstitution generally is by the addition of a suitable solvent. The solvent, e.g., sterile water or buffer, can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the uricase (or uricase and a pH increasing agent; or uricase and hydrogen peroxide degrading enzyme (e.g., peroxidase or catalase)) and the second agent, e.g., in a desired ratio. For example, the kit includes a plurality of syringes, ampules, foil packets, blister packs, oral dosage forms (e.g., tablet, capsule, or pill), or medical devices, e.g., each containing a single combination unit dose. The containers of the kits can be air tight, waterproof (e.g., impermeable to changes in moisture or evaporation), and/or light-tight.

The kit optionally includes a device suitable for administration of the composition (or one or more agents of a combination therapy), e.g., a syringe or other suitable delivery device. The device can be provided pre-loaded with one or both of the agents or can be empty, but suitable for loading.

The following examples provide illustrative embodiments of the invention. One of ordinary skill in the art will recognize the numerous modifications and variations that may be performed without altering the spirit or scope of the present invention. Such modifications and variations are encompassed within the scope of the invention. The Examples do not in any way limit the invention.

### EXAMPLES

### Example 1

### Formulation of Uricase and pH Modifying Agent (Mixture 1)

In the studies described herein, uricase from Candida utilis (Amano, Japan), a 120 kDa protein that consists of four identical monomers, was used. This enzyme is stable between pH 7.0 to 10.0, with an optimal pH at 8.5.

Uricase (5-200 mg/daily, equal or larger than 2.5 u/mg) and a pH-modifying agent such as sodium bicarbonate (Sigma, US) were mixed with a regular rodent diet (5P75, Purina Lab Diet, US or A04, Safe, France) to formulate an uricase mixture 1. The formulated mixture was administered orally, daily to uricase deficient mice (Uox^{-/-}), a model with severe hyperuricemia and urate nephropathy.⁵

### Example 2

### Formulation of Uricase and pH Modifying Agent and Catalase (Mixture 2)

Uricase (5-200 mg/daily, equal or larger than 2.5 u/mg) and a pH-modifying agent such as sodium bicarbonate (Sigma, US) and catalase (Sigma, US) were mixed with a regular rodent diet (5P75, Purina Lab Diet, US or A04, Safe, France) to formulate an uricase mixture 2. The formulated mixture was administered orally, daily to uricase deficient mice (Uox^{-/-}), a model with severe hyperuricemia and urate nephropathy.⁵

### Example 3

### Assay for Estimation of Uricase Specific Activity

Uricase specific activity is measured using a modified enzymatic assay as described by Amano and Genzyme Inc. The consumption of uric acid is measured in the 50 mM boric acid assay buffer, pH 8.0 containing 0.13 mM uric acid as a substrate at 293 nm by spectrophotometer. One unit of enzyme is defined as the amount of enzyme required to degrade one micromole of uric acid per minute per 25° C. and pH 8.5.

### Example 4

### Measurement of the Uric Acid Concentration in Urine Samples

18-24 h urinary samples were collected in metabolic cages. Urine samples were stored at 20° C. until further analysis. Daily diuresis and multiple 24 h urine samples were collected and analyzed for urate levels. To measure concentration of uric acid in the urine, colorimetric uric acid assay kit, QuantiChrom was used (BioAssay System, CA). Data were analyzed statistically using Student's t-test.

### Example 5

### Measurement of the Uric Acid Concentration in Plasma Samples

Every five to seven days mice were bled by orbital bleeds and plasma samples were collected for uric acid measurement. The uric acid assay kit, QuantiChrom (BioAssay System, CA) was used for plasma uric acid estimation.

### Example 6

### Oral Therapy with Uricase Combined with pH Modifying Agent (Mixture 1) in Animal Model for Hyperuricemia and Urate Nephropathy

A total of 14 female and male mice (strain (Uox^{-/-}, C57BL/6J), Jackson laboratory, CA) were used in these experiments. Mice were randomly divided between a control group and three experimental groups. Mice weighed 20-25 grams and were less than 6 months of age.

Mice were acclimated for 7 days prior to treatment, housed 3-4 mice per cage and were fed diet (17% proteins, 11% fat, 53.5% carbohydrate, 5P75, Purina Lab Diet, US). After the acclimation period, mice were divided into two groups randomly divided between to control placebo group and 200 mg uricase mixture 1 based on their basal urinary oxalate. All mice were kept on allopurinol (Zyloprim, 5 mg/dL) during the breeding and post-weaning periods. To increase the severity of disease, allopurinol was removed one week before the study. Drinking water and food were provided to all mice ad libitum from the first day of treatment until the end of the study. The result is analyzed by unpaired two tail Student's t-test. Each group had n=7 mice.

Assessment of the efficacy of uricase combined with pH modifying agent: The efficacy of the enzyme therapy was monitored by plasma urate and urinary urate reduction. During the study plasma and urine samples were collected periodically for estimation of uric acid.

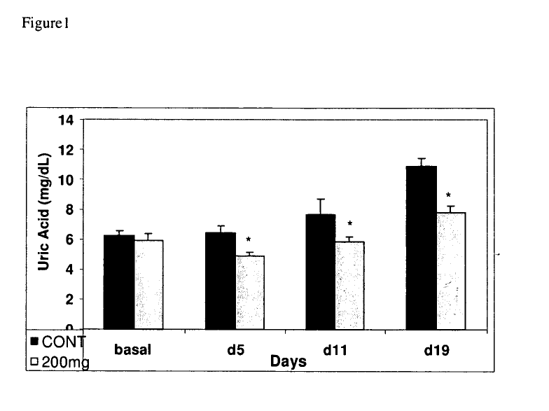

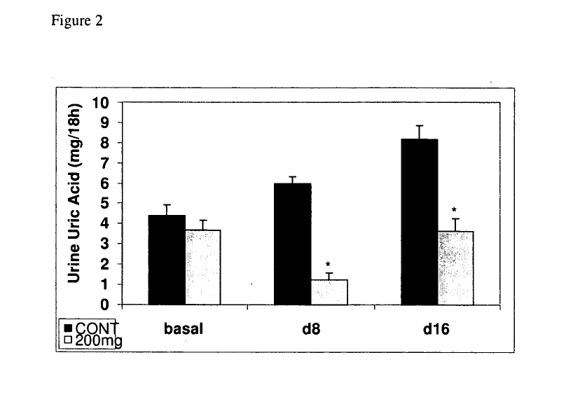

Summary of oral therapy with uricase combined with pH modifying agent (Mixture 1) in animal model for hyperuricemia and urate nephropathy. Oral administration of uricase mixture 1 (200 mg/day) to Uox^{-/-} mice resulted in significant reduction of plasma urate levels from day 5 of the treatment until the end of the study when compared with matched untreated control mice. A reduction up to 30% in plasma was demonstrated in the uricase group compared to placebo treated mice (FIG. 1). Similarly oral administration of uricase mixture (200 mg/day) to Uox^{-/-} mice resulted in significant reduction of urinary urate from day 8 of the treatment until the end of the study when compared with matched untreated control mice. A reduction in urine between 80% and 56% were recorded on the study days 8 and 16 respectively (FIG. 2). These results were analyzed by unpaired two tail Student's t-test. Each group had n=7 mice.

### Example 7

### Oral Therapy with Uricase Combined with pH Modifying Agent and Catalase (Mixture 2) in Animal Model for Hyperuricemia and Urate Nephropathy

In another experiment, the efficacy of uricase mixture 2 (200 mg/day) or placebo was monitored for 15 days in mice that had been treated with allopurinol (10 mg/d1 in the drinking water, ad libidum) continuously until being switched to therapy with uricase mixture 2. A total of 14 male and female (strain (Uox^{-/-}, C57BL/6J) mice were used in this experiment. Mice were randomly divided between a control group and one experimental group based on their basal plasma and urine uric acid levels. Mice weighed 20-25 grams and were less than 6 months of age. Mice were acclimated for 7 days prior to treatment to individual metabolic cages (Tecniplast USA Inc, Exton, Pa., USA), and were fed standard breeder diet (17% proteins, 11% fat, 53.5% carbohydrate, A04, Safe France).

Assessment of the efficacy of uricase combined with pH modifying agent: The efficacy of the enzyme therapy was monitored by plasma urate and urinary urate reduction. During the study plasma and urine samples were collected periodically for estimation of uric acid.

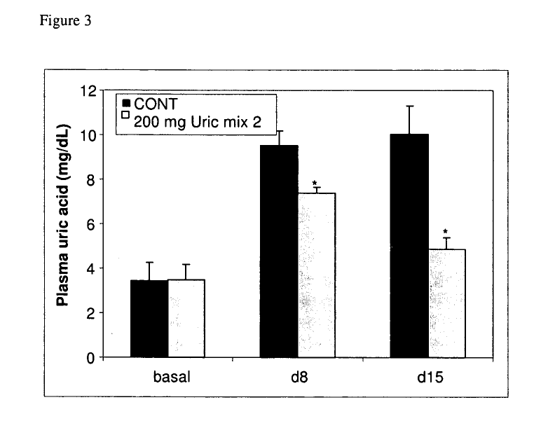

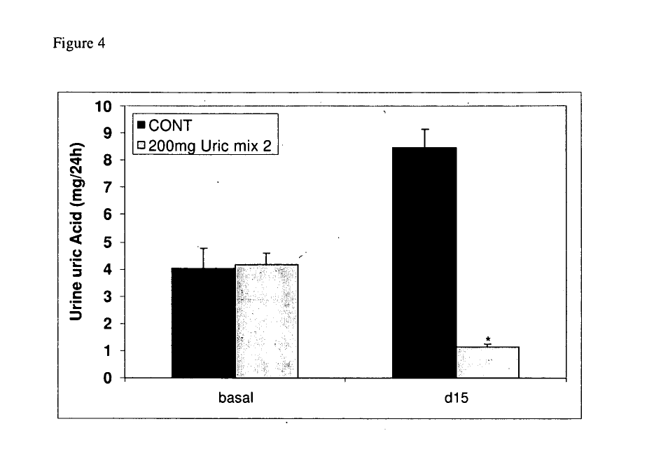

Summary of the oral therapy with uricase combined with pH modifying agent (Mixture 2) in animal model for hyperuricemia and urate nephropathy. Oral administration of uricase mixture 2 (200 mg/day) to Uox^{-/-} mice resulted in significant reduction of plasma urate levels from day 8 of the treatment until the end of the study when compared with matched untreated control mice. A reduction in hyperuricemia of 23% and 52% was demonstrated in the uricase mixture 2 group compared to placebo treated mice on day 8 and day 15, respectively (FIG. 3). Similarly oral administration of uricase mixture 2 (200 mg/day) to Uox^{-/-} mice resulted in significant reduction of urinary urate. We demonstrated hypruricosuria reduction of 87% in the mice from treatment group compared with matched untreated control mice (FIG. 4) These results were analyzed by unpaired two tail Student's t-test. Each group had n=5-7.

### Example 8

### Therapy with Oral Uricase with and without pH Modifying Agent (Mixture 2) in Animal Model for Hyperuricemia and Urate Nephropathy

In another experiment, the efficacy of uricase mixture 2 (100 mg/day) was compared to placebo treated mice and mice treated with uricase during 15 days. A total of 29 female and male mice (strain (Uox^{-/-/} C57BL/6J, Jackson laboratory, CA)/were used in these experiments. Mice were randomly divided between a control group and two experimental groups. Mice weighed 20-25 grams and were less than 6 months of age.

Mice were acclimated for 7 days prior to treatment, were housed 3-4 mice per cage and were fed diet (17% proteins, 11% fat, 54% carbohydrate, 5P75, Purina Lab Diet, US). After the acclimation period, mice were randomly divided in the groups based on their basal urinary oxalate, control placebo group, 100 mg uricase mixture 2 group and 100 mg uricase group. All mice were kept on allopurinol (Zyloprim, 5 mg/dL) during the breeding and post-weaning periods. To increase the severity of disease, allopurinol was removed 3 weeks before the study. Drinking water and food were provided to all mice ad libitum from the first day of treatment until the end of the study. For urine collection mice were placed in metabolic cages (Teckniplast USA Inc, Exton, Pa., USA), two times during the study, 2-3 mice/cage matched by group and gender. Urine was collected for 16 h.

Assessment of the efficacy of uricase combined with pH increasing agent: The efficacy of the enzyme therapy was monitored by urinary urate reduction. During the study plasma and urine samples were collected periodically for estimation of uric acid.

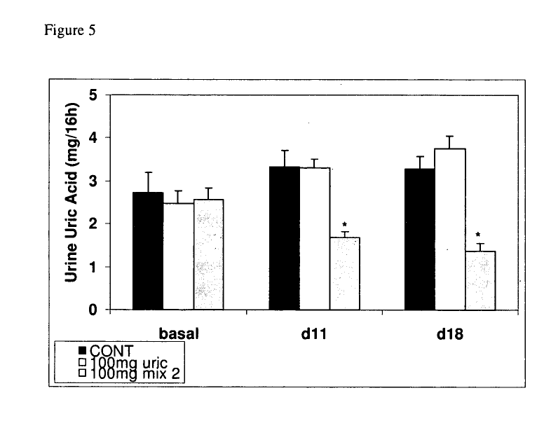

Summary of the oral therapy with uricase with and without pH modifying agent (Mixture 2) in animal model for hyperuricemia and urate nephropathy. As shown in FIG. 5, daily oral administration of uricase mixture 2 (100 mg/day) to Uox^{-/-} mice resulted in significant reduction of urinary urate only in the group of mice treated with 100 mg uricase with reducing (pH modifying) agent (mixture 2) when compared to control placebo group and group that was treated with uricase without pH modifying agent. We demonstrated hyperuricosuria reduction of 87% in the mice fed 100 mg uricase mixture 2 group compared with matched untreated control mice and mice fed 100 mg uricase. The result was analyzed by unpaired two tail Student's t-test. Each group had n=8-12 mice.

### Example 9

### Uricase Protein Sequence (SEQ ID NO:1). Candida utillis (Pichia Jadinii)

An exemplary uricase protein is as follows:

Accession Number: P78609

Pub Med: 8982864

Medline: 97137527

An exemplary uricase amino acid sequence is as follows:

(SEQ ID NO: 1)
MSTTLSSSTYGKDNVKFLKVKKDPQNPKKQEVMEATVTCLLEGGFDTSYT
EADNSSIVPTDTVKNTILVLAKTTEIWPIERFAAKLATHFVEKYSHVSGV
SVKIVQDRWVKYAVDGKPHDHSFIHEGGEKRITDLYYKRSGDYKLSSAIK
DLTVLKSTGSMFYGYNKCDFTTLQPTTDRILSTDVDATWVWDNKKIGSVY
DIAKAADKGIFDNVYNQAREITLTTFALENSPSVQATMFNMATQILEKAC
SVYSVSYALPNKHYFLIDLKWKGLENDNELFYPSPHPNGLIKCTVVRKEK
TKL

### Example 10

### Uricase Nucleotide Sequence Candida utillis (Pichia Jadinii)

An exemplary uricase nucleic acid sequence is as follows:

(SEQ ID NO: 2)
ATGTCAACAACGCTCTCATCATCCACCTACGGCAAGGACAACGTCAAGTT
CCTCAAGGTCAAGAAGGACCCGCAAAACCCAAAGAAGCAGGAGGTTATGG
AGGCCACCGTCACGTGTCTGCTTGAAGGTGGGTTCGACACCTCGTACACG
GAGGCTGACAACTCGTCCATCGTGCCAACAGACACCGTGAAGAACACCAT
TCTCGTGTTGGCAAAGACCACGGAGATTTGGCCAATTGAGAGATTTGCAG
CCAAGCTGGCCACGCACTTTGTTGAGAAGTACTCGCACGTCTCTGGCGTC
TCCGTCAAGATTGTCCAGGACAGATGGGTCAAGTACGCCGTTGATGGCAA
GCCACACGACCACTCTTTTATCCACGAAGGTGGTGAGAAGAGAATCACTG
ACCTGTACTACAAGAGATCCGGTGATTACAAGCTGTCGTCTGCCATCAAG
GACTTGACGGTGCTGAAGTCCACCGGCTCGATGTTCTACGGCTACAACAA
GTGTGACTTCACCACCTTGCAACCAACAACTGACAGAATCTTGTCCACCG
ACGTCGATGCCACCTGGGTTTGGGATAACAAGAAGATTGGCTCTGTCTAC
GACATCGCCAAGGCTGCAGACAAGGGAATCTTTGACAACGTTTACAACCA
GGCTAGAGAGATCACCTTGACCACCTTTGCTCTCGAGAACTCTCCATCTG
TGCAGGCCACGATGTTCAACATGGCTACTCAGATCTTGGAAAAGGCATGC
TCTGTCTACTCGGTTTCATACGCCTTGCCAAACAAGCACTACTTCCTCAT
TGACTTGAAATGGAAAGGTTTGGAGAACGACAACGAGTTGTTCTACCCAT
CTCCACATCCAAATGGGTTGATCAAGTGTACTGTTGTCCGTAAGGAGAAG
ACCAAGTTGTAG

### Example 11

### Oral Therapy with Uricase and a pH Increasing Agent Reduces Hyperuricosuria and Hyperuricemia in Mice Lacking Uricase (Uox^{-/-})

Introduction and Objectives: Elevated plasma uric acid, hyperuricemia, has been increasing in Western countries over the last decade and correlates well with an increase in prevalence of renal disease, gout, hypertension and metabolic syndrome. It occurs either as a result of excessive uric acid production or decrease in renal excretion of uric acid or both. Standard uric acid lowering therapies have limited effectiveness and are not always well tolerated. We posited a new approach for treatment of hyperuricemia, and tested it with the oral use of uricase and a pH increasing agent, that is stable and active in the pH- and protease-challenging environment of the intestine. We tested its efficacy on reduction of plasma and urinary uric acid in uricase deficient mice (Uox^{-/-}), a model with severe hyperuricemia and uric acid nephropathy. We hypothesized that a stable and active urate-specific enzyme will reduce the body pool of uric acid by first degrading intestinal uric acid and promoting a blood to lumen transepithelial gradient that will enhance enteric excretion and thereby reduce plasma uric acid levels. We compared efficacy of uricase and a pH increasing agent with allopurinol, a specific xantine oxidase inhibitor, as it is the most commonly used therapy for hyperuricemic patients and those hyperuricosuric patients with uric acid stones.

Methods: In the first experiment, for 3 weeks three different doses of uricase and a pH increasing agent (5, 50, and 200 mg/day, n=7) or placebo were mixed with the food and administered to Uox^{-/-} mice. In the second experiment the efficacy of uricase and a pH increasing agent (200 mg/day, n=7) was compared with a usual dose of allopurinol (100 mg/L in drinking water, −30 mg/kg/day n=7) for 15 days. All mice were kept during the breeding and post-weaning periods on allopurinol (10 mg/dL), except in the first experiment, when allopurinol was removed one week before the study.

Results: Hyperuricemia and uricosuria were reduced considerably upon daily oral administration of uricase and a pH increasing agent when compared to untreated controls in both experiments. In the first study, mice fed 200 mg of uricase and a pH increasing agent for 3 weeks had a mean overall reduction in urinary uric acid of 66% (2.45±0.77 mg/18 h vs. 7.1±0.49 mg/18 h., p<0.05) and plasma uric acid was reduced 26% (6.19±0.68 mg/dL vs. 8.35±0.68 mg/dL, p<0.05). In the same model we tested for 3 weeks, two doses of uricase and a pH increasing agent (50 and 5 mg/day). Compared with the untreated controls, we again demonstrated a constant reduction in uricosuria of 46% with the 50 mg dose (3.59±0.74 mg/24 h vs. 6.63±1.15 mg/24 h, p<0.05), while the lower dose had minimal or no effect, implying specificity of the drug action.

In the second experiment, treatment with 200 mg dose of uricase and a pH increasing agent reduced an elevated plasma uric acid 52% (4.86±0.55 mg/dL vs 10.06±1.23 mg/dL), similar to plasma uric acid reduction of 52% recorded in the group of mice treated with allopurinol (4.79±0.28 mg/dL vs. 10.06±1.23 mg/dL), each compared to control mice. Uricase and a pH increasing agent had a larger hypo-uricosuric effect then allopurinol, with a mean overall reduction in urinary uric acid of 76% compared to 38% recorded in allopurinol treated mice (p<0.05).

Conclusion: These studies indicate that uricase and a pH increasing agent has a potential as a new oral agent for treatment of hyperuricemia, hyperuricosuria, and related diseases.

### Example 12

### Procedure for Determining Uric Acid Levels in Plasma and Urine

### Introduction

Hyperuricemia is elevated uric acid levels in the blood which can predispose for gout and (if extremely high) renal failure. Uric acid which has limited solubility can accumulate to form insoluble stones in the kidneys. Humans lack urate oxidase (uricase) which breaks down uric acid to highly soluble allantoin, carbon dioxide and hydrogen peroxide.

We are working on developing uricase into an orally deliverable and stable, solid crystallized, formulated pill. In the gastrointestinal (GI) tract, uricase will work by breaking down uric acid, creating a concentration gradient between the bloodstream/kidney and the GI tract. This gradient will give rise to more uric acid being eliminated through the GI tract, thereby reducing uric acid levels in the bloodstream. Orally delivered uricase will be formulated to prevent absorption into the bloodstream thereby preventing any immunogenicity towards the drug.

All pre-clinical studies evaluating the efficacy of uricase in reducing uric acid levels in hyperuricemic (UoxKO) mice require an assay to measure uric acid levels in the plasma and urine. This example describes the assay procedure used for the determination of uric acid levels in mouse plasma and urine. A good assay needs to be reproducible along with being simple and easy, to allow analysis of large numbers of pre-clinical samples at one time.

### Principle

To measure the concentration of uric acid in plasma and urine samples we used the uric acid assay diagnostic kit purchased from BioAssay Systems. According to the manufacturer, this assay is designed to measure uric acid directly in serum/urine without any pretreatment of the samples. It is considered an improved method utilizing 2, 4, 6-tripyridyl-s-triazine that forms a blue colored complex with iron specifically in the presence of uric acid. The intensity of the color, measured at A_{590 nm}, is directly proportional to the uric acid concentration in the serum. The optimized formulation substantially reduces interference by substances in the raw samples.

### Equipment and Materials

### Equipment

96 well plates, Corning, Cat # 9017
96 well plate shaker,
UV micro-titer plate reader, Molecular Devices, Spectra Max Plus
96 well plate plastic covers
Disposable multichannel pipetter basin, Fisher Cat #13-681-100
Pipetting devices and accessories (e.g. 5 μl)

### Materials

Uric acid Assay kit (DIUA-250), BioAssay Systems, Cat #: DIUA-250
   composed of reagent A, B and C and 10 mg/dL uric acid standard and blank
Pre-clinical samples—urine and plasma
Mouse serum, Cat #: S7273-50 ml

### Procedure

### Reagent Procedure:

Shake Reagent C before use.
Mix sufficient working volume of Reagent B in a 1:1 ratio with 40 mM HCl prior to use.
Prepare enough working reagent by mixing 10 volumes of Reagent A, 1 volume of Reagent B+40 mM HCl solution and 1 volume of Reagent C. Prepare fresh and equilibrate to RT before use.

### Standards and Sample Preparation:

The standard curve is prepared from a 10 mg/dL uric acid standard supplied with kit, using fresh dilutions.

The standard curve is prepared as shown below in Table 1.

TABLE 1
Preparation of uric acid standard curve

| URIC ACID | WATER | 10 MG/DL URIC ACID STANDARD |
|---|---|---|
| (MG/DL) | (μL) | (μL) |
| 10 | 0 | 100 |
| 7.5 | 100 | 300 |
| 5.0 | 200 | 200 |
| 2.5 | 300 | 100 |
| 1 | 360 | 40 |

Procedure using 96-well plate:

Urine and plasma samples are thawed immediately prior to use. Urine samples can be thawed faster by placing tubes in a container with cold water to quicken the thawing time. Samples are vortexed for a few seconds and used neat or diluted in the water. For example for 20× dilution, 10 μL of urine is mixed with 190 μL water.

Plasma samples are mixed by tapping the eppendorf tubes of the samples 4-5 times with index finger and/or gently vortexing. Plasma samples can be use neat or diluted in the water. For example for 2× dilution (5 μL sample is mixed with 10 μL water).

Once appropriate dilution of samples is made, 5 μL of Blank, Standard and Urine/Plasma samples in duplicate is transferred into 96 well plate with the a clear U well. Due to the small volume of mouse plasma samples, plasma was not assayed in duplicates. Then, 200 μl of working reagent supplied in the kit is added to samples in the plate. Plate is covered with a 96 well cover or SaranWrap and incubation is continued at room temperature for 30 min by vigorous shaking After 30 min, the plate is read using a microplate reader at A₅₉₀ₙₘ.

### Summary

This uric acid assay is a quick and simple method that allows many samples to be assayed at once. The standard curve is consistently linear with a R²=˜0.99. Recently, it was observed that freeze/thawing of plasma and urine samples could cause lower reading of uric acid. Therefore, it was important to always prepare samples in the same way. To obtain reproducible meaningful data, all samples from each studies were collected during experiments, kept at >−20° C. but assayed at once at the end of the experiment.

Preliminary experiments show that a standard curve prepared with mice serum instead of water does not produce a linear curve. This experiment needs to be repeated using mice serum in the blank and not the blank provided.

### Example 13

### Purification of Recombinant Candida utilis Uricase

### Summary

This example describes a working purification procedure that can be used to obtain gram quantities of pure Candida utilis uricase enzyme for crystallization and subsequent preclinical studies.

### Introduction

Uricase or urate oxidase catalyzes the following overall reaction as the final step in purine degradation:

Uric acid+O₂+H₂O->5-hydroxyisourate+H₂O₂->allantoin+CO₂

Humans and many primates lack uricase enzyme activity due to a mutational event that occurred sometime in early primate evolution. Uric acid levels can accumulate due to increased purine metabolism or impaired excretion by the kidney. In addition a diet rich in purines can lead to elevated uric acid levels. Increased uric acid levels can result in the formation of urate stones in the kidneys and in some cases a type of arthritis called gout. An oral enzyme replacement therapy involving Uricase is being investigated as a way to decrease elevated plasma uric acid levels.

C. utilis uricase is a homotetrameric enzyme consisting of four 34 kDa monomers. The purification method described in this report was adapted from a method for recombinant C. utilis uricase purification described by Bomalaski et al, (2002) J. Rheumatol. 29:1942-9. This example provides the procedure used to purify gram quantities of C. utilis uricase expressed in E. coli for crystallization and animal studies.

### Equipment and Materials

### Reagents

Ammonium sulfate (Certified ACS Granular)—Fisher Scientific—CAS 7783-20-2
Sodium chloride—Fisher Scientific—CAS 7647-14-5
Sodium phosphate monobasic—Fisher Scientific—CAS10049-21-5
Sodium phosphate dibasic heptahydrate—Sigma—Cat No S9390-1 kg
Dithiothreitol (DTT)—Sigma—Cat No. D9779
Bio-Rad Protein Assay, Dye Reagent Concentrate—Bio-Rad—Cat No. 500-0006
BIO-SAFE™ Coomassie Blue G250 stain—Bio-Rad—Cat No. 161-0787
Q sepharose—GE Healthcare—Cat No. 17-0510-04
CHT Ceramic hydroxyapatite resin—Bio-Rad—Cat No. 157-0040
Boric acid—Sigma Aldrich CAS # 10043-35-3
Uric acid—Sigma, U-0881
NuPAGE 4-12% Bis-Tris Gel, Invitrogen, Cat No. NPO321
Seeblue Plus 2 Prestained Standard (4-250 kDa), Invitrogen, Cat No. LC5925
NuPAGE MES SDS Running Buffer (20×), Invitrogen, Cat No. NP0002-O₂

### Equipment

MICROFLUIDICS™, Model No: 1108; Serial No: 2004, 160
Beckman Coulter Avanti J-26X P
Sartorius Stadium biotech 0.45+0.2 um filters
Sartorius Stadium biotech 0.65 um filters
UV Spectrophotometer—Agilent 8453
Table top centrifuge, Beckman GS-6R
Microfuge, Baxter, Biofuge 13
Microtiter plate reader, Molecular Devices, Spectra max Plus
pH meter—Fisher Scientific—Accumet Excel XL60
Large stir plate—IKA C-MAG-HS7
Conductivity meter—OAKTON, Con 110 series
Kitchen Blender—Krups
TFF system—Millipore stainless steel membrane holder
Pump—Cole Palmer—Masterflex L/S
Ultrafiltration membrane—Millipore Biomax 30 kDa—Cat No. P2B030A01
Pellicon XL—Millipore 100 kDa—Cat No. PXB100C50
INDEX column
Cuvettes—UV compatible—Plastibrand—Cat No. 7591-70
Overhead stirrer—Heidolph—Type RZR1
Circulating water bath—Cole Palmer
SDS-PAGE gel running apparatus, Invitrogen

### Experimental Methods

### Buffer Preparation

50 mM Boric acid buffer: Dissolved 3.1 gm of boric acid in 950 mL of DI water. Adjusted pH to 8.0 with 4 N NaOH and made up the volume to 1 L. Checked pH again and adjusted to 8.0 if required.

0.5 M sodium phosphate, pH 7.5 and 0.5 M sodium phosphate, pH 8.5:

To prepare these buffers, first 1 L of 0.5 M sodium phosphate monobasic and 2 L 0.5 M sodium phosphate dibasic were prepared with DI water. After a three point calibration of the pH meter, the pH of the 0.5 M sodium phosphate dibasic solution was adjusted by adding the 0.5 M sodium phosphate monobasic solution until the desired pH (7.5 or 8.5) was reached. These 0.5 M sodium phosphate solutions were used to prepare Buffers A through F.

Buffer A—20 mM sodium phosphate, pH 7.5 (50 L): A 50 L beaker was filled with DI water almost to the 45 L mark. 2 L of the 0.5 M sodium phosphate, pH 7.5 was added and mixed well on a large stir plate. The final volume was adjusted to 50 L with DI water.

Buffer B—20 mM sodium phosphate, 50 mM NaCl, pH 7.5 (20 L): A large beaker was filled with DI water almost to the 15 L mark. 800 mL of the 0.5 M sodium phosphate, pH 7.5 and 58.44 g of NaCl were added and mixed well on a large stir plate. The final volume was adjusted to 20 L with DI water.

Buffer C—20 mM sodium phosphate, 1 M NaCl, pH 7.5 (20 L): A large beaker was filled with DI water almost to the 15 L mark. 800 mL of the 0.5 M sodium phosphate, pH 7.5 and 1168.8 g of NaCl were added and mixed well on a large stir plate. The final volume was adjusted to 20 L with DI water.

Buffer D—20 mM sodium phosphate, pH 8.5 (5 L): A 5 L beaker was filled with DI water almost to the 4.5 L mark. 200 mL of the 0.5 M sodium phosphate, pH 8.5 was added and mixed well on a stir plate. The initial volume was adjusted to 5 L with DI water.

Buffer E—50 mM sodium phosphate, pH 8.5 (5 L): A 5 L beaker was filled with DI water almost to the 4.5 L mark. 500 mL of the 0.5 M sodium phosphate, pH 8.5 was added and mixed well on a stir plate. The final volume was adjusted to 5 L with DI water.

Buffer F—100 mM sodium phosphate, pH 8.5 (5 L): A 5 L beaker was filled with DI water almost to the 4 L mark. 1 L of the 0.5 M sodium phosphate, pH 8.5 was added and mixed well on a stir plate. The final volume was adjusted to 5 L with DI water.

Buffer G—200 mM sodium phosphate, pH 8.5 (5 L): A 5 L beaker was filled with DI water almost to the 3 L mark. 2 L of the 0.5 M sodium phosphate, pH 8.5 was added and mixed well on a stir plate. The final volume was adjusted to 5 L with DI water.

Important to note: Fresh DTT was added to each buffer to a final concentration of 1 mM DTT just before use.

### Uricase Activity Assay

0.13 mM Uric acid (prepared fresh everyday): Dissolve ˜10 mg uric acid in an appropriate amount of 50 mM Boric acid buffer pH 8.0 to make it 1.3 mM (21.85 mg/100 mL). Dilute 1.3 mM uric acid 10 times to make it 0.13 mM using 50 mM Boric acid buffer pH 8.0.

### Assay Procedure

(i) A spectrophotometer is setup with the following parameters:
   Mode: Kinetics
   Absorbance: 292 nm
   Read time: 0-300 seconds at interval of 5 seconds
   Calculation for rate: Initial rate for 0-300 seconds
   Multiply by −60 to get AU/min
(ii) Turn on the circulating water bath for temperature control and set it at 37° C.
(iii) Take 3 mL of 50 mM Boric acid buffer pH 8.0 in a UV cuvette and blank it at 292 nm
(iv) Take 3 mL of 0.13 mM uric acid solution in cuvettes with tiny magnets for stirring.
(v) Take 10 uL of the sample and add to the 0.13 mM uric acid buffer in the cuvette and start measurement immediately.
(vi) Calculate the specific activity of the sample using the following equation:

$$Units \text{/} mg = \frac{rate \text{/} min * total reaction volume ( mL )}{12.3 * Sample volume ( mL ) * Sample conc . ( mg )}$$

Where12.3 is the molar extinction coefficient for uric acid.

E. coli Cell Lysis Using Microfluidizer

2 kg (2 bottle) of frozen cell paste (BL21(DE3)-pET9a-Uricase) were thawed at 4° C. overnight.

The frozen cell paste was resuspended in 10 L of Buffer A+1 mM DTT using a blender to process ˜250 g at a time.

Cells were lysed using the microfluidizer (with chamber pre-chilled on ice) by passing through two times at 90-100 psi. The lysate was kept on ice both before and after passing through the microfluidizer.

The crude lysate was centrifuged to pellet cell debris at 7800 RPM for 1 hour. (Note: if the Pellet is not Compact, it May be Necessary to Centrifuge for a Second Hour.)

The supernatant was collected into a large container, the volume was measured and it was assayed for uricase activity.

### 30% AMMONIUM SULFATE PRECIPITATION

Dry ammonium sulfate was added to the protein solution to reach 30% saturation (16.4 g ammonium sulfate per 100 mL). This solution was mixed using an overhead stirrer at 4° C. for a minimum of 3 hours.

The precipitate was pelleted by centrifugation at 7800 RPM for 1 hour.

The supernatant was collected into a large container and the volume was measured. Both the supernatant and the pellet were assayed for uricase activity. Uricase activity was detected only in the soluble fraction, so the pellet was discarded.

### 65% Ammonium Sulfate Precipitation

Additional ammonium sulfate was added to the protein solution to bring the final concentration to 65% saturation (21.4 g ammonium sulfate per 100 mL supernatant) and this mixture was stirred using an overhead stirrer for a minimum of 3 hours at 4° C.

The precipitate was pelleted by centrifugation at 7800 RPM for 1 hour.

Both the supernatant and the pellet were tested for uricase activity. The activity was only detected in the pellet fraction, so the supernatant was discarded.

The pellet was resuspended in Buffer A+1 mM DTT using an overhead stirrer for 1 hour at 4° C.

The suspension was centrifuged for 1 hour at 7800 RPM to pellet any insoluble material and then the supernatant was collected in a clean container.

### Filtration and Diafiltration

The protein solution was filtered at room temperature through 0.65 μm and 0.45 μm-0.2 μm filters in tandem. (Note: the 0.65 μm filter helped to keep the 0.45-0.2 μm filter from getting clogged.)

The protein solution was diafiltered into Buffer A+1 mM DTT using tangential flow filtration (Millipore membrane 100 kDa MW cut off) to reduce the salt concentration/conductivity to that of Buffer A. The permeate was continuously monitored for uricase activity.

After diafiltration the protein was stored at 4° C. before loading onto the Q sepharose column

### Anion Exchange Chromatography

At pH 7.5, Candida utilis uricase does not bind or binds very weakly to Q sepharose resin. This observation was published by Bomalaski et al.

A large Q sepharose column (2-3 L bed volume) was equilibrated with Buffer A+1 mM DTT at room temperature. The column was considered to be equilibrated when the pH and conductivity of the buffer going in and coming out of the column were identical.

The protein was loaded onto the column and the flow through was monitored for uricase activity. Once uricase started to elute from the column, the flow through was collected in a large container.

The column was washed with 5 CVs of Buffer A+1 mM DTT and this was collected with the flow through.

The column was then washed with 5 CVs of Buffer B+1 mM DTT and this material was collected in a separate large container. Once uricase activity was no longer detectable, the column was cleaned with Buffer C.

The column was disinfected with 0.1 N NaOH and stored at 4° C.

All of the fractions from the Q sepharose column were analyzed by SDS-PAGE (procedure according to Invitrogen's instructions for Bis-Tris gels) and by measurement of uricase activity.

Next, we proceeded to the hydroxyapatite column with the fractions containing significant uricase activity. The HA column (200 g resin) can accommodate about 10 g of total protein, so if there is significantly more material than that, multiple runs on the HA column are required. A larger version of this column is currently being tested.

Important to note: The HA resin needs to be washed, maintained and stored in phosphate buffer at a minimum concentration of 5 mM at near neutral pH so that the resin retains its binding activity.

### Hydroxyapatite Chromatography

The HA column (200 g of ceramic hydroxyapatite resin from Bio-Rad) was equilibrated with Buffer D+1 mM DTT before the uricase protein was loaded. The column was considered to be equilibrated when the pH and conductivity of the buffer going in and coming out of the column were identical.

If only the flow through fraction from the Q sepharose column has activity, the protein solution is concentrated to a manageable volume (10 g in ˜1-2 L) and then applied directly to the HA column. If however, the Buffer B wash also contains significant Uricase activity, this wash fraction is first concentrated and diafiltered with Buffer A+1 mM DTT to reduce the conductivity to that of Buffer A as measured by a conductivity meter before applying it to the HA column

The pH of the protein solution was adjusted to pH 8.5 before loading onto the HA column

The protein (˜10 g) was loaded onto the HA column at a flow rate of 30 mL/min and then washed with Buffer E+1 mM DTT while continuously monitoring for uricase activity.

The uricase enzyme was eluted with Buffer F+1 mM DTT and fractions were collected (50 mL fractions).

Uricase continued to be eluted with Buffer G+1 mM DTT and again fractions were collected (50 mL fractions).

The fractions were stored at 4° C. and analyzed by SDS-PAGE and uricase activity assays.

The fractions with high specific activity were pooled and concentrated using tangential flow filtration with a Millipore ultra filtration membrane (100 kDa MW cut off).

### Results

The four step procedure described above produces >95% pure C. utilis uricase as judged by SDS-PAGE with a specific activity of −40 U/mg. Table 2 shown below is a representative purification table for a typical uricase purification. Based on the results of this purification, the yield is ˜5 g uricase per 1 kg of frozen cell paste. After the two column purification the uricase can be crystallized.

TABLE 2
Example Uricase purification table

| | Volume | Protein | Total Protein | Total | Specific |
|---|---|---|---|---|---|
| Step | (mL) | (mg/mL) | (mg) | Activity | Activity |
| Cell Lysate | 10800 | 6.8 | 73440 | 167000 | 2.3 |
| AS Ppt | 6000 | 4.9 | 29400 | 114560 | 3.9 |
| TFF | 6000 | 4.5 | 27000 | 109999 | 4.1 |
| Anion Ex | 19500 | 1.3 | 25350 | 35999 | 1.4 |
| TFF | 3000 | 8.1 | 24300 | 67000 | 2.8 |
| Pre-load* | 900 | 11.6 | 10418 | 226066 | 21.7 |
| 50 mM | 500 | 1.8 | 900 | 30150 | 33.5 |
| NaPO4 | | | | | |
| 100 mM | 400 | 2.8 | 1106 | 57623 | 52.1 |
| NaPO4** | | | | | |
| 200 mM | 350 | 3.2 | 1137 | 40818 | 35.9 |
| NaPO4** | | | | | |
| Total yield^{†} | 1250 | 2.5 | 3143 | 128591 | 40.9 |

*The pre-load sample is ~⅓ of the total protein after the Q sepharose column.
**Values reported for the 100 mM NaPO4 and 200 mM NaPO4 elutions are a result of pooling fractions 6-13 for 100 mM and 6-12 for 200 mM.
^{†}Total yield refers to the combined 50 mM, 100 mM and 200 mM fractions.

### Conclusions

The procedure described here successfully produced uricase for crystallization and material that can be used for preclinical studies.

### Example 14

### Construction of Uricase Expression vectors and Evaluation of Recombinant Uricase Expression in E. coli

### Summary

This example describes the procedure used to generate both ampicillin and kanamycin resistant clones for the expression of Uricase enzymes from different organisms in E. coli. The generation of 6×His tagged Uricase constructs for easy purification and initial screening of the candidate enzymes will be described. This report will also cover the initial characterization of these enzymes on a small scale by SDS-PAGE analysis and activity assays.

### Introduction

Uricase or urate oxidase catalyzes the following overall reaction as the final step in purine degradation:

Uric acid+O₂+H₂O->5-hydroxyisourate+H₂O₂->allantoin+CO₂

Uricase can be found in many organisms from bacteria to mammals, however it is absent in humans and many primates due to a mutational event that occurred some time in early primate evolution. Although uric acid can play a positive role by scavenging free radicals, when too much uric acid is accumulated in the blood, hyperuricemia and gout can occur. Here the strategy is to reduce uric acid levels by oral delivery of uricase as an enzyme replacement therapy.

Oral delivery of an enzyme comes with a few challenges. First, we need an enzyme with high specific activity and stability over a wide pH range. For instance, the enzyme needs to withstand the low pH in the gastric compartment in order to provide its function in the intestinal tract where the pH is closer to neutral. In addition, the enzyme has to be resistant to proteases that reside within the GI tract. Some of these stability issues can be addressed at the formulation level, and/or proper enzyme selection can aid downstream steps.

This example describes the method used for the selection of uricase enzyme for oral delivery. First, candidate selection will be discussed followed by gene optimization and synthesis. Next the subcloning of the uricase genes into appropriate expression vectors will be described. Here, multiple constructs were generated for each clone because different enzymes could be expressed differently and could behave differently depending on the antibiotic used. Previous research on other projects suggested that kanamycin resistance, although preferred for large scale protein purification under cGMP regulations, often leads to lower protein expression levels than ampicillin resistant clones. Due to this, both ampicillin and kanamycin clones were generated for comparison.

### Equipment and Materials

### Reagents

NEB 5-alpha F′I^{q} Competent E. coli, New England Biolabs, Cat No. C2992
NdeI, New England Biolabs, Cat No. R0111S
BamHI, New England Biolabs, Cat No. R0136S
Wizard DNA miniprep kit, Promega, Cat No. PR-A1460
Wizard DNA PCR purification kit, Promega, Cat No. PR-A7170
Ampicillin, Sigma, Cat No. A9518
Kanamycin, Gibco, Cat No. 11815-024
LB, MP Biomedicals, Cat No. 3002-131
LB-Agar, MP Biomedicals, Cat No. 3002-331
BugBuster Protein Extraction Reagent: Novagen, Cat. No. 70584-4
BL21(DE3) E. coli cells: Stratagene, Cat No. 50125011
IPTG, Sigma, Cat No. 15502
pET11a, Novagen, Cat No. 69436-3
pET9a, Novagen, Cat No. 69431-3
1 kb DNA ladder, New England Biolabs, Cat No. NO468S
10×DNA loading Buffer, Invitrogen, Cat No. 10816-015
Quick ligation kit, New England Biolabs, Cat No. M2200L
Antarctic phosphatase, New England Biolabs, Cat No. M0289L
SOC medium, New England Biolabs, Cat No. B9020S
BIO-SAFE™ Coomassie Blue G250 stain, Bio-Rad, Cat No. 161-0787
NuPAGE 4-12% Bis-Tris Gel, Invitrogen, Cat No. NPO321
Seeblue Plus 2 Prestained Standard (4-250 kDa), Invitrogen, Cat No. LC5925
NuPAGE MES SDS Running Buffer (20×), Invitrogen, Cat No. NP0002-02
URIC1_SOYBN Geneart #0709650
URIC_PHAVU Geneart #0709651
URIC_ARTGL Geneart #0709652
URIC_PICJA Geneart #0709653
URIC_PSEAE Geneart #0709654

### Equipment

Thermocycler, Eppendorf
Table top centrifuge, Beckman GS-6R
Microfuge, Baxter, Biofuge 13
UV-Spectrophotometer, Agilent 8453
Microtiter plate reader, Molecular Devices, Spectra max Plus
Sybr Green, Invitrogen, S-7563
Thin walled PCR tubes, Brinkmann Instruments, Cat No. E0030 124 260
Circulating water bath, Cole Palmer
SDS-PAGE gel running apparatus, Invitrogen

### Experimental Methods

### Gene Selection

A literature search and a search of the BRENDA enzyme database were used to identify the best candidate uricase enzymes for oral delivery. Key features that were compared were enzyme stability at different pHs, pH range for enzyme activity, thermal stability and specific activity.

### Generating Clones for Candidate Uricases

Gene sequences for selected uricases (Glycine max, Phaseolus vulgaris, Candida utilis, Arthrobacter globiformis, Pseudomonas aeruginosa) were obtained from GenBank. The sequences from plant, yeast and bacteria were then submitted to Geneart AG for sequence optimization for E. coli expression followed by gene synthesis. Geneart AG delivered plasmids (10 ug of each plasmid in lyophilized form) containing the sequence optimized uricase gene of interest with documentation for full sequencing of the gene. The plasmids were resuspended in 50 uL of sterile water and stored at −20° C.

Subcloning of the Uricase Genes into Expression Vectors for E. coli

### Subcloning Strategy

Expression vectors pET9a (kanamycin resistance) and pET11a (ampicillin resistance) were selected for Uricase protein expression in E. coli (For plasmid maps, please see Appendices). The multiple cloning sites in both vectors have NdeI and BamHI restriction sites (sites not present in the uricase genes). Oligonucleotides were designed for the amplification of the synthetic uricase genes to add Nde I and BamHI sites to the 5′ and 3′ ends of the gene respectively (Table 3). Two forward primers for each gene were synthesized, one to add an N-terminal 6×His tag and the other to generate untagged uricase.

TABLE 3
Oligonucleotides used for Uricase gene
amplification

| PRIMER NAME | PRIMER SEQUENCE |
|---|---|
| 5URICSB | 5′ AGATATACATATGGCGCAGCAGGAAGTGGTTGAAG |
| | GC 3′ |
| 5HisURICSB | 5′ AGATATACATATGCACCATCACCATCACCATGCGC |
| | AGCAGGAAGTGGTTGAAGGC 3′ |
| 3URICSB | 5′ TATGGATCCTTACAGTTTGCTCCACAGACGG 3′ |
| 5URICAG | 5′ AGATATACATATGACCGCGACCGCGGAAACCAGC 3′ |
| 5HisURICAG | 5′ AGATATACATATGCATCACCATCACCATCACACCG |
| | CGACCGCGGAAACCAGC 3′ |
| 3URICAG | 5′ AATGGATCCTTAGCAAAAGCCCGCAATGTTGC 3′ |
| 5URICPV | 5′ AGATATACATATGGCGCAGGAAGTGGTTGAAGGC 3′ |
| 5HisURICPV | 5′ AGATATACATATGCATCACCATCACCATCACGCGC |
| | AGGAAGTGGTTGAAGGC 3′ |
| 3URICPV | 5′ ATTGGATCCTTACAGTTTGCTCCACACACGG 3′ |
| 5URICPJ | 5′ AGATATACATATGAGCACCACCCTGAGCAGCAGC 3′ |
| 5HisURICPJ | 5′ AGATATACATATGCATCACCATCACCATCACAGCA |
| | CCACCCTGAGCAGCAGC 3′ |
| 3URICPJ | 5′ ATAGGATCCTTACAGTTTGGTTTTTTCTTTACGCA |
| | CC 3′ |
| 5URICPA | 5′ AGATATACATATGCCGAAAAGCAGCGCGGCG 3′ |
| 5HisURICPA | 5′ AGATATACATATGCATCACCATCACCATCACCCGA |
| | AAAGCAGCGCGGCGGAAC 3′ |
| 3URICPA | 5′ TAAGGATCCTTACGCCGCAACCGGCGGCGGC 3′ |

Note: Two letter abbreviations for each of the candidate uricases: Glycine max: SB; Arthrobacter globiformis: AG; Phaseolus vulgaris: PV; Candida utilis: PJ; Pseudomonas aeruginosa: PA.

### PCR Reaction Mix

Oligonucleotides were received as lyophilized powders which were resuspended in sterile water to a final concentration of 50 uM. Invitrogen's Platinum Pfx DNA polymerase was used for PCR amplification following the manufacturer's instructions. A master mix was prepared as follows: 120 uL of Platinum Pfx Amplification Buffer, 36 uL of 10 mM dNTPs, 24 uL 50 mM MgSO₄, and 969.6 uL sterile water were mixed thoroughly. The master mix was thoroughly mixed and 95.8 uL was distributed into each thin walled PCR tube. 2 uL of 0.025 ug/uL template DNA, 0.6 uL of each primer (50 uM stocks), and 1 uL of Platinum Pfx DNA polymerase were then added to each tube.

### Thermocycler Conditions for PCR

Initial denaturation was at 94° C. for 2 min, followed by 30 cycles of denaturation for 15 s at 94° C., annealing at 55° C. for 30 s and extension for 2 min at 68° C.

Reaction products were analyzed for size and quality by running 5 uL of each 100 uL reaction on a 1% agarose gel.

### Restriction Enzyme Digests

PCR products were purified using the Wizard DNA purification kit from Promega and eluted with 50 uL sterile water. pET9a and pET11a were amplified in NEB 5-alpha cells and purified using the Wizard plasmid miniprep kit. DNA concentrations were determined by measuring the absorbance of the DNA at 260 nm (where OD₂₆₀ₙₘ=1 is 50 ug/mL dsDNA. Both the pET vectors and the uricase PCR products were digested first with NdeI as follows in 30 uL reactions: 3 uL 10× Buffer 4 (NEB), ˜1 ug of DNA, and 2 uL NdeI (NEB) and sterile water to bring volume to 30 uL final. This reaction was incubated overnight at 37° C. The following morning the salt concentration was increased to 350 uM using 5 M NaCl and then 1 uL of BamHI was added to each tube (for the pET vectors 2 uL of BamHI was used). These reactions were incubated at 37° C. for 3 hours at which point the restriction enzymes were heat inactivated at 80° C. for 20 min.

### Ethanol Precipitation of DNA

75 uL of cold 95% ethanol was added to each restriction digest and mixed well. This mixture was incubated for 20 min on ice then centrifuged at maximum speed for 15 min to pellet the precipitated DNA. The ethanol was aspirated and then the DNA pellet was washed with 70% ethanol and centrifuged again. The DNA pellet was allowed to air dry for a few minutes and then it was resuspended in 20 uL of sterile water. DNA concentrations were again determined by measuring the OD₂₆₀ₙₘ.

### Phosphatase Treatment of Digested Vectors

The pET9a and pET11a vectors were treated with Antarctic phosphatase to remove the 5′ phosphates. The reactions were set up as follows: 17 uL of digested pET vector DNA, 2 uL 10× Antarctic phosphatase buffer and 2 uL Antarctic phosphatase enzyme. The reactions were incubated at 37° C. for 1 hour. The Antarctic phophatase enzyme was finally heat inactivated by incubation at 65° C. for 5 min.

### Dna Ligations

Ligation reactions were set up using reagents from the Quick ligation kit purchased from New England Biolabs. The following formula was used to determine how much vector and insert were used in the ligation reactions:

$$\frac{ng of vector DNA \times size of insert DNA}{size of plasmid DNA} = ng of insert DNA for a 1 \text{:} 1 molar ratio$$

For example:

$$\frac{50 ng pET 9 a \times 1000 bp ( ∼ size of uricase gene )}{4300 bp ( size of pET 9 a )} = \begin{matrix}12 ng insert for 1 \text{:} 1 ratio \\ 108 ng for 1 \text{:} 9 ratio\end{matrix}$$

A 1:9 molar ratio between vector and insert was used for all ligation reactions. Reaction mixtures included 50 ng digested and phosphatase treated vector, 108 ng uricase insert DNA, 10 uL 2× Ligase Buffer, and 1 uL DNA ligase. A no insert control (just vector, buffer and enzyme) reaction was set up for each vector. Reactions were incubated for 5 min at room temperature.

2.5 uL of each ligation reaction was used to transform 50 uL NEB 5-alpha competent E. coli cells (NEB) according to the manufacturer's instructions. Following a 1 hour outgrowth, 200 uL of the cells were plated onto selective agar plates, either with kanamycin or ampicillin. Plates were incubated at 37° C. overnight for 15-18 hrs.

### Screening and Clone Confirmation

5 mL cultures were grown for transformants for screening for the ligation products. Plasmid DNA was purified using the Wizard miniprep kit (Promega). To screen for positive clones, the plasmid DNA was digested with Pst I. There is a single site for Pst I within the Uricase gene sequences. For pET9a, there are no Pst I sites within the vector, so pET9a candidates that are linearized by Pst I should contain the uricase gene insert. For pET11a, there is a single Pst I site in the vector sequence. Pst I digestion of pET11a candidates should excise a DNA fragment of ˜1500 bp if the uricase insert is present. Positive clones identified by Pst I digest were sent for DNA sequencing (Agencourt) using both the Universal T7 (5′ TAA-TAC-GAC-TCA-CTA-TAG-GG 3′) and T7 terminator (5′ CTA-GTT-ATT-GCT-CAG-CGG 3′) primers.

Transformation into the BL21(DE3) E. coli Strain

Uricase expression vectors were transformed into competent BL21(DE3) E. coli cells (Stratagene) using the manufacturers recommended chemical transformation procedure. Cells were grown overnight on LB/Agar plates with the appropriate antibiotic at 37° C. Single colonies were selected for evaluation of protein expression and uricase enzyme activity.

### Small Scale Expression of Uricases

Small scale expression was performed to find the best clone for high level expression of the soluble enzyme. Small overnight cultures 1-3 mL were inoculated with single colonies (BL21(DE3) containing uricase expression vectors) and grown at 37° C. with shaking at 250 rpm. These starter cultures were used to inoculate larger cultures of 20-50 mL for protein expression. Cell growth was monitored by measurements of optical density at 595 nm. At OD₅₉₅ₙₘ between 0.5 and 0.9, protein expression was induced with 1 mM IPTG for 3 hrs. at 37° C. 1.5 mL samples were taken before and after induction. The same number of cells was harvested for each of the cultures so the expression levels could be adequately compared. The cells were pelleted and frozen at −20° C. for analysis by SDS-PAGE and uricase activity assays (see below).

### Protein Extraction and Analysis

Cell pellets were thawed on ice and cells were lysed using BugBuster reagent from Novagen. 300 uL BugBuster reagent with 1 mg/mL lysozyme and Benzonase was added to each cell pellet and then incubated on a rotator at room temperature for 20 min to lyse the cells. The crude lysate was centrifuged for 10 min at maximum speed to pellet cell debris and insoluble protein. The supernatant was then transferred to a fresh tube and placed on ice. The pellet was resuspended in another 300 uL of Bugbuster reagent so that the insoluble material could be analyzed as well. Both the supernatant and the pellet were analyzed by SDS-PAGE following Invitrogen's instructions and activity assays.

### Uricase Activity Assay

0.13 mM Uric acid (prepared fresh everyday): Dissolve ˜10 mg uric acid in an appropriate amount of 50 mM Boric acid buffer pH 8.0 to make it 1.3 mM (21.85 mg/100 mL). Dilute 1.3 mM uric acid 10 times to make it 0.13 mM using 50 mM Boric acid buffer pH 8.0.

### Assay Procedure

(vii) A spectrophotometer is setup with the following parameters:
   Mode: Kinetics
   Absorbance: 292 nm
   Read time: 0-300 seconds at interval of 5 seconds
   Calculation for rate: Initial rate for 0-300 seconds
   Multiply by −60 to get AU/min
(viii) Turn on the circulating water bath for temperature control and set it at 37° C.
(ix) Take 3 mL of 50 mM Boric acid buffer pH 8.0 in a UV cuvette and blank it at 292 nm
(x) Take 3 mL of 0.13 mM uric acid solution in cuvettes with tiny magnets for stirring.
(xi) Take 10 uL of the sample and add to the 0.13 mM uric acid buffer in the cuvette and start measurement immediately.
(xii) Calculate the specific activity of the sample using the following equation:

$$Units \text{/} mg = \frac{rate \text{/} min * total reaction volume ( mL )}{12.3 * Sample volume ( mL ) * Sample conc . ( mg )}$$

Where12.3 is the molar extinction coefficient for uric acid.

### Results

### Uricase Gene Selection and Sequences

Extensive literature searches as well as searches of the BRENDA enzyme database revealed Uricases with specific activities up to 30 U/mg with one striking exception of 636 U/mg for P. aeruginosa. Table 4 shows the available information for the uricases that were selected for further evaluation. Five different Uricases from plant, yeast and bacterial sources were selected for further evaluation in house. The Uricases selected were from Candida utilis, Pseudomonas aeruginosa, Glycine max, Phaseolus vulgaris, and Arthrobacter globiformis. These enzymes were selected to get a sampling of different uricase enzymes from different species. These enzymes have pH optima ranging from 7-9.5. In addition to characteristics suitable for oral delivery, we would like to crystallize these enzymes and it is difficult to predict whether or not a protein will crystallize. Thus, by sampling a diverse array of enzymes, we can identify enzymes that are amenable to crystallization which may facilitate formulation for oral delivery.

TABLE 4
Characteristics of uricases chosen for further evaluation in house

| | CANDIDA | ARTHROBACTER | PSEUDOMONAS | | PHASEOLUS |
|---|---|---|---|---|---|
| | UTILIS | GLOBIFORMIS | AERUGINOSA | GLYCINE MAX | VULGARIS |
| Specific | 20-30 | 30 | 636* | 3.6 | 1.8 |
| activity | | | | | |
| (u/mg) | | | | | |
| pH | 7-8 | 8-9 | 9 | 9.5 | 9-9.5 |
| optimum | | | | | |
| Thermal | 30-35 | | 30 | 30-35 | |
| stability | | | | | |
| pI | 8.5 | ~5 | 5.5 | 7.7 | 8.3 |
| Tertiary | Tetramer | Tetramer | Monomer | Tetramer | Tetramer |
| structure | (4 × 34 kDa) | (4 × 33 kDa) | (64 kDa) | (4 × 35 kDa) | (4 × 34 kDa) |

*The specific activity of the P. aeruginosa enzyme is reported by Saeed et al. (2004) Polish Journal of Microbiology, 53: 45-52. The enzyme tested in their study is the native enzyme secreted from P. aeruginosa. They compare the P. aeruginosa enzyme with C. utilis and observe that over time more uric acid is degraded by the P. aeruginosa uricase, however the initial velocities appear to be identical. Thus, the activity of the enzyme will need to be confirmed in house.

The following are the sequences for the various uricases obtained from Genbank

Glycine max (Soybean) uricase: “URIC1_SOYBN”
ATGGCTCAGCAGGAAGTGGTAGAAGGGTTCAAGTTCGAACAGAGGCACGG
GAAAGAACGCGTGAGAGTAGCGCGCGTGTGGAAGACGAGGCAGGGGCAGC
ACTTCATTGTGGAGTGGCGCGTGGGGATCACTCTCTTTTCGGATTGCGTC
AACTCGTACCTCCGCGATGACAACTCTGAAATCGTTGCTACTGATACCAT
GGAAAAACACCGTGTATGCAAAACAAAGGAATGCTCTGACATACTTTCTG
CCGAGGAGTTTGCTATTCTGCTTGCTAAGCACTTTGTATCATTTTACCAG
AAGGTTACTGGTGCTATTGTGAATATTGTGGAAAAACCATGGGAGCGTGT
CACTGTGGATGGTCAACCTCATGAACATGGTTTCAAACTTGGGTCTGAGA
AGCATACAACAGAGGCGATAGTACAAAAGTCTGGTTCACTTCAGTTGACT
TCTGGTATTGAAGGATTGTCAGTGTTGAAGACAACCCAGTCTGGTTTTGT
GAATTTCATAAGAGACAAGTACACAGCACTTCCTGATACCCGTGAAAGGA
TGGTAGCAACAGAAGTAACCGCACTGTGGAGGTATTCGTATGAATCGCTG
TATAGCCTCCCTCAGAAGCCGCTTTACTTTACAGAAAAGTATCAGGAAGT
GAAAAAAGTTCTGGCTGACACTTTTTTTGGCCCACCAAAAGGGGGAGTCT
ATAGCCCATCTGTTCAAAACACACTCTACCTGATGGCAAAGGCCACACTG
AACAGATTTCCTGACATAGCTTATGTCAGTCTAAAGTTGCCAAATCTTCA
TTTCATACCTGTCAATATCTCAAACCAGGATGGCCCTATTGTGAAGTTTG
AGGATGATGTGTACTTGCCAACGGATGAGCCACATGGGTCAATTCAAGCT
AGTTTGAGCCGCCTTTGGTCAAAGCTGTAG
Phaseolus vulgaris uricase: “URIC_PHAVU”
ATGGCGCAGGAAGTTGTGGAGGGGTTCAAGTTTGAGCAGAGGCACGGGAA
AGAGCGCGTCAGAGTTGCGCGCGTGTGGAGGACGCCGCAGGGTCGCCACT
TCGTTGTGGAGTGGCGCGTAGGTATTACTCTCTTCTCTGATTGCGTCAAC
TCGTATCTCCGCGATGATAACTCTGACATCGTTGCCACTGACACCATGAA
AAACACGGTGTATGCAAAAGCAAAGGAATGCTCGGATATACTTTCTGTCG
AGGACTTTGCTATTCTACTTGCCAAGCACTTTGTATCATTTTACAAGAAG
GTTACTGGTGCTATTGTGAATATCGTGGAAAAACCATGGGAGCGTGTCAT
TGTGGATGGTCAACCTCATCAACATGGTTTCACACTTGGGTCTGAGAAGC
ATACAACAGAGGCAATAGTACAGAAGTCTGGTTCACTACAATTGACTTCT
GGTATTGAAGGATTGTCAGTGTTGAAGACAACCCAGTCTGGTTTTGAGAA
TTTCATTAGAAACAAGTACACAGCACTTCCAGATACCCGCGAAAGGATTT
TGGCAACAGAAGTAACTGCTCTGTGGAGGTATTCGTACGAATCTCTATAC
AACCTCCCTCAGAAGCCACTATACTTCACAGACAAGTATCTGGAAGTGAA
AAAAGTTCTGGCTGACACATTTTTTGGGCCACCAAACAGGGGAGTCTATA
GCCCATCTGTTCAAAACACACTCTACCTTATGGCAAAGGCCACACTGAAC
AGATTTCCTGACATTGCTTATGTCCATCTAAAGATGCCAAATCTTCATTT
CTTACCAGTCAACATCTCAAGCAAGGATGGTCCAATTGTGAAGTTTGAGG
ATGATGTTTATTTACCAACGGACGAGCCTCATGGCTCAATTGAAGCAAGC
TTGAGCCGGGTTTGGTCAAAGCTGTAG
Arthrobacter globiformis uricase: “URIC_ARTGL”
ATGACTGCCACCGCAGAAACCTCAACCGGCACCAAGGTCGTGCTCGGACA
GAACCAGTACGGCAAGGCCGAAGTCCGCCTCGTCAAGGTCACGCGCAATA
CCGCCCGGCACGAGATCCAGGACCTGAATGTCACCTCGCAGCTGCGCGGC
GACTTCGAGGCCGCACACACCGCCGGCGACAACGCGCACGTGGTCGCCAC
CGACACGCAGAAGAACACCGTCTACGCCTTCGCCCGCGACGGCTTCGCCA
CCACCGAGGAGTTCCTGCTCCGGCTGGGCAAACACTTCACCGAGGGCTTC
GACTGGGTAACCGGCGGGCGCTGGGCGGCGCAGCAGTTCTTCTGGGACCG
CATCAACGACCACGACCACGCCTTCTCCCGGAACAAGAGCGAGGTCCGCA
CCGCCGTGCTCGAGATCTCGGGCAGCGAGCAGGCCATCGTCGCCGGGATC
GAGGGCCTGACGGTCCTGAAGTCCACCGGTTCGGAATTCCACGGCTTCCC
GCGGGACAAGTACACCACCCTGCAGGAAACCACCGACCGTATCCTCGCCA
CGGATGTCAGCGCCCGCTGGCGCTACAACACCGTCGAGGTTGACTTCGAC
GCCGTCTACGCGAGCGTCCGCGGGCTGCTGCTCAAGGCCTTCGCCGAGAC
CCACTCGCTGGCCCTGCAGCAGACCATGTATGAGATGGGCCGGGCCGTCA
TCGAGACGCACCCGGAAATCGACGAAATCAAGATGTCCCTGCCGAACAAG
CACCATTTCCTGGTGGACCTGCAGCCCTTCGGACAGGACAACCCGAATGA
GGTGTTCTACGCCGCCGACCGTCCCTACGGACTGATCGAAGCCACCATCC
AGCGCGAGGGCTCGCGCGCCGACCACCCGATCTGGTCGAACATCGCCGGA
TTCTGCTAG
Pichia jadinii (Candida utilis) uricase:
“URIC_PICJA”
ATGTCAACAACGCTCTCATCATCCACCTACGGCAAGGACAACGTCAAGTT
CCTCAAGGTCAAGAAGGACCCGCAAAACCCAAAGAAGCAGGAGGTTATGG
AGGCCACCGTCACGTGTCTGCTTGAAGGTGGGTTCGACACCTCGTACACG
GAGGCTGACAACTCGTCCATCGTGCCAACAGACACCGTGAAGAACACCAT
TCTCGTGTTGGCAAAGACCACGGAGATTTGGCCAATTGAGAGATTTGCAG
CCAAGCTGGCCACGCACTTTGTTGAGAAGTACTCGCACGTCTCTGGCGTC
TCCGTCAAGATTGTCCAGGACAGATGGGTCAAGTACGCCGTTGATGGCAA
GCCACACGACCACTCTTTTATCCACGAAGGTGGTGAGAAGAGAATCACTG
ACCTGTACTACAAGAGATCCGGTGATTACAAGCTGTCGTCTGCCATCAAG
GACTTGACGGTGCTGAAGTCCACCGGCTCGATGTTCTACGGCTACAACAA
GTGTGACTTCACCACCTTGCAACCAACAACTGACAGAATCTTGTCCACCG
ACGTCGATGCCACCTGGGTTTGGGATAACAAGAAGATTGGCTCTGTCTAC
GACATCGCCAAGGCTGCAGACAAGGGAATCTTTGACAACGTTTACAACCA
GGCTAGAGAGATCACCTTGACCACCTTTGCTCTCGAGAACTCTCCATCTG
TGCAGGCCACGATGTTCAACATGGCTACTCAGATCTTGGAAAAGGCATGC
TCTGTCTACTCGGTTTCATACGCCTTGCCAAACAAGCACTACTTCCTCAT
TGACTTGAAATGGAAAGGTTTGGAGAACGACAACGAGTTGTTCTACCCAT
CTCCACATCCAAATGGGTTGATCAAGTGTACTGTTGTCCGTAAGGAGAAG
ACCAAGTTGTAG
Pseudomonas aeruginosa uricase: “URIC_PSEAE”
ATGCCCAAGTCATCCGCCGCCGAACAATCCGGCGAGAGTTCGACCCAGAC
CCTGTCCCTGCTCGACGAGATCATCGCCAAGGGCCGCATGGCCCACGACG
ACAGCCAGCAGGACTATGCCCGCGACATGCTCGCGGAATTCGCCACCCAG
GTCCTCGACGAGGGCATGGCCGTCGACAAGGACACCGTGGCGATGATCAA
CGACCGCATCAGCCAGATCGATGCGCTGATCAGCGACCAGCTCAACCAGA
TCATCCACCACCCCGAGTTGCAGAAGCTGGAAGCCTCCTGGCGCGGCCTG
CACCAACTGGTGAGCAACACCGAGACCAGCGCGCGGCTCAAGCTGCGCCT
GCTGAACGTCGGCAAGAACGAACTGCAGAACGACCTGGAGAAGGCGGTCG
AGTTCGACCAGAGCGCACTGTTCAAGAAGATCTACGAAGAGGAATACGGC
ACCTTCGGCGGACAGCCCTTCAGCCTGCTGATCGGCGACTTCACCTTCGG
TCGCCATCCGCAGGACATCGGCCTGCTGGAGAAGCTGTCGAACGTCGCCG
CGGCTGCCCACGCGCCGTTCATCGCCGCCGCCAGCCCACGCCTGTTCGAC
ATGAACAGCTTCACCGAACTGGCCGTGCCGCGCGACCTGACCAAGATCTT
CGAGAGCCTGGAGCTGATCAAGTGGCGCGCCTTCCGCGAGAGCGAGGACT
CGCGCTACGTGTCGCTGGTGCTGCCGAACTTCCTCCTGCGCCTGCCCTAC
GGCCCGGAGACGCGCCCGGTGGAAGGCATGAACTATGTCGAGGACGTCAA
CGGCACCGACCACTCCAAGTACCTCTGGGGCAACGCCGCCTGGGTCCTGG
CGCAGCGCATCACCGAGGCCTTCGCCAAGTACGGCTGGTGCGCGGCGATC
CGCGGCGCGGAAGGCGGCGGCGCGGTCGAGGGCCTGCCGGCGCACACGTT
CCGCACCAGCTCCGGCGACCTGTCGCTGAAGTGCCCGACCGAGGTGGCGA
TCACCGACCGCCGCGAGAAGGAACTCAACGATCTCGGCTTCATTTCCCTG
TGCCACAAGAAGAACAGCGACGTGGCGGTGTTCTTCGGCGGCCAGACCAC
CAACAAGGCCAGGCTCTACAACACCAACGAGGCCAACGCCAACGCGCGCC
TGTCGGCGATGCTGCCGTACGTGCTGGCGGCATCGCGCTTCGCCCACTAC
CTGAAGGTGATCATGCGCGACAAGGTCGGCAGCTTCATGACCCGCGACAA
CGTGCAGACCTACCTGAACAACTGGATCGCCGACTACGTGCTGATCAACG
ACAACGCACCGCAGGAAATCAAGGCGCAGTACCCGCTGCGCGAGGCGCGG
GTGGATGTCAGCGAGGTGGCCGGCAAACCGGGGGCCTACCGCGCCACGGT
GTTCCTCCGGCCGCACTTCCAGCTCGAGGAACTCAGCGCGTCGATCCGCC
TGGTCGCCAACCTGCCGCCGCCGGTAGCGGCGTGA

### Sequence Optimized Uricase Genes

The above uricase gene sequences were optimized for expression in E. coli and synthesized by Geneart AG. Geneart provided each of the sequence verified clones in one of their standard plasmids (not an expression vector). The following are the optimized sequences of the uricase genes from Geneart.

URIC1_SOYBN (Geneart # 0709650):
ATGGCGCAGCAGGAAGTGGTTGAAGGCTTTAAATTTGAACAGCGCCATGG
CAAAGAACGTGTGCGTGTGGCGCGTGTGTGGAAAACCCGTCAGGGCCAGC
ATTTTATTGTGGAATGGCGTGTTGGCATTACCCTGTTTAGCGATTGCGTG
AACAGCTATCTGCGTGATGATAACAGCGAAATTGTGGCGACCGATACCAT
GAAAAACACCGTGTATGCGAAAGCGAAAGAATGCAGCGATATTCTGAGCG
CGGAAGAATTTGCGATTCTGCTGGCCAAACATTTTGTGAGCTTCTACCAG
AAAGTGACCGGCGCGATTGTGAACATTGTGGAAAAACCGTGGGAACGTGT
GACCGTGGATGGCCAGCCGCATGAACATGGCTTTAAACTGGGCAGCGAAA
AACATACCACCGAAGCGATTGTGCAGAAAAGCGGCAGCCTGCAGCTGACC
AGCGGCATTGAAGGCCTGAGCGTGCTGAAAACCACCCAGAGCGGCTTTGT
GAACTTTATCCGCGATAAATATACCGCGCTGCCGGATACCCGCGAACGTA
TGGTGGCGACCGAAGTGACCGCGCTGTGGCGTTATAGCTATGAAAGCCTG
TATAGCCTGCCGCAGAAACCGCTGTATTTCACCGAAAAATATCAGGAAGT
GAAAAAAGTTCTGGCCGATACCTTTTTTGGTCCGCCGAAAGGCGGCGTGT
ATAGCCCGAGCGTGCAGAACACCCTGTATCTGATGGCGAAAGCGACCCTG
AACCGTTTTCCGGATATTGCGTATGTGAGCCTGAAACTGCCGAACCTGCA
TTTTATTCCGGTGAACATCAGCAACCAGGATGGCCCGATTGTGAAATTTG
AAGATGATGTGTATCTGCCGACCGATGAACCGCATGGCAGCATTCAGGCG
AGCCTGAGCCGTCTGTGGAGCAAACTGTAA
URIC_PHAVU (Geneart #: 0709651)
ATGGCGCAGGAAGTGGTTGAAGGCTTTAAATTTGAACAGCGCCATGGCAA
AGAACGTGTGCGTGTGGCGCGTGTTTGGCGTACCCCGCAGGGCCGTCATT
TTGTGGTGGAATGGCGTGTTGGCATTACCCTGTTTAGCGATTGCGTGAAC
AGCTATCTGCGTGATGATAACAGCGATATTGTGGCGACCGATACCATGAA
AAACACCGTGTATGCGAAAGCGAAAGAATGCAGCGATATTCTGAGCGTGG
AAGATTTTGCGATTCTGCTGGCCAAACATTTTGTGAGCTTCTATAAAAAA
GTGACCGGCGCGATTGTGAACATTGTGGAAAAACCGTGGGAACGTGTGAT
TGTGGATGGCCAGCCGCATCAGCATGGCTTTACCCTGGGCAGCGAAAAAC
ATACCACCGAAGCGATTGTGCAGAAAAGCGGCAGCCTGCAGCTGACCAGC
GGCATTGAAGGCCTGAGCGTGCTGAAAACCACCCAGAGCGGCTTTGAAAA
CTTTATCCGCAACAAATATACCGCGCTGCCGGATACCCGCGAACGTATTC
TGGCCACCGAAGTGACCGCGCTGTGGCGTTATAGCTATGAAAGCCTGTAT
AACCTGCCGCAGAAACCGCTGTATTTCACCGATAAATATCTGGAAGTGAA
AAAAGTGCTGGCCGATACCTTTTTTGGTCCGCCGAACCGTGGCGTGTATA
GCCCGAGCGTGCAGAACACCCTGTATCTGATGGCGAAAGCGACCCTGAAC
CGTTTTCCGGATATTGCGTATGTGCATCTGAAAATGCCGAACCTGCATTT
TCTGCCGGTGAACATTAGCAGCAAAGATGGCCCGATTGTGAAATTTGAAG
ATGATGTGTATCTGCCGACCGATGAACCGCATGGCAGCATTGAAGCGAGC
CTGAGCCGTGTGTGGAGCAAACTGTAA
URIC_ARTGL (Geneart # 0709652):
ATGACCGCGACCGCGGAAACCAGCACCGGCACCAAAGTGGTGCTGGGCCA
GAACCAGTATGGCAAAGCGGAAGTGCGTCTGGTGAAAGTGACCCGTAACA
CCGCGCGTCATGAAATTCAGGATCTGAACGTGACCAGCCAGCTGCGTGGC
GATTTTGAAGCGGCGCATACCGCGGGTGATAACGCGCATGTGGTGGCGAC
CGATACCCAGAAAAACACCGTGTATGCGTTTGCGCGTGATGGCTTTGCGA
CCACCGAAGAATTTCTGCTGCGTCTGGGCAAACACTTTACCGAAGGCTTT
GATTGGGTGACCGGCGGTCGTTGGGCGGCGCAGCAGTTTTTTTGGGATCG
CATCAACGATCATGATCATGCGTTTAGCCGTAACAAAAGCGAAGTGCGTA
CCGCGGTGCTGGAAATTAGCGGCAGCGAACAGGCGATTGTGGCGGGCATT
GAAGGCCTGACCGTGCTGAAAAGCACCGGCAGCGAATTTCATGGCTTTCC
GCGCGATAAATATACCACCCTGCAGGAAACCACCGATCGTATTCTGGCCA
CCGATGTGAGCGCGCGTTGGCGTTATAACACCGTGGAAGTGGATTTTGAT
GCGGTGTATGCGAGCGTGCGTGGCCTGCTGCTGAAAGCGTTTGCGGAAAC
CCATAGCCTGGCCCTGCAGCAGACCATGTATGAAATGGGCCGTGCGGTGA
TTGAAACCCATCCGGAAATCGATGAAATCAAAATGAGCCTGCCGAACAAA
CATCATTTTCTGGTGGATCTGCAGCCGTTTGGCCAGGATAATCCGAACGA
AGTGTTTTATGCGGCGGATCGTCCGTATGGCCTGATTGAAGCGACCATTC
AGCGTGAAGGCAGCCGTGCGGATCATCCGATTTGGAGCAACATTGCGGGC
TTTTGCTAA
URIC_PICJA (Geneart # 0709653):
ATGAGCACCACCCTGAGCAGCAGCACCTATGGCAAAGATAACGTGAAATT
CCTGAAAGTGAAAAAAGATCCGCAGAATCCGAAAAAACAAGAAGTGATGG
AAGCGACCGTGACCTGCCTGCTGGAAGGCGGCTTTGATACCAGCTATACC
GAAGCGGATAACAGCAGCATTGTGCCGACCGATACCGTGAAAAACACCAT
TCTGGTGCTGGCCAAAACCACCGAAATTTGGCCGATTGAACGTTTTGCGG
CGAAACTGGCCACCCATTTCGTGGAAAAATATTCTCATGTGAGCGGCGTG
TCTGTGAAAATTGTGCAGGATCGCTGGGTGAAATATGCGGTGGATGGCAA
ACCGCATGATCATAGCTTTATTCATGAAGGCGGCGAAAAACGTATTACCG
ATCTGTATTATAAACGCAGCGGCGATTATAAACTGAGCAGCGCGATTAAA
GATCTGACCGTGCTGAAAAGCACCGGCAGCATGTTTTATGGCTATAACAA
ATGCGATTTTACCACCCTGCAGCCGACCACCGATCGTATTCTGAGCACCG
ATGTGGATGCGACCTGGGTGTGGGATAACAAAAAAATCGGCAGCGTGTAT
GACATTGCGAAAGCGGCGGATAAAGGCATTTTCGATAACGTGTATAACCA
GGCGCGTGAAATTACCCTGACCACCTTTGCGCTGGAAAACAGCCCGAGCG
TGCAGGCGACCATGTTTAACATGGCGACCCAGATTCTGGAAAAAGCGTGT
AGCGTGTATAGCGTGAGCTATGCGCTGCCGAACAAACATTACTTTCTGAT
CGATCTGAAATGGAAAGGCCTGGAAAACGATAACGAACTGTTTTATCCGA
GCCCGCATCCGAACGGCCTGATTAAATGCACCGTGGTGCGTAAAGAAAAA
ACCAAACTGTAA
URIC_PSEAE (Geneart #0709654):
ATGCCGAAAAGCAGCGCGGCGGAACAGAGCGGTGAAAGCAGCACCCAGAC
CCTGAGCCTGCTGGATGAAATTATTGCGAAAGGCCGTATGGCGCATGATG
ATAGCCAGCAGGATTATGCGCGTGATATGCTGGCCGAATTTGCGACCCAG
GTGCTGGATGAAGGCATGGCGGTGGATAAAGATACCGTGGCGATGATTAA
CGATCGTATCAGCCAGATTGATGCGCTGATTAGCGATCAGCTGAACCAGA
TTATTCATCATCCGGAACTGCAGAAACTGGAAGCGAGCTGGCGTGGCCTG
CATCAGCTGGTGAGCAACACCGAAACCAGCGCGCGTCTGAAACTGCGTCT
GCTGAACGTGGGCAAAAACGAACTGCAGAACGATCTGGAAAAAGCGGTGG
AATTTGATCAGAGCGCGCTGTTCAAAAAAATCTACGAAGAAGAATACGGC
ACCTTTGGCGGCCAGCCGTTCAGCCTGCTGATTGGCGATTTTACCTTTGG
CCGTCATCCGCAGGATATTGGCCTGCTGGAAAAACTGAGCAACGTGGCGG
CGGCCGCACATGCACCGTTTATTGCGGCGGCGAGCCCGCGTCTGTTTGAT
ATGAACAGCTTTACCGAACTGGCCGTGCCGCGTGATCTGACCAAAATTTT
CGAAAGCCTGGAACTGATTAAATGGCGTGCGTTTCGTGAAAGCGAAGATA
GCCGTTATGTGAGCCTGGTGCTGCCGAACTTTCTGCTGCGTCTGCCGTAT
GGCCCGGAAACCCGTCCGGTGGAAGGCATGAACTATGTGGAAGATGTGAA
CGGCACCGATCATAGCAAATATCTGTGGGGCAACGCGGCGTGGGTTCTGG
CCCAGCGTATTACCGAAGCGTTTGCGAAATATGGCTGGTGCGCGGCGATT
CGTGGTGCGGAAGGCGGTGGTGCGGTTGAAGGTCTGCCGGCGCATACCTT
TCGTACCAGCAGCGGCGATCTGAGCCTGAAATGCCCGACCGAAGTGGCGA
TTACCGATCGTCGTGAAAAAGAACTGAACGATCTGGGCTTTATTAGCCTG
TGCCACAAAAAAAACAGCGATGTGGCGGTGTTTTTTGGCGGTCAGACCAC
CAACAAAGCGCGTCTGTATAACACCAACGAAGCGAACGCGAACGCGCGTC
TGAGCGCGATGCTGCCGTATGTTCTGGCCGCGAGCCGTTTTGCGCATTAT
CTGAAAGTGATCATGCGTGATAAAGTGGGCAGCTTTATGACCCGTGATAA
CGTGCAGACCTATCTGAACAACTGGATTGCGGATTATGTGCTGATTAACG
ATAACGCGCCGCAGGAAATCAAAGCGCAGTATCCGCTGCGTGAAGCGCGT
GTGGATGTGAGCGAAGTGGCGGGCAAACCGGGTGCGTATCGTGCGACCGT
GTTTCTGCGTCCGCATTTTCAGCTGGAAGAACTGAGCGCGAGCATTCGTC
TGGTGGCGAATCTGCCGCCGCCGGTTGCGGCGTAA

Uricase Expression Vectors for E. coli

PCR reactions to amplify the uricase gene from the Geneart template plasmids generated significant products of the correct sizes for the expected genes as analyzed by running the products on a 1% agarose gel. Table 5 contains a list of the genes and the number of base pairs in each gene.

TABLE 5
Uricase gene sizes in base pairs (bp)

| CONSTRUCT NAME | SIZE (BP) |
|---|---|
| URIC1_SOYBN | 930 bp |
| URIC_PHAVU | 928 bp |
| URIC_ARTGL | 909 bp |
| URIC_PICJA | 912 bp |
| URIC_PSEAE | 1485 bp |

The PCR products were purified, digested and ligated into pET9a and pET11a as described in the Experimental methods section. Transformation of the ligation reactions was very efficient with a minimum of 4× the number of colonies for the ligation versus the control plate where no insert was added. Either 2 or 4 single colonies were picked from each plate to screen for positive clones containing the uricase gene. Screening was performed by Pst I digest and analysis of the digests by agarose gel electrophoresis.

Table 6 contains a list of the expression vectors that have been generated and the uricase gene in each has been sequenced and confirmed.

TABLE 6
Uricase expression vectors

| VECTORS HAVING | VECTORS HAVING |
|---|---|
| AMPICILLIN RESISTANCE | KANAMYCIN RESISTANCE |
| pET11a - URIC1_SOYBN | pET9a - URIC1_SOYBN |
| pET11a - 6 × His-URIC1_SOYBN | pET9a - 6 × His-URIC1_SOYBN |
| pET11a - URIC_PHAVU | pET9a - URIC_PHAVU |
| pET11a - 6 × His-URIC_PHAVU | pET9a - 6 × His-URIC_PHAVU |
| pET11a - URIC_ARTGL | pET9a - URIC_ARTGL |
| pET11a - 6 × His-URIC_ARTGL | pET9a - 6 × His-URIC_ARTGL |
| pET11a - URIC_PICJA | pET9a - URIC_PICJA |
| pET11a - 6 × His-URIC_PICJA | pET9a - 6 × His-URIC_PICJA |
| | pET9a - 6 × His-URIC_PSEAE |

### Small Scale Evaluation of Uricase Expression and Activity

For initial proof-of-concept studies, C. utilis uricase (URIC_PICJA) was chosen as the enzyme best suited for oral delivery. This selection was based on extensive characterization of two commercially available C. utilis uricase enzymes. The first enzyme is the native C. utilis uricase available from Amano enzyme and the second is a recombinant C. utilis enzyme available from Biozyme. Preliminary experiments to check expression and activity of the other candidates have suggested that the Candida utilis enzyme is the best candidate. Although the P. aeruginosa enzyme is reported to have >600 U/mg activity, in preliminary experiments, the enzyme expressed in E. coli has no detectable activity. We need to test the native enzyme secreted from P. aeruginosa before ruling out this candidate.

Selection of the Candida utilis Uricase Clone for Scale Up

Expression of untagged Candida utilis uricase from the two different expression vectors was compared as the first step in choosing a clone for scale up. In the first experiment, uricase expression was induced in BL21(DE3) cells transformed with either pET9a-URIC_PICJA or pET11a-URIC_PICJA. Cells harvested after induction were lysed, analyzed by SDS-PAGE and the uricase activity assay. Both of the vectors supported expression of soluble uricase as is apparent by SDS-PAGE analysis (data not shown). Overall we observed that the cells grown in the presence of kanamycin grew more slowly than those in ampicillin. Thus, at the end of the experiment the cell number was lower (by about half) in the case of the kanamycin culture compared to the ampicillin culture. The gel was loaded to correct for the differences in cell number. The gel shows that the expression levels are very similar between the two vectors/strains when comparing an equivalent number of cells. The cultures grown in the presence of kanamycin would take longer to grow and accumulate to the same cell density as the cultures grown in the presence of ampicillin. If the same cell density is reached, the same amount of uricase would be generated by the cultures grown in ampicillin or kanamycin.

Because the levels of uricase expression were similar between the two clones we decided to screen 6 clones from each type (pET11a Amp and pET9a Kan) to identify the best clone to proceed with large scale fermentation. Uricase runs at its expected size of 34 kDa (monomer) (data not shown). The activity measurements (initial rates) for pET9a and pET11a are shown in Table 7 and Table 8, respectively. Again, we observed that the kanamycin resistant strain grew more slowly than the ampicillin resistant strain. All of the ampicillin cultures were ready to induce 30-45 min before the kanamycin cultures. A small amount of each starter culture was spotted onto selective media to keep for future use. Cell numbers for the KAN and AMP strains after the 3 hour induction were very close. Still, the same number of cells was harvested for each of the cultures so the expression levels could be adequately compared.

TABLE 7
Uricase activity (initial rates) for pET9a Candida utilis clones

| | pET9A + C. UTILIS URICASE |
|---|---|
| 1 | 0.50 |
| 2 | 0.62 |
| 3 | 0.41 |
| 4 | 0.51 |
| 5 | 0.47 |
| 6 | 0.37 |

TABLE 8
Uricase activity (initial rates) for pET11a Candida utilis clones

| | pET11A − C. UTILIS URICASE |
|---|---|
| 1 | 0.56 |
| 2 | 0.43 |
| 3 | 0.56 |
| 4 | 0.58 |
| 5 | 0.49 |
| 6 | 0.62 |

All of the activities and expression levels for all of the clones were very similar, as judged by SDS-PAGE. For pET9a, colony #2 was selected and for pET11a, colony #6 was selected. These colonies were cultured overnight and then stored in 20% glycerol, flash frozen in liquid nitrogen and then stored at −80° C.

### Conclusions

Expression constructs for uricase enzymes from five different organisms have been generated. These enzymes were selected based on diversity and their potential for high activity and stability in the GI tract. Candida utilis (URIC_PICJA) uricase was chosen for the first scale up fermentation run. The remaining uricase constructs will require further analysis to confirm their individual specific activities and expression levels.

### Example 15

### Characterization of Uricases

### Objective

Uricases from different sources were characterized for their suitability as oral drug candidates for the treatment of Gout. Ideally, an oral drug has stability in low gastric pH, activity at pHs 7.5 and below and stability against proteases. Crystallization and formulation of Uricases were pursued for that reason but characterization was performed to gain knowledge of the strengths and weaknesses of each Uricase tested in these experiments. Parameters checked were purity, activity at different pHs, pH stability, and stability against proteases.

### Equipment and Material

### Equipment

Mini gel apparatus: Invitrogen, Cat # EI0001
Power supply: Bio-RAD, Model 1000/500
Heating blocks (37-99° C. with 0.5 ml and 1.5 ml tube holders): Eppendorf thermo mixer R

### UV Spectrophotometer: Agilent, Model # 8453

Circulating water bath: Cole-Parmer, Model # 12108-10
UV transparent disposable cuvettes Macro: Fisher, Cat # 14-377-009
UV transparent disposable cuvettes semi-micro: Fisher, Cat # 13-688-73
Vortex mixer: Fisher, Cat # 02215370
Balance: Denver instrument Company, Model # A-200DS
pH meter: Fisher, Model; Accumet Basic AB15

### Centrifuge: Eppendorf Model 5415C

Orbital shaker platform: Scienceware, Cat # F37041-0000
Magnetic stirrer: Corning, Model PC 410
Gel staining tray: Invitrogen, Cat # NI 2400
Gel opening knife: Invitrogen, Cat # EI9010
Gel loading tips: Fisher, Cat # LC 1001

### Material

### NOVEX® 4-20% TRIS-GLYCINE PRE CAST MINI GELS, 1.0 mM, 10 WELLS: INVITROGEN, CATALOG # EC 6025BOX

SDS PAGE running buffer (10×): Invitrogen, Catalog #LC2675-5
SDS PAGE sample buffer (2×): Invitrogen, Catalog #LC2676
NOVEX® Sharp unstained protein standard: Invitrogen, Catalog #LC5801
NOVEX® pH 3-10 IEF gels, 1.0 mm, 10 wells: Invitrogen, Catalog #EC 6655BOX
pH 3-10 IEF buffer kit: Invitrogen, Cat # LC 5317
IEF standards for p14.45-9.6: Bio-RAD, Cat # 161-0310
NUPAGE® Novex® 4-12% Bis Tris gels 1.0 mm, 10 wells: Invitrogen, Cat # NP 0321BOX
NUPAGE® MES SDS buffer kit: Invitrogen, Cat # NP0060
Coomassie stain: Bio-RAD, Cat # 161-7878EDU
β-mercapto-ethanol: Sigma, Catalog #M 6250, Lot # 119H0914
Coomassie blue R 250: Sigma, Catalog #B 0149, Lot # 13H5002

### Methanol: Fisher, A452-4, Lot # 061495

Glacial Acetic acid: Fisher, Catalog #A38C-212, Lot # 061401

### Tris Base: Fisher, Catalog #BP152-5

1N Hydrochloric acid: Fisher, Catalog #SA48-500

### DL Dithiothreitol: Sigma, Catalog #D0632-100G

Sulfosalicylic acid: Fisher, Cat # A297-500
Trichloroacetic acid: Fisher, Cat # 324-500
10N Hydrochloric acid: Fisher, Cat # SA49

### Boric Acid: Sigma, Cat # B0394-500G

Sodium hydroxide: Fisher, Cat # AC42433-5000

### Uric Acid: Sigma, Cat # U-0881

1N Sodium hydroxide: Fisher, Cat # AC12426-0010

### Histidine: Sigma, Cat # H-8125

Aspartic acid: Fluka, Cat # 11189

### Cysteine: Fluka, Cat # 30090

### Chymotrypsin: Sigma, Cat # C 3142-100MG

### Trypsin: Sigma, Cat # T-7309

### Uricases

Amano Uricase: Amano, Cat # UR-2, Source Candida utilis
Biozyme Uricase: Biozyme, Cat # U5, Source Recombinant E. coli expressed enzyme Original cDNA from Candida species
Genzyme Uricase: Genzyme, Cat # 1701, Source Bacillus fastidiosus
Uricase T-129: Genzyme, Cat # T-129, Source Arthrobacter globiformis
Rasburicase (Elitek): Sanofi-Aventis, NDC 0024-5150-10, Source Recombinant Saccharomyces cerevisiae expressed enzyme Original cDNA from Aspergillus flavus
Fluka Uricase: Fluka, Source Bacillus fastidiosus
In-house Uricase: Altus Uricase, Source Recombinant E. coli expressed enzyme Original cDNA from Candida utilis

### Uricase Characterization

### Molecular Weight Determination

Molecular weight was determined using SDS PAGE. 4-20% Tris glycine gels were used. Samples were reduced with β-mercapto ethanol. 10 μg of protein was loaded per well for all the samples. The molecular weight of the monomer for Amano, Biozyme, T-129 and Rasburicase was approximately 32 kDa which is the theoretical mass for Uricase E.C 1.7.3.3. Biozyme had an additional band between 30 and 20 kDa. Genzyme and Fluka Uricases had slightly higher molecular weights than other Uricases and contained an additional band between 60 and 80 kDa. A standard procedure was used for running SDS PAGE.

### Iso-Electric Point Determination

pH 3-10 IEF gels were used for determination of pI. 20 μg of Amano and Biozyme Uricase, 5 μg of Genzyme, T-129, Rasburicase and Fluka Uricase and 15 μg of In-house Uricase were loaded on the gel. pI of the protein is important to determine charge on the molecule at a given pH. Uricases from different sources had different pI profile and some of them were not matching the literature value or the theoretical value for the pI. IEF gel was performed using the standard procedure.

### pH Profile for Activity

Uricase activity was tested using boric acid/NaOH buffer at different pHs. Activity was tested at pH 6, 7, 8, 9 and 10. The highest pH in the GI tract is approximately 7.5 but Uricases have a pH optimum for activity around 8.5, so pHs 8, 9 and 10 were also tested. pHs below 6 were not tested as uric acid is poorly soluble at acidic pHs and this makes it difficult to make substrate solution at lower pHs. The activity assay was performed in kinetics mode by monitoring the decrease in absorbance at 292 nm for 5 minutes at 37° C.

### Assay Procedure:

50 mM Boric acid buffers were made by dissolving 3.1 grams of boric acid in 950 ml of water. Adjusted pH to 6.0, 7.0, 8.0, 9.0 or 10.0 with 4 N NaOH and volume was adjusted to 1000 ml. pH was checked and adjusted again if necessary. Buffer pH was read without stirring.

0.13 mM Uric acid (Substrate solution) was made by dissolving ˜10 mg of uric acid in an appropriate amount of 50 mM Boric acid buffer for each pH 6 to 10 to make the concentration 1.3 mM (21.85 mg/100 ml). Diluted 1.3 mM uric acid solution 10 times to make it 0.13 mM using the same pH 50 mM Boric acid buffer. pH was checked and adjusted again to original value if necessary. (e.g. substrate solution of pH 10 was made by dissolving uric acid in 50 mM Boric acid buffer pH 10 to make it 1.3 mM and then diluted 10 times using 50 mM Boric acid buffer pH 10. The pH of the 0.13 mM substrate solution was checked and adjusted again to pH 10 if needed)

Set up spectrophotometer for the assay method (Uricase.m) at following parameters:

Mode: Kinetics mode

Absorbance: 292 nm

Read Time: 0-300 seconds at interval of 5S

Calculation for Rate: Initial rate for 0-300 seconds

Multiply by −60 to get AU/min

Circulating water bath was attached to multi-cell cuvette holder of the spectrophotometer for temperature control and set to 37° C.

Took 3 ml of 50 mM Boric acid buffer pH 6.0 in a UV transparent disposable cuvette and blanked the spectrophotometer.

3 ml of uric acid solution at pH 6.0 was taken in cuvettes with tiny magnets for stirring. Solution was equilibrated to 37° C.

20 μl of 50 mM Boric acid buffer pH 6.0 was added to the substrate for the assay blank and samples at 0.05 mg/ml concentration by A280 for test. Kinetic assay was started and rate/min was obtained. (Sample and blank volume were changed based on protein concentration or activity units)

Printed results and calculated activity.

Repeated assay for pH 7.0, 8.0, 9.0, 10.0 with appropriate blank for spectrophotometer and blank for the assay.

### pH Stability:

pH stability was measured by incubating Amano, Biozyme and In-house Uricases in different pH buffers and then testing activity at pH 8.0. Activity was measured using the standard activity assay procedure. pH stability is important as the drug has to pass through and survive low gastric pH before it goes further down in the GI tract where pH is favorable for activity. All enzymes have a similar profile for pH stability but the Amano and In-house enzyme has higher units compared to Biozyme.

### Assay Procedure:

388 mg of Histidine, 332.8 mg of Aspartic acid and 302.9 mg of Cysteine were dissolved in 100 ml of water to make 25 mM concentration of each amino acid.

10 ml of this solution was taken in 8 different beakers and the pH was adjusted with 1 N HCl or 1 N NaOH to 2.0, 3.0, 4.0, 5.0, 6.0, 7.0, 8.0 or 9.0. pH reading was without stirring.

28.64 mg/ml Amano Uricase was diluted to 1 mg/ml using the appropriate pH buffer at the time specified in the Table below while starting a timer to read time up from 0. Mixed and took 10 μl samples at 0 and 2 hours as shown in Table. Samples were diluted with 90 μl of 25 mM Tris pH 7.5 to make the concentration 0.1 mg/ml. Mixed nicely and assayed.

34.54 mg/ml Biozyme Uricase was diluted to 1 mg/ml using the appropriate pH buffer at the time specified in Table 9 below. Mixed and took 10 μl samples at 0 and 2 hours as shown in Table 9. Samples were diluted with 90 μl of 25 mM Tris pH 7.5 to make the concentration 0.1 mg/ml. Mixed nicely and assayed.

All samples were incubated in a heating block at 37° C. with shaking at 400 rpm for incubation.

The control was made by 10 fold dilution of 1.0 mg/ml protein samples that were in 10 mM Tris pH 7.5 with 25 mM Tris buffer pH 7.5 (10 μl sample+90 μl buffer) and assayed before starting samples for different pHs. Then incubated at 37° C. until all the time points were done and tested again after 4 hours.

TABLE 9
Scheme for pH stability sample time:

| | | | SAMPLES TAKEN AT FOR | | ASSAY PERFORMED AT | |
|---|---|---|---|---|---|---|
| SAMPLE | pH | URICASE | 0 time | 2 hours | For 0 H | For 2 H |
| 418-92-1A | 2.0 | Amano | 00:00:00 | 02:00:00 | 00:00:38 | Within 20 |
| 418-92-1B | 2.0 | Biozyme | 00:15:00 | 02:15:00 | 00:15:38 | seconds |
| 418-92-2A | 3.0 | Amano | 00:30:00 | 02:30:00 | 00:30:42 | |
| 418-92-2B | 3.0 | Biozyme | 00:45:00 | 02:45:00 | 00:45:36 | |
| 418-92-3A | 4.0 | Amano | 01:00:00 | 03:00:00 | 01:00:38 | |
| 418-92-3B | 4.0 | Biozyme | 01:15:00 | 03:15:00 | 01:15:40 | |
| 418-92-4A | 5.0 | Amano | 01:30:00 | 03:30:00 | 01:30:30 | |
| 418-92-4B | 5.0 | Biozyme | 01:45:00 | 03:45:00 | 01:45:37 | |

| | | | SAMPLES TAKEN AT FOR | | FOR 0 H | FOR 2 H |
|---|---|---|---|---|---|---|
| SAMPLE | pH | URICASE | 0 time | 2 hours | Assay was done after | |
| 499-1-1A | 6.0 | Amano | 00:00:00 | 02:00:00 | 00:00:31 | 02:00:32 |
| 499-1-1B | 6.0 | Biozyme | 00:15:00 | 02:15:00 | 00:15:35 | 02:15:30 |
| 499-1-2A | 7.0 | Amano | 00:30:00 | 02:30:00 | 00:30:36 | 02:30:32 |
| 499-1-2B | 7.0 | Biozyme | 00:45:00 | 02:45:00 | 00:45:35 | 02:45:23 |
| 499-1-3A | 8.0 | Amano | 01:00:00 | 03:00:00 | 01:00:37 | 03:00:16 |
| 499-1-3B | 8.0 | Biozyme | 01:15:00 | 03:15:00 | 01:15:38 | 03:15:13 |
| 499-1-4A | 9.0 | Amano | 01:30:00 | 03:30:00 | 01:30:38 | 03:30:19 |
| 499-1-4B | 9.0 | Biozyme | 01:45:00 | 03:45:00 | 01:45:29 | 03:45:17 |

Table 9 describes time for starting the incubation for Amano or Biozyme enzymes with different pH buffers for stability testing. Mixing enzymes at different time for different pH made it possible to accurately assay activity after 2 hours for each enzyme at each pH tested.

Assay was performed using 0.13 mM Uric acid substrate made with 50 mM Boric acid buffer pH 8.0

50 mM Boric acid buffer was made by dissolving 3.1 grams of boric acid in 950 ml of water. Adjusted pH to 8.0 with 4 N NaOH and volume was adjusted to 1000 ml. pH was checked and adjusted again if necessary. Buffer pH was read without stirring.

0.13 mM Uric acid (Substrate solution) was made by dissolving ˜10 mg of uric acid in appropriate amount of 50 mM Boric acid buffer pH 8.0 to make it 1.3 mM concentration (21.85 mg/100 ml). Diluted 1.3 mM uric acid solution 10 times to make the concentration 0.13 mM using 50 mM Boric acid buffer pH 8.0. pH was checked and adjusted again if necessary.

Set up spectrophotometer for the assay method (Uricase.m) at following parameters:

Mode: Kinetics mode

Absorbance: 292 nm

Read Time: 0-300 seconds at interval of 5S

Calculation for Rate: Initial rate for 0-300 seconds

Multiply by −60 to get AU/min

Circulating water bath was attached to multi-cell cuvette holder of the spectrophotometer for temperature control and set to 37° C.

Took 3 ml of 50 mM Boric acid buffer pH 8.0 in a UV transparent disposable cuvette and blanked the spectrophotometer.

3 ml of uric acid solution at pH 8.0 was taken in cuvettes with tiny magnets for stirring. Solution was equilibrated to 37° C.

10 μl of 50 mM Boric acid buffer pH 8.0 was added to the substrate for the assay blank and samples at 0.1 mg/ml concentration by A280 for test. Kinetic assay was started and rate/min was obtained. (Sample and blank volume were changed based on protein concentration or activity units)

Printed results and calculated activity.

### Protease Stability

A drug that is designed to work in GI tract has to be stable against proteases in order to perform. Three main proteases are pepsin, trypsin and chymotrypsin. Pepsin works at low pH around pH 2.0 and trypsin as well as chymotrypsin work at neutral pH around 7.5. Since Uricases are not stable at pH 2.0 it was not possible to do protease stability for pepsin.

### Assay Procedure:

Tubes were labeled appropriately for trypsin and chymotrypsin stability samples as well as controls.

150 μl of appropriate sample (Amano, Biozyme or Genzyme Uricase) was taken at 1.0 mg/ml concentration in each tube.

For trypsin stability, added 3 μl of 1 mg/ml trypsin to tubes at the time mentioned in the table and mixed nicely. 10 μl of sample was taken at different time points as in Table 10 below and added to 90 μl of 10 mM Tris pH 7.5 to dilute samples to 0.1 mg/ml for the assay. Mixed nicely and assayed.

For chymotrypsin stability, added 3 μl of 1 mg/ml chymotrypsin to tubes at the time mentioned in the table and mixed nicely. 10 μl of sample was taken at different time points as in the Table 10 below and added to 90 μl of 10 mM Tris pH 7.5 to dilute samples to 0.1 mg/ml for the assay. Mixed nicely and assayed.

For Controls, added 3 μl of 25 mM Tris pH 7.5 to tubes at the time mentioned in Table and mixed nicely. Took 10 μl of sample for different time points as in the table below and added to 90 μl of 10 mM Tris pH 7.5. Mixed nicely and assayed.

All samples were incubated in a heating block at 37° C. with shaking at 400 rpm. Samples were taken after 1.5 hour, 3 hours and 4.5 hours for activity assay.

TABLE 10
Example scheme for protease stability

| SAMPLE | PROTEASE | 0 TIME | 1.5 HOUR | 3 HOURS | 4.5 HOURS |
|---|---|---|---|---|---|
| Amano | Trypsin | 0 min | 1 hour 30 min | 3 hour 0 min | 4 hour 30 min |
| Biozyme | Trypsin | 15 min | 1 hour 45 min | 3 hour 15 min | 4 hour 45 min |
| Amano | Chymotrypsin | 30 min | 2 hour 0 min | 3 hour 30 min | 5 hour 0 min |
| Biozyme | Chymotrypsin | 45 min | 2 hour 15 min | 3 hour 45 min | 5 hour 15 min |
| Amano | None | 1 hour 0 min | 2 hour 30 min | 4 hour 0 min | 5 hour 30 min |
| Biozyme | None | 1 hour 15 min | 2 hour 45 min | 4 hour 15 min | 5 hour 45 min |

Table 10 has an example plan for testing stability against proteases. It is timed to accurately measure activity of more than one enzyme for multiple proteases in single experiment.

Assay was performed using 0.13 mM Uric acid substrate made with 50 mM Boric acid buffer pH 8.0

50 mM Boric acid buffer was made by dissolving 3.1 grams of boric acid in 950 ml of water. Adjusted pH to 8.0 with 4 N NaOH and volume was adjusted to 1000 ml. pH was checked and adjusted again if necessary. Buffer pH was read without stirring.

0.13 mM Uric acid (Substrate solution) was made by dissolving ˜10 mg of uric acid in appropriate amount of 50 mM Boric acid buffer pH 8.0 to make it 1.3 mM (21.85 mg/100 ml). 1.3 mM uric acid solution was diluted 10 times to make it 0.13 mM using 50 mM Boric acid buffer pH 8.0. pH was checked and adjusted again if necessary.

Set up spectrophotometer for the assay method (Uricase.m) at following parameters

Mode: Kinetics mode

Absorbance: 292 nm

Read Time: 0-300 seconds at interval of 5 seconds

Calculation for Rate: Initial rate for 0-300 seconds

Multiply by −60 to get AU/min

Circulating water bath was attached to the multi-cell cuvette holder of the spectrophotometer for temperature control and set to 37° C.

Took 3 ml of 50 mM Boric acid buffer pH 8.0 in a UV transparent disposable cuvette and blanked the spectrophotometer.

3 ml of uric acid solution at pH 8.0 was taken in cuvettes with tiny magnets for stirring. Solution was equilibrated to 37° C.

10 μl of 50 mM Boric acid buffer pH 8.0 was added to the substrate for the assay blank and samples at 0.1 mg/ml concentration by A280 for test. Kinetic assay was started and rate/min was obtained. (Sample and blank volume were changed based on protein concentration or activity units)

Printed results and calculated activity.

### Results

### Molecular Weight Determination

Molecular weight of different Uricases was determined by visual comparison of sample bands with the standard loaded on the gel (data not shown). Standard was NOVEX® sharp unstained protein standard from Invitrogen. In-house Uricase was analyzed using 4-12% Bis-Tris NUPAGE® SDS gel using See blue 2 plus Marker. Rasburicase and In-house Uricase are the most pure Uricases. Rasburicase is commercial drug and was used to compare purity of other Uricases not as a candidate for crystallization. Amano and T-129 Uricases have minor impurities. In-House Uricase as well as Amano and T-129 are best candidates for crystallization based on purity. Biozyme, Genzyme and Fluka Uricases show more impurities compared to other Uricases on the gel.

### pI Determination

pI of different Uricases was determined by visual comparison of sample bands with the standard loaded on the gel (data not shown). IEF gel for In-house Uricase was performed using pH 3-10 IEF gel. Amano and In-house Uricases had multiple bands of similar intensity between pH 6.0 to 8.0. Biozyme showed a major band close to pH 5.1 with some minor bands above and below pH 5.1. Genzyme Uricase and Fluka Uricase had a band close to pH 6.0 and a minor band between pH 5.1 and 4.5. T-129 Uricase had one band close to pH 5.1. Rasburicase had two bands one major and one minor band close to pH 8.0. Based on the pI profile difficulty to crystallize can be ranked as Amano, In-house>Biozyme>Genzyme, Fluka and Rasburicase>T-129.

### pH Profile for Activity

Activity for all Uricases was determined using kinetics mode at 37° C. Protein concentration for all Uricases except Rasburicase was determined by A₂₈₀ considering the extinction coefficient as 1. For rasburicase protein concentration was determined based on vial label for protein content and final volume after re-suspension. pHs tested were 6, 7, 8, 9 and 10. A molar extinction coeffient of 12.3 was used for activity calculations. Activity was calculated using following equation:

$$Activity U \text{/} mg = \frac{( δ Abs \text{/} min of sample - δ Abs \text{/} min of blank ) * total volume ( ml )}{12.3 * sample volume ( ml ) * sample concentration ( mg \text{/} ml )}$$

Activity for in-house Uricase was done separately. Original experiment for pH profile gave activity of 33.7 units/mg but it was the only time this value was observed. Average activity for in-house Uricase at pH 8.0 was found to be between 23-27 U/mg when assayed for different reasons. So activity for pH 8.0 was considered as 25.09 from another experiment which is representative for most of the experiments.

Based on pH profile the best Uricases were Amano and in-house Uricase. Both Uricases have broad range of pH for activity with good activity between pH 6 to 7 compared to other enzymes which is important for functioning in GI tract.

TABLE 11
Activity data at different pHs

| pH | AMANO | BIOZYME | GENZYME | T-129 | RASBURICASE | FLUKA | IN HOUSE |
|---|---|---|---|---|---|---|---|
| 6.00 | 16.06 | 11.48 | 2.07 | 0.09 | 7.73 | 0.04 | 16.60 |
| 7.00 | 21.15 | 12.66 | 5.70 | 8.25 | 26.65 | 3.72 | 23.70 |
| 8.00 | 19.81 | 11.95 | 10.95 | 28.36 | 32.42 | 11.39 | 25.09 |
| 9.00 | 13.66 | 7.69 | 15.33 | 29.71 | 20.88 | 13.85 | 20.80 |
| 10.00 | 5.66 | 3.44 | 7.51 | 14.40 | 8.63 | 5.45 | 7.60 |

Table 11 compares the activities of different Uricases at different pHs. In the assay conditions used the optimum pH for activity for Amano and Biozyme Uricase is 7, for Genzyme, T-129 and Fluka Uricases it is 9 and for Rasburicase and In-house Uricase it is 8.

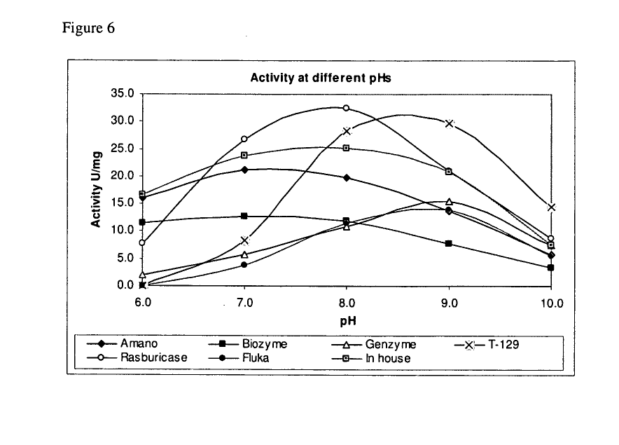

FIG. 6 shows the activity profiles at different pHs for different Uricases. The figure shows Rasburicase having maximum activity at pH 8 but a significant drop in activity at pH 6.0. Amano and In-house Uricase have good profiles between pH 6 to 8 and Biozyme with similar profile but less activity units.

### pH Stability

pH stability was tested for pHs between 2-9 in increments of 1 pH unit. Different pH incubation buffers were made using amino acids. Buffers were made with 3 amino acids, 25 mM Histidine, 25 mM Aspartic acid and 25 mM Cysteine. Activity was tested at time zero and after 2 hour incubation at 37° C. for each pH. Activity was expressed in two ways; Actual units remaining after incubation time or % activity remained compared to activity of control (0H activity for pH 7.0 was control for all 0H activities for different pHs and 2H activity for pH 7.0 was control for all 2H activities for other pHs). pH stability for In-house Uricase was determined a using same experimental method. Based on pH stability all three Uricases are unstable at pHs below 6 and have to be formulated for use as an oral drug for functioning in GI tract.

TABLE 12
pH stability data

| U/MG | pH 2.0 | pH 3.0 | pH 4.0 | pH 5.0 | pH 6.0 | pH 7.0 | pH 8.0 | pH 9.0 |
|---|---|---|---|---|---|---|---|---|
| Amano 0 H | 0.00 | 3.28 | 11.93 | 20.96 | 26.70 | 27.06 | 26.50 | 26.27 |
| Amano 2 H | 0.00 | 0.00 | 0.00 | 14.46 | 25.63 | 26.55 | 27.81 | 25.45 |
| Biozyme 0 H | 0.00 | 0.04 | 2.23 | 10.01 | 12.10 | 12.55 | 11.57 | 12.68 |
| Biozyme 2 H | 0.00 | 0.00 | 0.00 | 5.82 | 12.12 | 11.83 | 12.20 | 13.78 |
| In-house 0 H | 0.49 | 4.16 | 8.08 | 21.59 | 22.27 | 26.18 | 25.94 | 21.29 |
| In-house 2 H | 0.00 | 0.00 | 0.24 | 10.47 | 23.49 | 25.94 | 26.43 | 26.92 |
| % ACTIVITY | pH 2.0 | pH 3.0 | pH 4.0 | pH 5.0 | pH 6.0 | pH 7.0 | pH 8.0 | pH 9.0 |
| Amano 0 H | 0% | 12% | 44% | 77% | 99% | 100% | 98% | 98% |
| Amano 2 H | 0% | 0% | 0% | 54% | 97% | 100% | 105% | 105% |
| Biozyme 0 H | 0% | 0% | 18% | 80% | 96% | 100% | 92% | 101% |
| Biozyme 2 H | 0% | 0% | 0% | 49% | 102% | 100% | 103% | 117% |
| In-house 0 H | 2% | 16% | 31% | 82% | 85% | 100% | 99% | 81% |
| In-house 2 H | 0% | 0% | 1% | 40% | 91% | 100% | 102% | 104% |

Table 12 describes pH stability for Amano, Biozyme and In-house Uricases at pHs 2 to 9. All enzymes are stable for 2 hours at 37° C. at pH 6 and above but lose activity rapidly below pH 6. Top table is activity in U/mg and bottom table expresses activity as % remaining compared to control.

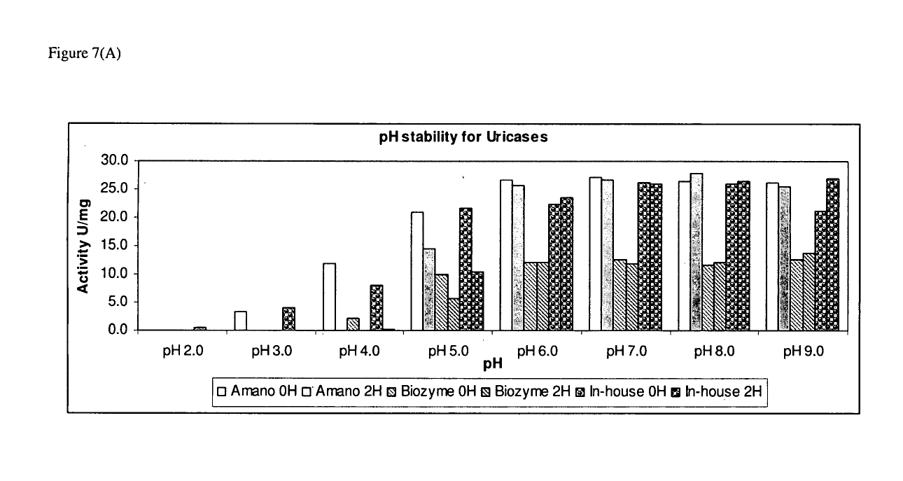

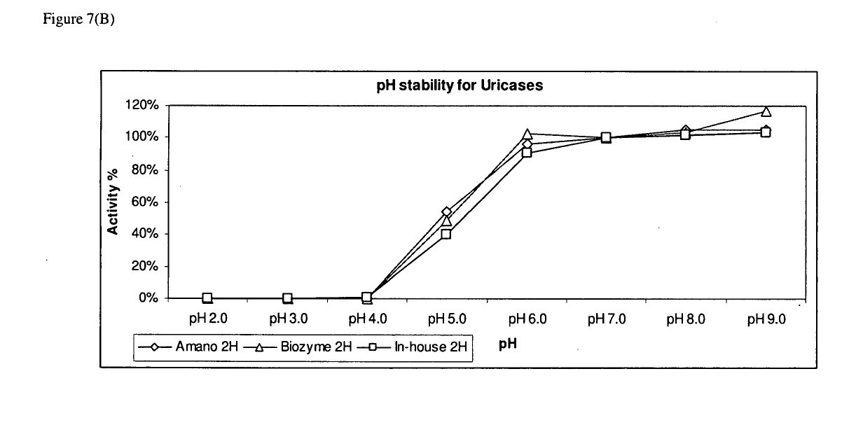

FIGS. 7(A) and 7(B) show pH stability comparisons. FIG. 7(A) shows pH stability at different pHs for different Uricases in Units/mg. The figures show more remaining activity for Amano Uricase and In-House Uricase compared to Biozyme. FIG. 7(B) shows pH stability in terms of % remaining activity at 2 hours compared to control activity at 2 hour time point. Amano, Biozyme and In-house Uricases are similar in profile being stable at pH 6 and above and unstable at pHs below 6.

### Protease Stability

Protease stability was tested using trypsin and chymotrypsin proteases. Activity was expressed as actual units remaining and also as % remaining compared to 0H time point. Amano, Biozyme and In-house Uricases were tested against Trypsin stability and all were stable for 4 to 4.5 hours at 37° C. Amano, Biozyme Genzyme and in-house Uricases were tested against Chymotrypsin for stability. Amano and In-house Uricases were stable against chymotrypsin while Biozyme and Genzyme Uricase were not stable compared to the other two. Amano and In-house Uricases are best candidates for protease stability requirement of an oral drug among the Uricase tested.

TABLE 13
Stability against protease trypsin

| TRYPSIN | 0 H | 0.5 H | 1.0 H | 1.5 H | 2.0 H | 3.0 H | 4.5 H |
|---|---|---|---|---|---|---|---|
| Amano | 22.38 | 22.22 | 21.95 | 21.27 | 22.34 | 21.66 | 21.16 |
| Biozyme | 11.05 | 11.39 | 11.52 | 11.58 | 11.71 | 11.95 | 11.17 |
| In-House | 22.73 | 22.50 | 23.21 | 23.54 | 23.86 | 25.50 | 25.37 |
| Trypsin | 0 H | 0.5 H | 1.0 H | 1.5 H | 2.0 H | 3.0 H | 4.5 H |
| Amano | 100% | 99% | 98% | 95% | 100% | 97% | 95% |
| Biozyme | 100% | 103% | 104% | 105% | 106% | 108% | 101% |
| In-House | 100% | 99% | 102% | 104% | 105% | 112% | 112% |

Table 13 describes the stability of Amano, Biozyme and In-house Uricases against trypsin. The top table has the actual U/mg at a given time and the bottom table expresses that as % activity from the 0 hour time point. All of them are stable for 4.5 hours at 37° C. when 1 mg/ml of Uricases were mixed with 1 mg/ml trypsin in a 50 parts Uricase to 1 part protease ratio.

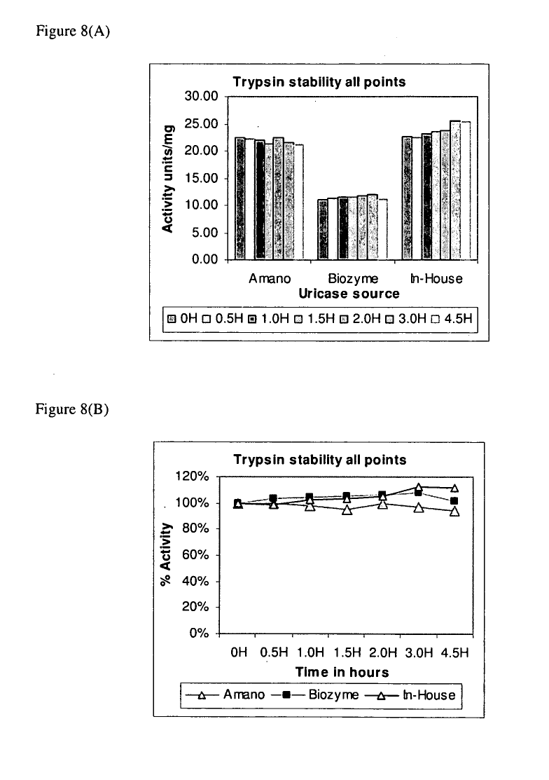

FIG. 8(A) shows stability against trypsin at different time points as Units/mg FIG. 8(B) shows activity in terms of % remaining activity compared to 0 hour activity. All Uricases tested (Amano, Biozyme and In-house Uricase) are stable for 4.5 hours in the presence of trypsin.

TABLE 14
Stability against protease chymotrypsin

| | AMANO | BIOZYME | | IN HOUSE | | GENZYME |
|---|---|---|---|---|---|---|
| TIME | U/mg | U/mg | TIME | U/mg | TIME | U/mg |
| 0.00 | 22.64 | 11.26 | 0.00 | 21.84 | 0.00 | 12.15 |
| 0.50 | 18.41 | 2.56 | 0.50 | 14.85 | 0.20 | 0.00 |
| 1.00 | 15.93 | 0.99 | 1.00 | 13.51 | 0.43 | 0.00 |
| 1.50 | 14.79 | 0.42 | 1.50 | 11.82 | 0.50 | 0.00 |
| 2.00 | 14.58 | 0.24 | 2.00 | 10.50 | | |
| 3.00 | 9.45 | 0.05 | 3.00 | 9.26 | | |
| 4.50 | 7.71 | 0.06 | 4.00 | 8.19 | | |
| TIME | AMANO | BIOZYME | TIME | IN HOUSE | TIME | GENZYME |
| 0.00 | 100% | 100% | 0.00 | 100% | 0.00 | 100% |
| 0.50 | 81% | 23% | 0.50 | 68% | 0.20 | 0% |
| 1.00 | 70% | 9% | 1.00 | 62% | 0.43 | 0% |
| 1.50 | 65% | 4% | 1.50 | 54% | 0.50 | 0% |
| 2.00 | 64% | 2% | 2.00 | 48% | | |
| 3.00 | 42% | 0% | 3.00 | 42% | | |
| 4.50 | 34% | 0% | 4.00 | 37% | | |

Table 14 describes the stability of Amano, Biozyme and In-house Uricases against chymotrypsin. Amano and In-house Uricase are more stable compared to Biozyme and Genzyme. Top table has actual U/mg at a given time and bottom table expresses that as % activity from 0 hour time point.

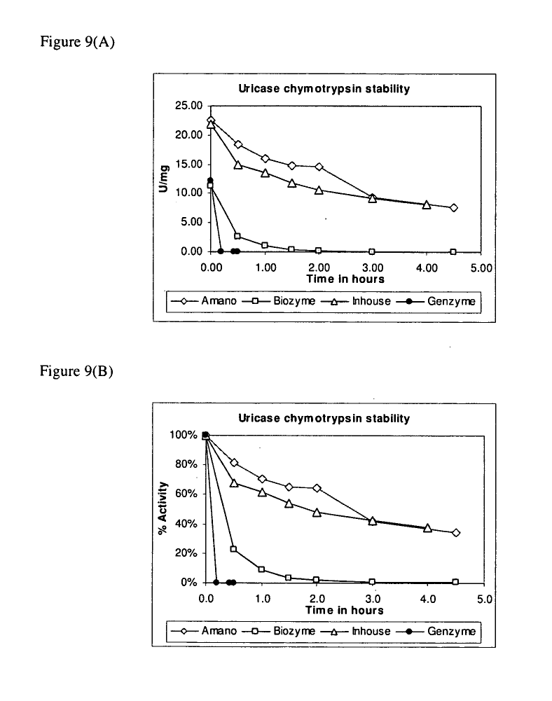

FIG. 9(A) shows stability against chymotrypsin at different time points as Units/mg. FIG. 9(B) shows activity in terms of % remaining activity compared the to 0 hour activity. Amano and In-house Uricases retained about 37% activity after 4 hour incubation compared to the 0 hour time point. Biozyme and Genzyme Uricase are more susceptible to chymotrypsin. Biozyme lost most of the activity within an hour of incubation and Genzyme lost all activity within 12 minutes.

### Conclusions

Characterization of Uricases suggests that Amano Uricase and In-house Uricase have similar characteristics and are preferred candidates as an oral drug. None of the tested Uricases are stable at lower pHs but they can be formulated for pH stability. Activity pH profile can not be changed without changing molecule and when considered this property, Amano and In-house Uricase have advantages over the other Uricases. Biozyme Uricase has similar activity pH profile but activity units are lower compared to Amano and In-house Uricase. Beneficial properties of each enzyme can be summarized as in Table 15 below.

TABLE 15
Summary of characterization

| URICASE | | | GENZYME/ | | IN- |
|---|---|---|---|---|---|
| PROPERTY | AMANO | BIOZYME | FLUKA | T-129 | HOUSE |
| Purity | X | | | X | X |
| pI | | | | X | |
| Activity | X | X | | | X |
| pH profile | | | | | |
| pH stability | | | | | |
| Protease | X | | | | X |
| stability | | | | | |

An X marks the beneficial properties of each enzyme for oral delivery.

### Example 16

### Crystallization of Uricase

### Principle

Hyperuricemia is elevated uric acid levels in the blood which can predispose for gout, hypertension and kidney stones. Causes of hyperuricemia can be primary (increased uric acid levels due to purine metabolism), and secondary (high uric acid levels due to another disease or condition). Consumption of a purine-rich diet (high protein and fat and beer) is one of the main causes of hyperuricemia in Western World. Lowering of the concentration of uric acid in plasma and urine is an important part of medical treatment, because this reduction changes the supersaturation index and lowers/prevents formation of monosodium urate crystals. Present therapy, such as an allopurinol treatment is limited in its effectiveness, and there is a need for a novel treatment. Thus, we are developing a novel approach, an oral therapy with modified crystalline uricase that should be stable and active along the gastrointestinal tract. This can complement or replace existing therapies.

In the intestine, uricase will work by breaking down uric acid, creating a concentration gradient between the bloodstream/kidney and intestinal lumen. This gradient will give rise for more uric acid being eliminated through the GI tract, thereby reducing uric acid levels in the bloodstream. Also, uricase can be formulated as insoluble and not absorbable enzyme thereby preventing any immunogenicity against drug.

### Equipment and Materials

### Equipment

Desalting column, Econo-Pac, 10DG disposable chromatography columns
UV Spectrometer, Agilent 8453
pH meter, Accumet Basic Ab15
Microscope, Olympus BX60
Centrifuge, Eppendorf 5415C
Tumbler, Fisher Scientific, Hematology/Chemistry mixer
Bench top centrifuge, Beckman GS-6R

### Reagents

Lyophilized Biozyme uricase, Cat # U5
HEPES, Sigma, Cat #: H4034-1G
MgCl₂, Sigma, Cat # M2670-500G
PEG 8000, Sigma, Cat # P-4463-1 Kg
Filtered deionized water

### Procedure

### Buffer Preparation

1 M HEPES, pH 7.50: Dissolve 238 g in 750 ml deionized water. Adjust pH to 7.5 and make up to 1 L volume. Re-check pH and adjust if necessary.

50% PEG 8000 (w/v): To prepare 200 mL of 50% of PEG 8000, 100 g PEG 8000 is weighed out and added to a beaker containing ˜120 mLl of stirring deionized water. The PEG can be added all together. Note: Adding the PEG to stirring water helps dissolve the PEG quicker. When the PEG is mixed, but not dissolved, pour the mixture into a graduated cylinder and add water up to −170 mL and re-transfer back to beaker and stir until dissolved. The graduated cylinder is re-used for measuring to 200 mL and rinsed later with the completely dissolved 50% PEG solution. To dissolve PEG 8000, it has to be ≦50% saturation.

1 M Magnesium chloride: Dissolve 20.3 g in 70 ml of dH₂0. Once dissolved, make up to 100 mL

Crystallization buffer: Add 10 mL of 1 M HEPES, pH 7.50, 36 ml of 50% PEG 8000, 16 mL of 1 M Magnesium chloride and 38 mL of dH₂O. Filtered through 0.22 μm filter.

### Screening and Optimization:

In one protocol, Biozyme uricase was first desalted in water at a concentration of 50 mg/mL by A₂₈₀ₙₘ. Extinction coefficient for Biozyme uricase is considered=1.

The crystallization reagent consisted of:

0.1 M TrisCl pH 8.5, 30% PEG 8K, 0.25 M MgCl₂.

The protein: reagent ratio was 1:1. After mixing, the batch was incubated overnight at RT without tumbling. The final volume was 1 mL.

The above condition was adjusted for further scale up and good yield. Therefore, optimization involved alternating the concentrations of MgCl₂, protein (enzyme+/−desalting), PEG 8K and also modifying final pH.

### Batch Crystallization Protocol

To crystallize 10 g of Biozyme uricase, 5 mL batches were made using the optimal crystallization condition observed from crystallization screening process.

a) First 450 mg of uricase was weighed and dissolved in 5 mL of water or 4.5 g was weighed out and dissolved in 50 mL water. This gave a protein concentration of 41 mg/mL by A₂₈₀ₙₘ.

b) Then the uricase solution was filtered thru 0.22 μm filter.

c) Crystallization reagent was made as

0.1 M HEPES, pH 7.50

160 mM MgCl₂

18% PEG 8K

d) Crystallization was set-up using a protein: reagent ration of 1:1.

2.5 mL of crystallizing reagent was added to 2.5 mL of protein. Once the reagent was added, the solution was immediately mixed by pipetting up and down using a 5 mL pipetter.

e) The mixture was incubated overnight at RT with tumbling.

f) After crystallization, the crystals were washed at half the volume, to ensure the crystals would not dissolve. 5 mL crystal batches were centrifuged at 1500 rpm for 10 mM, supernatant pipetted off, and mother liquor added to 2.5 mL. The mother liquor consisted of 0.05 M HEPES, pH 7.50, 80 mM MgCl₂, 9% PEG 8K.

### Results

Crystals were obtained using a 1:1 ratio of 0.1 M HEPES, pH 7.50, 0.16 M MgCl₂, 18% PEG 8K mixed with ˜41 mg/mL Biozyme uricase as determined by A₂₈₀ₙₘ. The final protein concentration after addition of crystallizing reagent was 20 mg/mL. Lyophilized Biozyme uricase contains 50% excipients, but we found that Biozyme uricase can be successfully crystallized without desalting. In addition, crystallization was instant once crystallization reagent was added to the enzyme.

Uricase crystals are soft and a bridge was made with cover slips for viewing of the crystals under a microscope. The size and shape of the crystals varied with protein concentration. When the protein concentration is higher such as 30 mg/mL, the crystal size is larger and most have a rice shape. The crystal yield is 73-77% at the 5 mL scale. Crystallization at a scale >5 mL was not carried as extra material for scale-up trials was not available from Biozyme.

### Conclusions

10 g of Biozyme uricase was crystallized successfully by setting up multiple batches at a 100 mg scale. The crystal yield was 73-78%.

### Example 17

### Crystallization of Uricases

### Objective

This report is a summary of crystallization trials for Uricases. Uricases from different sources were screened to find the condition for crystallization. They were selected for this purpose based on availability or to fulfill a requirement for in-vivo studies. Amano, Biozyme, Genzyme, T-129 Uricase and In-House Uricase were crystallized in batches of different scales.

### Equipment and Material

### Equipment

Microscope with camera: Olympus optical co., LTD, Model # BX51TF

Magnetic Stir plate: Fisher, Cat # 550442ST

UV Spectrophotometer: Agilent, Model # 8453

pH meter: Fisher, Model Accumet Basic Ab15

Centrifuge: Eppendorf Model 5415C

Amicon® ultra-4 Centrifugal filter unit: Millipore, Cat # UFC801024

Amicon ultra-15 Centrifugal filter unit: Millipore, Cat # UFC901024

Vortex mixer: Fisher, Cat # 02215370

Balance: Denver instrument Company, Model # A-200DS

Hematology/Chemistry mixer: Fisher, Cat # 14-059-346

### Material

VDX Crystallization plates: Hampton Research, Cat # HR3-140

22 mm×0.22 mm Siliconized circle cover slides: Hampton Research, Cat # HR3-233

Forceps: Fisher, Cat # 08-906

Immersion oil Type B: Hampton Research, Cat # HR3-615

Microscope slides: Fisher, Cat # 12-550A

Microscope cover glass: Fisher, Cat # 12-548-A

UV transparent disposable cuvettes semi-micro: Fisher, Cat # 13-688-73

Econo-Pac, 10DG desalting column: Bio-RAD, Cat # 732-2010

7M Sodium Formate: Hampton research, Cat # HR2-547

Tris Base: Fisher, Catalog #BP152-5

HEPES: Sigma, Cat # H4034-1KG

Boric Acid Sigma, Cat # B0394-500G

MES: Sigma, Cat # M2933-1KG

1N Hydrochloric acid (1N HCl): Fisher, Catalog #SA48-500

1N Sodium hydroxide (1N NaOH): Fisher, Cat # AC12426-0010

Sodium hydroxide pellets: Fisher, Cat # AC42433-5000

Sodium chloride (NaCl): Fisher, Cat # 5640-500G

Sodium citrate (Na citrate): Fisher, Cat # S279-10

Magnesium chloride (MgCl2): Sigma, Cat # M2670-500G

Sodium acetate (Na acetate): Fisher, Cat # S607-212

50% Polyethylene glycol 2000 Monomethyl ether (MPEG2K): Fisher, Cat # NC9419179

Polyethylene glycol 4000 (PEG 4K): Fluka, Cat # 81240

Polyethylene glycol 8000 (PEG 8K): Fluka, Cat # 81268

Polyethylene glycol 20000 (PEG 20K): Fluka, Cat # 81300

DL Dithiothreitol (DTT): Sigma, Catalog #D0632-100G

2-Propanol (IPA): Fisher, Cat # A451-4

Ethanol: Sigma, Cat # 459844-4L

Crystallization kits

JBS1 to 10: Jena biosciences, Cat # CS-101L, CS-102L, CS-103L, CS-104L, CS-105L, CS-106L, CS-107L, CS-108L, CS-109L, CS-110L

Crystal screen, Crystal screen II, MPD Grid screen: Hampton Research, Cat # HR2-110, HR2-112, HR2-215

Wizard I, Wizard II, Cryo I, Cryo II, Ozma™ PEG-Ion 4K: Emerald biosystems, Cat # EBS-WIZ-1, EBS-WIZ-2, EBS-CRYO-1, EBS-CRYO-2, EBS-PEG-4

Stura footprint screens: Molecular dimensions, Cat # MD1-20

### Uricases

Amano Uricase: Amano, Cat # UR-2, Source Candida utilis

Biozyme Uricase: Biozyme, Cat # U5, Source Recombinant E. coli expressed enzyme Original cDNA from Candida species

Genzyme Uricase: Genzyme, Cat # 1701, Source Bacillus fastidiosus

Uricase T-129: Genzyme, Cat # T-129, Source Arthrobacter globiformis

In-house Uricase: Altus Uricase, Source Recombinant E. coli expressed enzyme Original cDNA from Candida utilis

### Hanging Drop Screening

### Procedure

24 well hanging drop screening VDX plates were labeled (Bottom and cover) appropriately with Plate number, protein and screening kit name.

A syringe was filled with Immersion oil type B and a pipette tip (General purpose tip for 10-200 μl from fisher) was attached to it. Using this syringe, oil was applied to the well lips.

600 μl of pre made crystallization screening reagents from kits were taken in to appropriate wells.

On a circular siliconized glass cover slide, 3 μl of reagent was taken from well. Mixed with 3 μl of protein (Uricase) in different random ratios and several drops were made.

Inverted cover slide carefully and put it on top of the well while making sure it is properly sealed.

Completed entire plate and covered it. Incubated plates overnight at room temperature.

Observed and recorded results for the drops using a microscope attached to a camera for taking pictures.

### Screening Plan Summary

Screening kits and proteins used for screening are summarized in Table 16 below.

TABLE 16
Summary of hanging drop screening

| URICASE | PREPARATION | SCREENING KITS | NO. OF HITS |
|---|---|---|---|
| Genzyme | Desalted in 10 mM Tris 7.5 | Wizard I | |
| | | JBS 3, 4, 5, 7, 8, 9, 10 | 15 |
| | | Stura footprint (1-24) | 2 |
| Total hits | | | 17 |
| Amano | Desalted in 10 mM Tris pH 7.5 | JBS 1-5, 8, 9 | 11 |
| | | Wizard I, Wizard II, Cryo I, Cryo II | 2 |
| | Original in 10 mM Tris pH 7.5 | JBS 1-10 | 6 |
| | | MPD Grid screen | 4 |
| | Desalted in 10 mM Tris pH 8.5 | Wizard I, Wizard II, Cryo I, Cryo II | 1 |
| | | Crystal screen, Crystal screen II | 0 |
| | | Ozma 4K | 0 |
| | Desalted in DI Water | JBS 1 to 8 | 3 |
| | | Stura footprint | 0 |
| | Original in DI Water | Crystal screen, Crystal screen II | 2 |
| | Desalted in HEPES 7.5, 50 mM NaCl | JBS 1-5, 7, 8 | 4 |
| | All different preparations | Home made reagents | 23 |
| Total hits | | | 56 |
| Biozyme | Desalted in DI Water | Crystal screen, Crystal Screen II | 12 |
| | | Wizard I, Wizard II, Cryo I, Cryo II | 9 |
| | | Ozma 4K | 26 |
| | | JBS 9, 10 | |
| Total hits | | | 47 |
| T-129 | Original in 10 mM Tris 7.5 | JBS 1, 2 | 8 |
| | | JBS 3, 4, 5, 7, 8 | 22 |
| | | Crystal Screen | 13 |
| Total hits | | | 43 |
| In-House* | Desalted in 20 mM Tris pH 7.5 | Wizard I, Wizard II, Cryo I, Cryo II | 1 |
| | | JBS 1, 2, 3, 4, 8, 9 | 4 |
| Total Hits | | | 5 |

### Batch Optimization

After identifying hits from hanging drop screening, the most promising conditions were duplicated to get crystals in batches. Conditions were optimized to get crystals and to get good yield and activity after crystallization. Protein concentrations, different reagent component concentrations and pH combinations, as well as different temperature incubations were tried for optimization. Best conditions found for crystallization are listed in Table 17 below with their yield, activity and scale of crystallization.

TABLE 17
Batch optimization summary

| | | PROTEIN | | | | ACTIVITY | | |
|---|---|---|---|---|---|---|---|---|
| URICASE | REAGENT | CONC. | | RATIO | YIELD | U/mg | SCALE | |
| Genzyme | 0.8M Na Formate, | 25 | mg/ml | 1 protein + | 45-60% | 20-25 | 2 | ml |
| | 15% PEG 4000, pH | | | 6 Reagent | | (pH 9.1) | | |
| | 6.5 | | | | | | | |
| Amano | 20% PEG 4000, | 50 | mg/ml | 1 protein + | ~90% Crystals | 20-25 | 50 | μl |
| | 0.1M Na citrate, | | | 4 Reagent | with | | | |
| | 20% IPA | | | | precipitate) | | | |
| Biozyme | 30% PEG 8000, | 15 | mg/ml | 1 protein + | 70% | 25 | 20 | μl |
| | 0.1M Tris 8.5, 0.25M | | | 1 Reagent | | | | |
| | MgCl₂ | | | | | | | |
| Genzyme | 19% PEG 20K, | 58-62 | mg/ml | 1 protein + | 75% | 19 | 1 | ml |
| T-129 | 0.1M Borate 8.5, | | | 1 Reagent | | | | |
| | 0.1M MgCl₂ | | | | | | | |
| In-House | 20% PEG 4000, | 50 | mg/ml | 1 protein + | 80-90% | 25 | 0.3 | ml |
| | 0.1M Na citrate, | | | 2 Reagent | | | | |
| | 20% IPA | | | | | | | |

### Genzyme Uricase

Genzyme Uricase was crystallized. Optimization trials were started but not continued as the pH profile of this enzyme was not suitable for an oral drug. The crystallization procedure mentioned in this report is the original condition without further optimization and was used only to produce crystals for characterization purposes.

### Protein Preparation

10 mM Tris buffer pH 7.5: 60.57 mg of Tris base was dissolved in −30 ml of DI Water. pH was adjusted to 7.5 with 1 N HCl and final volume was made up to 50 ml with DI Water. pH was checked again and adjusted to 7.5 if needed (pH reading was without stirring the solution while reading).
Genzyme Uricase powder was dissolved in 10 mM Tris buffer pH 7.5.
Uricase was desalted in 10 mM Tris buffer pH 7.5 using a DG-10 desalting column from Bio-RAD.
Protein concentration was adjusted to 25 mg/ml by A₂₈₀ considering ε of 1 for A₂₈₀ reading after desalting.
   Reagent preparation (15% PEG 4000, 0.8 M Na Formate):
50% w/v PEG 4000 stock: 10 grams of PEG 4000 was dissolved in DI Water to make total volume of 20 ml.
7 M Na Formate: 7 M Na Formate stock was purchased from Hampton research
Crystallization reagent Preparation (10 ml): 3 ml of 50% PEG 4000, 1.143 ml of 7 M Na Formate and 5 ml of DI Water were mixed together. pH was tested and adjusted to 6.5 using measures volume of 10 N HCl. Added DI Water to make final volume to 10 ml.

### Batch Preparation

200 μl of Genzyme Uricase at 25 mg/ml was taken in a tube.
1200 μl of crystallization reagent was added drop wise to the protein while mixing gently by shaking the tube.
Incubated tube at room temperature (23-25° C.) overnight.
Batches were observed the next day for crystals.

### Amano Uricase

### Protein Preparation

10 mM Tris buffer pH 7.5: 60.57 mg of Tris base was dissolved in ˜30 ml of DI Water. pH was adjusted to 7.5 with 1 N HCl and final volume was made up to 50 ml with DI Water. pH was checked again and adjusted to 7.5 if needed (pH reading was without stirring the solution while reading).
100 mM DTT: 154.3 mg of DTT was dissolved in DI Water to make a total volume of 10 ml. Made aliquots and stored at −20° C.
Amano Uricase powder was dissolved in 10 mM Tris pH 7.5, 2 mM DTT buffer (1 ml of 100 mM DTT was added to 50 ml of 10 mM Tris pH 7.5 just before use).
Desalted Uricase in 10 mM Tris pH 7.5, 2 mM DTT buffer using a DG-10 desalting column from Bio-RAD.
Protein concentration was adjusted to 50 mg/ml by A₂₈₀ considering ε of 1 for A₂₈₀ reading after desalting.
   Reagent Preparation (20% PEG 4000, 0.1 M Na citrate, 20% IPA; 10 ml):
Weighed 2 g of PEG 4000, 2 g of IPA and 294.1 mg of Na citrate dehydrate and added in a 15 ml tube.
Dissolved with minimum amount of DI Water and then volume was made up to 10 ml with DI Water and stored at room temperature.

### Batch Preparation

Took 10 μl of desalted Amano Uricase in a 0.5 ml eppendorf tube.
Added 40 μl of crystallization reagent and mixed gently by tapping the tube with finger.
Tube was then incubated at room temperature (23-25° C.) overnight.
Batches were observed the next day for crystals.

### Biozyme Uricase

### Protein Preparation

Biozyme Uricase powder was dissolved in DI Water and desalted in DI Water using DG-10 desalting columns from Bio-RAD.
Protein concentration was adjusted to 15 mg/ml by A₂₈₀ considering ε of 1 for A₂₈₀ reading after desalting.
   Reagent preparation (30% PEG 8000, 0.1 M Tris 8.5, 0.25 M MgCl2):
50% w/v PEG 8000 stock: 10 grams of PEG 8000 was dissolved in DI Water to make total volume 20 ml.
1 M Tris pH 8.5 buffer stock: 6.057 grams of Tris base was dissolved in ˜30 ml of DI Water. pH was adjusted to 8.5 with 1 N HCl and the final volume was made up to 50 ml with DI Water. pH was checked again and adjusted to 8.5 if needed (pH reading was without stirring the solution while reading).
1 M MgCl₂ stock: 2.03 g of MgCl₂ was dissolved in DI Water to make a total volume of 10 ml.
Crystallization reagent preparation (100 μl): 60 μl of 50% PEG 8000, 10 μl of 1 M Tris pH 8.5, 25 μl of 1 M MgCl₂ and 5 μl of DI Water were mixed well to make 100 μl of crystallization reagent.

### Batch Preparation

Took 10 μl of desalted Biozyme Uricase in a 0.5 ml eppendorf tube.
Added 10 μl of crystallization reagent and mixed gently by tapping the tube with finger.
Tube was then incubated at room temperature (23-25° C.) overnight.
Batches were observed the next day for crystals.

### T-129 Uricase

### Protein Preparation

10 mM Tris buffer pH 7.5: 60.57 mg of Tris base was dissolved in −30 ml of DI Water. pH was adjusted to 7.5 with 1 N HCl and the final volume was made up to 50 ml with DI Water. pH was checked again and adjusted to 7.5 if needed (pH reading was without stirring the solution while reading).
T-129 Uricase was dissolved in 10 mM Tris pH 7.5. Protein concentration was checked by A₂₈₀ reading and adjusted to ˜60 mg/ml (Acceptable range is 58-62 mg/ml) considering c of 1 for A₂₈₀ reading.

### Reagent Preparation

50% w/v PEG 20000 stock: 10 grams of PEG 20000 was dissolved in DI Water and the total volume was adjusted to 20 ml.
0.5 M Borate buffer pH 8.5 stock: 1.55 g of boric acid was dissolved in 40 ml of DI Water. pH was adjusted to 8.5 using 4 N NaOH. Final volume was made up to 50 ml. pH was checked again and adjusted to 8.5 if needed.
1 M MgCl₂ stock: 2.03 g of MgCl₂ was dissolved in DI Water to make total volume of 10 ml.
Crystallization reagent preparation (1 ml): 380 μl of 50% PEG 20000, 200 μl of 0.5 M Borate buffer pH 8.5, 100 μl of 1 M MgCl₂ and 320 μl of DI Water were mixed nicely to make 1 ml of crystallization reagent

### Batch Preparation

Took 0.5 ml of T-129 in 10 mM Tris 7.5 at ˜60 mg/ml concentration (58 to 62 mg/ml) in a small glass vial with magnet for stirring.
While mixing on a stir plate at a speed of 7, added 0.5 ml of crystallization reagent drop by drop. Protein and reagent were mixed for about 1 minute after addition of reagent was complete.
Stopped stirring and incubated batch at room temperature (23-25° C.) for 15 hours. (If incubated longer than 16 hours needle shaped crystals turn in to plates).

### In-House Uricase

### Protein Preparation

100 mM DTT: 154.3 mg of DTT was dissolved in DI Water to make total volume 10 ml. Made aliquots and stored at −20° C.
In-House Uricase was purified by purification team (Report # 0006962) and supplied in 20 mM Tris pH 7.5 buffer.
DTT was added to this protein to a final concentration of 1 mM DTT. Protein concentration was adjusted to 50 mg/ml by A₂₈₀ considering c of 1 for A₂₈₀ reading.
   Reagent preparation (20% PEG 4000, 0.1 M Na citrate, 20% IPA; 10 ml):
Weighed 2 g of PEG 4K, 2 g of IPA and 294.1 mg of Na citrate dehydrate and added in a 15 ml tube.
Dissolved with minimum amount of DI Water and then volume was made up to 10 ml with DI Water and stored at room temperature.

### Batch Preparation

Took 100 μl of 1n-house Uricase at 50 mg/ml concentration in a 0.5 ml eppendorf tube.
Added 200 μl of crystallization reagent and mixed gently by tapping the tube with finger or by inverting the tube few times.
Tube was then incubated at room temperature (23-25° C.) overnight.
Batches were observed the next day for crystals.

### Results

### Genzyme Uricase

Genzyme Uricase was crystallized in batches using this condition:
Reagent: 15% w/v PEG 4000, 0.8 M Na Formate pH 6.5
Protein: 25 mg/ml Genzyme Uricase desalted in 10 mM Tris pH 7.5
Ratio: 1 protein+6 Reagent

Crystals were very sensitive to centrifugation or other similar mechanical stresses like vortexing as they tend to stick together. So it was difficult to formulate crystals and to make a homogenous suspension. Some hanging drop screening was performed to find another condition for crystallization but then Amano crystallization was considered and screening was stopped.

### Amano Uricase

### Hanging Drop Screening

Many hits were found in hanging drop screening for Amano. Hits were mainly observed with polyethylene glycol (PEG) and organics. Results for hanging drop screening were recorded in an excel sheet. Pictures were taken for promising conditions and crystals and saved as a .jpg images. Pictures were inserted in a word document with the conditions listed under the picture. Some promising hanging drop conditions are listed in Table 18 below.

TABLE 18
Hanging drop hits for Amano Uricase

| | | CONCENTRATION | | KIT | |
|---|---|---|---|---|---|
| HIT # | PROTEIN TYPE | BY A₂₈₀ | | REAGENT # | REAGENT COMPOSITION |
| 1 | Desalted in DI | 9.9 | mg/ml | JBS 8 C2 | 12% Ethanol, 0.1M acetate |
| | Water | | | | 4.6, 4% PEG 400 |
| 2 | Desalted in 10 mM | 10 | mg/ml | JBS 2 B6 | 20% PEG 4K, 0.1M Tris 8.5, |
| | Tris 7.5 | | | | 0.2M CaCl2 |
| 3 | Desalted in 10 mM | 10 | mg/ml | JBS 3 C3 | 20% PEG 4K, 0.1M HEPES |
| | Tris 7.5 | | | | 7.5, 10% IPA |
| 4 | Desalted in 10 mM | 10 | mg/ml | JBS 5 D1 | 18% PEG 10K, 0.1M Tris 8.5, |
| | Tris 7.5 | | | | 0.1M NaCl, 20% Glycerol |
| 5 | Original in 10 mM | 5 | mg/ml | JBS 8 A5 | 60% MPD, 0.1M Acetate 4.6, |
| | Tris 7.5 | | | | 0.01M CaCl2 |
| 6 | Original in 10 mM | 5 | mg/ml | JBS 8 A6 | 60% MPD, 0.02M Na acetate |
| | Tris 7.5 | | | | |
| 7 | Original in 10 mM | 5 | mg/ml | JBS 8 D4 | 60% Ethanol, 0.05M Na |
| | Tris 7.5 | | | | acetate, 1.5% PEG 6K |
| 8 | Original in 10 mM | 10 | mg/ml | MPD grid | 65% MPD, 0.1M citric acid |
| | Tris 7.5 | | | D1 | pH 4.0 |
| 9 | Original in 10 mM | 10 | mg/ml | MPD grid | 65% MPD, 0.1M Tris 8.0 |
| | Tris 7.5 | | | D5 | |
| 10 | Desalted in 10 mM | 18 | mg/ml | JBS 1 D3 | 25% PEG 2000 MME |
| | Tris 7.5, 2 mM DTT | | | | |
| 11 | Desalted in 10 mM | 18 | mg/ml | JBS 3 A2 | 10% PEG 4K, 20% IPA |
| | Tris 7.5, 2 mM DTT | | | | |
| 12 | Desalted in 10 mM | 18 | mg/ml | JBS 3 B2 | 15% PEG 4K, 0.1M Na |
| | Tris 7.5, 2 mM DTT | | | | citrate 5.6, 0.2M (NH₄)₂SO₄, |
| 13 | Desalted in 10 mM | 18 | mg/ml | JBS 3 C1 | 20% PEG 4K, 20% IPA, 0.1M |
| | Tris 7.5, 2 mM DTT | | | | Na citrate |

### Batch Crystallization

Batch crystallization for Amano Uricase was tried for many promising hanging drop conditions. All conditions gave crystals with precipitates. Some conditions are listed in Table 19.

TABLE 19
Amano Uricase crystals in batches

| | CONCENTRATION | REAGENT | |
|---|---|---|---|
| PROTEIN TYPE | BY A₂₈₀ | COMPOSITION | RATIO |
| Original in 10 mM | 29.5 mg/ml | 90% Ethanol, | 1 protein + 1 |
| Tris 7.5, 2 mM DTT | | 0.1M NaCl | reagent |
| Desalted in 10 mM | 49 mg/ml | 20% PEG 2K | 1 protein + 2 |
| Tris 7.5, 2 mM DTT | | MME, 0.1M MES | reagent |
| | | 6.5, 0.1M Na | |
| | | acetate | |
| Desalted in 10 mM | 49 mg/ml | 10% PEG 4K, 20% | 1 protein + 2 |
| Tris 7.5, 2 mM DTT | | IPA | reagent |
| Desalted in 10 mM | 49 mg/ml | 10% PEG 4K, 20% | 1 protein + 3 |
| Tris 7.5, 2 mM DTT | | IPA | reagent |

Best condition for crystallization was
Reagent: 20% w/v PEG 4000, 20% w/v IPA, 0.1 M Na citrate
Protein: 50 mg/ml Amano Uricase desalted in 10 mM Tris pH 7.5, 2 mM DTT
Ratio: 1 protein+2 Reagent

The resulting crystals were cube shaped crystals with precipitates.

### Biozyme Uricase

### Hanging Drop Screening

Hanging drop screening for Biozyme was performed with desalted Biozyme. Many hits were observed in hanging drops mainly with polyethylene glycol (PEG) of different molecular weight. Crystals were mainly flat plate shaped except for one reagent Wizard II 43 which gave hexagonal shaped crystals. Some promising hanging drop conditions are listed in Table 20 below.

TABLE 20
Hanging drop hits for Biozyme Uricase

| | | CONCENTRATION | KIT | |
|---|---|---|---|---|
| HIT # | PROTEIN TYPE | BY A₂₈₀ | REAGENT # | REAGENT COMPOSITION |
| 1 | Desalted in DI | 15 mg/ml | CS 6 | 30% PEG 4K, |
| | Water | | | 0.1M Tris 8.5, |
| | | | | 0.2M MgCl₂ |
| 2 | Desalted in DI | 15 mg/ml | Wizard I 21 | 20% PEG 8K, |
| | Water | | | 0.1M HEPES 7.5 |
| 3 | Desalted in DI | 15 mg/ml | Wizard II 3 | 20% PEG 8K, |
| | Water | | | 0.1M Tris 8.5, |
| | | | | 0.2M MgCl₂ |
| 4 | Desalted in DI | 15 mg/ml | Wizard II 43 | 10% PEG 8K, |
| | Water | | | 0.1M Tris 7.0, |
| | | | | 0.2M MgCl₂ |
| 5 | Desalted in DI | 15 mg/ml | Ozma 4K 39 | 20% PEG 4K, |
| | Water | | | 0.2M Na formate |

### Batch Crystallization

Batch crystallization for Biozyme Uricase was tried with the hanging drop hit with Wizard II reagent 3. The condition was 20% PEG 8K, 0.1 M Tris 8.5, 0.2 M MgCl₂. Optimization trials were set up with different concentrations of PEG 8K and MgCl₂. Different reagent to protein ratios, different protein concentration, buffers and pHs were also tried for batch optimization.

Best condition for crystallization was
Reagent: 30% PEG 8K, 0.1 M Tris 8.5, 0.25 M MgCl₂
Protein: 15 mg/ml Biozyme Uricase desalted in DI Water
Ratio: 1 protein+1 Reagent

### T-129 Uricase

### Hanging Drop Screening

Hanging drop screening was performed with Uricase in 10 mM Tris buffer pH 7.5 at different protein concentrations. Many conditions were identified for crystallization in hanging drop setting. PEG and salt combination worked best for T-129 crystallization. Some promising hanging drop conditions are listed in Table 21 below.

TABLE 21
Hanging drop hits for T-129 Uricase

| | | CONCENTRATION | | KIT | |
|---|---|---|---|---|---|
| HIT # | PROTEIN TYPE | BY A₂₈₀ | | REAGENT # | REAGENT COMPOSITION |
| 1 | Original in | 8 | mg/ml | JBS 1 | 20% MPEG 2K, |
| | 10 mM Tris | | | D4 | 0.1M MES 6.5, |
| | 7.5 | | | | 0.1M Na acetate |
| 2 | Original in | 8 | mg/ml | JBS 1 | 30% PEG 3K, 0.1M |
| | 10 mM Tris | | | D6 | Tris 8.5, 0.2M |
| | 7.5 | | | | Li₂SO₄ |
| 3 | Original in | 8 | mg/ml | JBS 2 | 22% PEG 4K, 0.1M |
| | 10 mM Tris | | | C1 | HEPES 7.5, 0.1M |
| | 7.5 | | | | Na acetate |
| 4 | Original in | 24 | mg/ml | JBS 3 | 25% PEG 4K, 0.1M |
| | 10 mM Tris | | | C6 | Na citrate 5.6, 0.2M |
| | 7.5 | | | | (NH₄)₂SO₄ |
| 5 | Original in | 24 | mg/ml | JBS 5 | 17% PEG 20K, |
| | 10 mM Tris | | | D5 | 0.1M Tris 8.5, |
| | 7.5 | | | | 0.1M MgCl₂ |
| 6 | Original in | 24 | mg/ml | JBS 5 | 18% PEG 10K, |
| | 10 mM Tris | | | D1 | 0.1M Tris 8.5, |
| | 7.5 | | | | 0.1M NaCl, 20% |
| | | | | | Glycerol |

### Batch Crystallization

Batch crystallization for T-129 Uricase was tried with a few conditions from JBS 1, 2, 3, 4, and 5. Optimization trials were set up with different concentrations of reagent components as well as different reagent to protein ratios, protein concentration, buffers and pHs.

Best condition for crystallization was
Reagent: 19% PEG 20K, 0.1 M Borate 8.5, 0.1 M MgCl₂
Protein: 60 mg/ml T-129 Uricase in 10 mM Tris 7.5
Ratio: 1 protein+1 Reagent

The resulting crystals were needle shaped crystals.

### In-House Uricase

### Hanging Drop Screening

In-house Uricase was screened in hanging drop without adding DTT to the sample. Hits observed were mainly with PEG and organics. Fewer hits were observed compared to other Uricases but two of these conditions crystallized Uricase in batches so further screening was not necessary. Some promising hanging drop conditions are listed in Table 22 below.

TABLE 22
Hanging drop hits for In-house Uricase

| | | CONCENTRATION | KIT | |
|---|---|---|---|---|
| HIT # | PROTEIN TYPE | BY A₂₈₀ | REAGENT # | REAGENT COMPOSITION |
| 1 | Desalted in | 24.5 mg/ml | Cryo I 40 | 40% Ethanol, 0.1M |
| | 20 mM Tris | | | Phosphate-citrate 4.2, |
| | 7.5 | | | 5% PEG 1K |
| 2 | Desalted in | 24.5 mg/ml | JBS 3 C1 | 20% PEG 4K, 0.1M |
| | 20 mM Tris | | | Na citrate, 20% IPA |
| | 7.5 | | | |
| 3 | Desalted in | 24.5 mg/ml | JBS 8 A2 | 50% MPD, 0.05M |
| | 20 mM Tris | | | Na acetate, 0.05M |
| | 7.5 | | | NaCl, 20% IPA |
| 4 | Desalted in | 24.5 mg/ml | JBS 8 D1 | 30% Ethanol, 0.1M |
| | 20 mM Tris | | | Na acetate, 10% |
| | 7.5 | | | PEG 6K |
| 5 | Desalted in | 24.5 mg/ml | JBS 9 C4 | 25% t-Butanol, 0.1M |
| | 20 mM Tris | | | Tris 8.5, 0.1M CaCl₂ |
| | 7.5 | | | |

### Batch Crystallization

Batch crystallization for In-House Uricase was tried with a condition that was found by Sibyl and then further optimized. Optimization trials were set up with different concentrations of reagent component as well as different reagent to protein ratios, and protein concentration.

Best condition for crystallization was
Reagent: 20% w/v PEG 4K, 0.1 M Na citrate, 20% w/v IPA
Protein: 50 mg/ml In-house Uricase in 20 mM Tris 7.5, 1 mM DTT
Ratio: 1 protein+2 Reagent

### Conclusions

Uricases from different sources could be crystallized in batches. Uricase T-129, Biozyme and Genzyme Uricases were easy to crystallize and many hits were found in hanging drop screening. Amano Uricase was more difficult to crystallize and required more screening to find a condition that could be optimized for batch crystallization. Addition of DTT was necessary for Amano and In-house Uricase to crystallize in batches. Amano Uricase crystallized with precipitates.

### Example 18

### Polyelectrolyte Coating of Crystalline Uricase

### Summary

Crystallized Candida utilis uricase was formulated to enhance crystalline stability. The crystallized uricase was non-covalently coated with two polyelectrolyte layers consisting of poly(methylene-co-guanidine-HCL) (PMG) and polyacrylic acid (PAA) to achieve this desired stability. This formulation stabilizes the crystalline structure of the enzyme, allowing removal from the mother liquor without dissolution. Coating of the crystals stabilizes them without incurring an appreciable loss in specific activity. The formulated crystals were developed for an oral feeding study in UoxKO mice.

### Introduction

Uricase (urate oxidase) is an enzyme that catalyzes the conversion of uric acid to allantoin, the terminal reaction in purine catabolism for most mammals with the exception of higher apes and humans. A purine-rich diet and in some cases impaired kidney function can contribute to higher than normal levels of uric acid in plasma—hyperuricemia. Due to its limited solubility, uric acid in high concentrations can form crystals, which can build up in deposits called tophi, which cause painful gouty arthritis. We are interested in formulating uricase as an oral enzyme therapy for hyperuricemia and gout.

Uricase from microbial sources such as Candida has a specific activity of 10-30

U/mg, depending on the species. Direct IP injection of soluble uricase, as well as oral delivery of lyophilized uricase, can significantly reduce plasma uric acid levels in UoX mice. Our goal is to develop a stable crystalline uricase formulation for oral delivery.

Like most enzyme crystals, uricase crystals are stable only in narrow range of conditions. The optimal conditions comprise the crystallization buffer, or mother liquor, and favor crystal formation from soluble protein. The crystals are only stable in this buffer, and usually dissolve if the buffer conditions are altered. Since we want to specifically test the effect of the crystalline enzyme in an animal study, we must ensure that the enzyme remains crystalline once administered. This can be accomplished by coating the crystal with polyelectrolyte multilayers, which also tends to preserve specific activity, especially in the case of uricase.

Candida uricase, acquired from Biozyme, has a pI of ˜5.2, meaning that at neutral pH the enzyme has a net negative charge. When added, a positively charged polymer such as poly(methylene-co-guanidine HCL) (PMG), will adhere to the surface of the crystals through favorable ionic interactions. If enough positively charged polymer coats the crystal it will have a net positive surface charge, which can be used to attract a second polymer layer, this time carrying a negative charge. Additional layers can be applied one at a time by alternating between positively and negatively charged polymers, centrifuging and removing excess polymer between coats. The formulation described in this example incorporates two polymer coats (PMG and PAA) onto Biozyme uricase crystals through layer-by-layer polyelectrolyte coating.

By adhering polyelectrolye layers to the surface of uricase crystals, we are able to create stable shells, tightly encapsulating the crystallized enzyme within. This provides mechanical and electrostatic incentive for the enzyme molecules to remain intact in crystal form when transferred to non-mother liquor buffer or other solution conditions.

### Materials and Methods

### Reagents

Uricase—Biozyme; Cat No. U5
(4-(2-hydroxyethyl)-1-piperazineethanesulfonic acid) (HEPES sodium salt)—Fisher Scientific—Cat No. BP410-1
Sodium Chloride (NaCl)—Fisher Scientific; Cat No. S7653
Poly(methylene-co-guanidine HCL) (PMG)-5K MW, Scientific Products Polymer Inc; CAS No. 55295-98-2
Polyacrylic Acid—Sigma Aldrich; CAS-90003-01-4
Trizma Base—Sigma Aldrich; Cat No. T4661
Hydrochloric acid (HCl)—Sigma Aldrich; CAS No. 7647-01-0
Sodium Hydroxide (NaOH)—Sigma Aldrich; CAS No. 1310-73-2
L-Histidine (His)—Sigma; Cat No. H8125
L-Aspartic Acid (Asp)—Fluka; Cat No. 30090
L-Cysteine (Cys)—Fluka; Cat No. 11189
Chymotrypsin—Sigma; Cat No. C3142
Uric Acid—Fisher Scientific; Cat No. AC17129-0250
Polyethylene glycol (PEG) 300—Fluka; Cat No. 81160

### Equipment

Vortex—Fisher Scientific, Vortex Mixer
Centrifuge—Eppendorf, Centrifuge 5804
Conical tubes—Fisher brand, 50 mL
UV-Vis Spectrophotometer—Agilent 8453
Software—UV-Visible ChemStation, Agilent, B.01.01[21]
Filter—Nalgene 500 mL 455-0500
pH meter—Fisher Scientific, Accumet Excel—XL60
Stir Plate—Corning Stirrer, PC-220
UV Cuvettes—PlastiBrand; Cat No—7591-70
Microcentrifuge tubes—Fisher Scientific; Cat No. 05-406-16
Heating block—Eppendorf Thermomixer R, Cat No. 022670000

### Preparing Buffers and Reagents

Buffers and polymers were prepared according to the following and stored at room temperature.

### Preparing Formulation Buffers

A concentrated stock of 1 M HEPES/1 M NaCl pH 8.0 was prepared by dissolving 119.2 g HEPES and 29.2 g in 500 mL DI water, then adjusting the pH accordingly.

1 L of 25 mM HEPES/25 mM NaCl pH 7.2 was prepared by mixing 25 mL of the aforementioned 1 M stock with 975 mL of DI water, adjusting the pH to 7.2 with 6 N HCL, then filter-sterilizing with a 0.2 μm sterile filter bottle. 1 L of formulation buffer (12.5 mM HEPES/12.5 mM NaCl/50% PEG 300) was then prepared by mixing 500 mL of the 25 mM HEPES/25 mM NaCl stock with 500 mL of 100% PEG 300.

### Preparing Polymer Stocks

500 mL of 5% PMG was prepared by mixing 83.3 mL of 30% PMG with 416.7 mL of 25 mM HEPES/25 mM NaCl pH 7.2. The pH of this solution is around 3, and needed to be adjusted to 6.2 through addition of 6 N NaOH while stirring on a stir plate.

500 mL of 5% PAA was prepared by mixing 25 mg of dry PAA into 500 mL of 25 mM HEPES/25 mM NaCl pH 7.2.

### Preparing Assay Buffer

The buffer used for the uricase assay was 50 mM boric acid, 0.13 mM uric acid, pH 8.0. A 50 mM stock of boric acid buffer was prepared by dissolving 3.09 g boric acid in 1 L DI water, adjusting the pH to 8.0, and filtering through a 0.2 μm filter. 4.37 mg of uric acid was dissolved into a 20 mL aliquot of the boric acid stock solution to make a 1.3 mM uric acid solution. 10 mL of this solution was added to 90 mL of the 50 mM boric acid stock to make the final assay solution of 0.13 mM uric acid. 3 mL of 50 mM boric acid buffer was aliquoted into a UV cuvette and the spectrum was blanked at 292 nm 3 mL of 0.13 mM uric acid assay buffer was added to another cuvette and the absorbance at 292 nm was read. An A₂₉₂ₙₘ of 1.4-1.6 is acceptable. If the reading is lower or higher than this range, adjust the 0.13 mM uric acid assay buffer by adding more 1.3 mM uric acid or diluting with 50 mM boric acid buffer respectively. Fresh 1.3 mM and 0.13 mM uric acid solutions were made daily.

### Preparing Amino Acid Buffer for pH Stability Assays

Into 100 mL of DI water, 388 mg of His, 332.8 mg Asp, and 302.9 mg Cys were dissolved, making the final concentration of each amino acid 25 mM. 10 mL was aliquoted into each of 7 tubes, which had been designated for a specific pH—1.5, 2, 3, 4, 5, 7, and 8. The pH was adjusted for each buffer tube, respective to its designation, by adding 1 N HCl or 1 N NaOH.

### Application of First Coat: PMG

Crystal coating was done in 45 mL batches in 50 mL conical tubes. First, 13.3 mL (450 mg) of biozyme crystals in crystallization buffer (33.9 mg/mL) were aliquoted into a 50 mL conical tube. Next, 27 mL (1.35 g) of the 5% PMG stock was mixed in a separate 50 mL conical tube with 4.7 mL of formulation buffer by vortexing on max speed for 10 sec. PMG+buffer solution was mixed into the crystal solution by vortexing together at medium speed for 10 sec. The resulting crystal concentration was 10 mg/mL, in a total volume of 45 mL. The final weight ratio was 1:3 (uricase: PMG). The tube was put on end-over-end rotator for ˜5 min, and then spun down for 5 min at 2500 rpm in the table-top centrifuge. The supernatant was decanted from the pellet and discarded.

### Application of Second Coat: PAA

In a 50 mL conical tube, 3.9 mL (1.89 g) of the 5% PAA stock was mixed with 41.1 mL formulation buffer. This solution was added to the crystal pellet, which was carefully resuspended with gentle vortexing and pipetting up and down with a 10 mL pipette. The tube was put on the end-over-end rotator for ˜5 min, then spun down at 2500 rpm for 5 min. The supernatant was decanted from the pellet and discarded.

To the pellet, a minimal volume of formulation buffer was added to resuspend via vortexing. The final concentration of coated uricase crystals, once all the batches were pooled, was 47.3 mg/mL in 165 mL total.

### Confirmation of Coating and Crystal Stability

Three tubes were prepared; one with 195.8 μL pH 7 amino acid buffer, one with 195.7 uL of pH 10.5 buffer (acquired from N. Khalaf), and one with 195.8 μL of pH 3 amino acid buffer. To each of these tubes, 4.2 μL of coated crystals from the final stock were added and vortexed, each tube containing 1 mg/mL uricase. A drop from each tube was put on a slide and analyzed under the light microscope.

### Uric Acid Assay General Procedure

A kinetics assay was set up to run at 37° C., with data collection at an absorbance of 292 nm and a light path length of 1 cm. Data points were collected at intervals of 2.5 sec for a total run time of 200 sec. One UV compatible cuvette was filled with 3 mL of 50 mM boric acid buffer and blanked 3 mL assay buffer (50 mM boric acid+0.13 mM uric acid) was added to cuvettes with mini stir bars. 10 μL of sample was added to the 3 mL assay buffer and assay was run with continuous stirring. ΔA₂₉₂ₙₘ was calculated using the most linear part of the graph of AU/sec, which was usually the entire run (from 0-200 sec). Units of activity (defined as micromoles of substrate degraded per minute per mg of enzyme) were calculated according to the following equation:

$$Units = \frac{( Δ {A}_{292 nm} \text{/} min Test Sample ) * 3}{12.2 * ( mg uricase )}$$

The equation is multiplied by 3 since the reaction is in a 3 mL volume. It is divided by 12.2 because this is the millimolar extinction coefficient for uric acid at 292 nm. Usually 0.001 mg of uricase was used per assay, which corresponds to approximately 0.015 U. Anywhere from 0.001 U to approximately 0.03 U can be used in this assay.

### pH Stability Assays

Crystallized Biozyme uricase formulated with PMG and PAA as described above was assayed for both pH and chymotrypsin stability. For pH stability, five 1 mg/mL solutions of coated uricase crystals were prepared in amino acid buffer, one of each for pH 1.5, 2, 3, 4, and 5. One solution was prepared in formulation buffer as a control. To prepare the 1 mg/mL solutions, 500 μL of the formulated PAL was first adjusted to a concentration of 30 mg/mL by adding 288.3 μL of formulation buffer. 6.67 μL of this 30 mg/mL formulated crystal stock was added to 193.33 μL of amino acid buffer, pH 1.5-5, in five different microfuge tubes. 6.67 μL of the crystal stock was also added to 193.33 μL of formulation buffer for the control. Preparation of these samples was staggered 10 min apart to ensure no overlap in time points. From each sample, 10 μL was added to 90 μL of 50 mM boric acid buffer to make 0.1 mg/mL solutions, and from these 10 μL was assayed immediately by adding directly to 3 mL of assay buffer and running the uricase kinetics assay described above. These data constitute the 0 h (wash) time points.

### Chymotrypsin Stability Assays

To prepare the chymotrypsin stock, 2 mg of chymotrypsin powder was weighed into a microfuge tube and 1 mL of formulation buffer was added to dissolve the protein. A 1 mg/mL solution of formulated uricase crystals was prepared from the 30 mg/mL stock by adding 16.7 μL of stock to 483.3 μL of 50 mM boric acid buffer. 5 μL of chymotrypsin stock was added to the 1 mg/mL uricase solution to achieve a final weight ratio of 1:50 (chymotrypsin:uricase). 10 μL of this solution was diluted into 90 μL of boric acid buffer, and 10 μL of this 0.1 mg/mL uricase solution was assayed immediately for the 0 h time point. The 1 mg/mL uricase+chymotrypsin solution was put on the heating block at 37° C. with 400 rpm shaking. 10 μL was removed, diluted, and assayed at each subsequent time point.

### Results and Discussion

Light microscope images show the effect of non-mother liquor buffers of various pHs on coated uricase crystals (data not shown). The coated crystals in pH 7 amino acid buffer resemble the uncoated crystals in crystallization buffer. Unlike the coated crystals, the uncoated crystals dissolve when removed from the crystallization buffer. The coated crystals were dissolved in extreme alkaline pH to show the polymer shells formed by the polyelectrolyte coating. The polymer layers retain the forms of the crystals, even after they have dissolved. Remarkably, in pH 3 the coated crystals are still mostly intact.

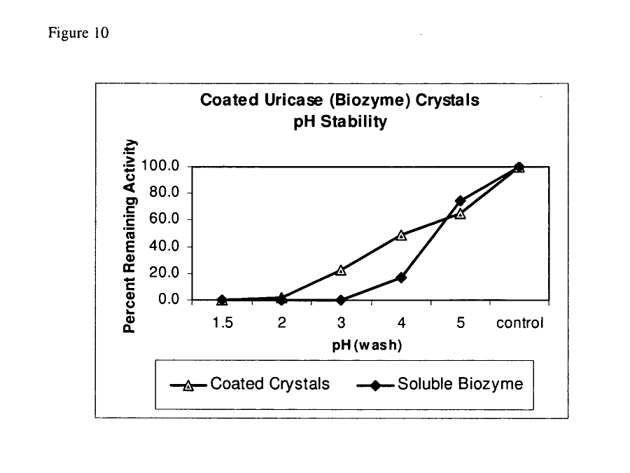

The following table contains the calculated percent specific activity retained for crystallized/coated and native Biozyme uricase after incubation at various acidic pHs. The percentages were calculated by setting 100% as equal to the control activity for each group. See also FIG. 10.

TABLE 23
pH stability of crystallized/coated Uricase

| pH | Activity (U/mg) | % Activity Retained |
|---|---|---|
| Coated Crystals | | |
| 1.5 | 0 | 0 |
| 2 | 0.302 | 2 |
| 3 | 3.111 | 23 |
| 4 | 6.731 | 49 |
| 5 | 8.894 | 65 |
| control | 13.648 | 100 |
| Soluble Biozyme | | |
| 1.5 | 0 | 0 |
| 2 | 0 | 0 |
| 3 | 0.04 | 0.3 |
| 4 | 2.23 | 17 |
| 5 | 10.01 | 75 |
| control | 13.38 | 100 |

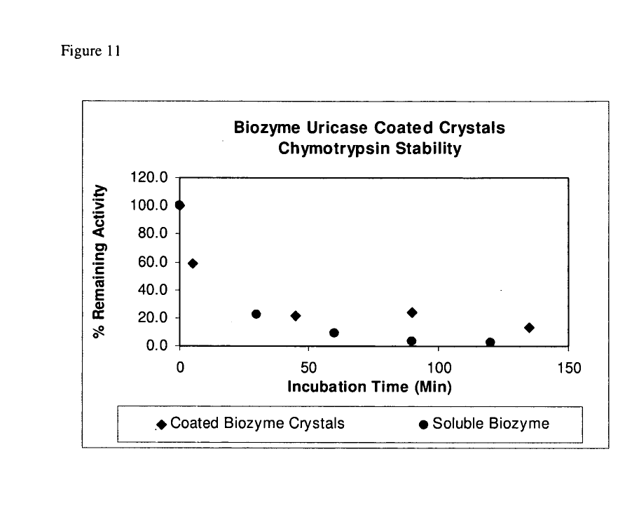

The following table contains the calculated percent specific activity retained for crystallized/coated and soluble Biozyme uricase after incubation with chymotrypsin. The percentages were calculated by dividing each activity value by the control value for each group. See also FIG. 11.

TABLE 24
Uricase activity after incubation with chymotrypsin

| Timepoint (Min) | Activity (U/mg) | % Activity Retained |
|---|---|---|
| | Coated Biozyme Crystals | |
| 0 | 13.648 | 100 |
| 5 | 8.041 | 59 |
| 45 | 2.894 | 21 |
| 90 | 3.270 | 24 |
| 135 | 1.770 | 13 |
| | Soluble Biozyme | |
| 0 | 11.262 | 100 |
| 30 | 2.554 | 23 |
| 60 | 0.991 | 9 |
| 90 | 0.417 | 4 |
| 120 | 0.242 | 2 |

### Conclusion

Coating uricase crystals with two polyelectrolyte layers (PMG and PAA) provides enough support to maintain crystalline structure when the crystals are exposed to non-mother liquor buffer.

The acid stability of the coated crystals is enhanced when compared to non-formulated, soluble biozyme uricase, at least for pH's greater than 2. Incorporation of PSS or dextran sulfate as the negative polymer coat mauy further increase chymotrypsin resistance, as these are chymotrypsin inhibitors.

### Example 19

### Pre-Clinical Studies

### Summary

This report is a summary of several non-clinical, non-GLP studies performed to demonstrateg efficacy of formulated uricase in urate oxidase knockout mice (UoxKO), a model for hyperuricemia and urate nephropathies.

The prevalence of hyperuricemia, or elevated plasma uric acid, has been increasing in Western countries over the last decade and correlates well with an increase in the prevalence of renal disease, gout, hypertension and metabolic syndrome. It occurs either as a result of excessive urate production or decrease in renal excretion of uric acid or both. Humans lack urate oxidase, an enzyme which degrades uric acid. Causes of hyperuricemia can be primary such as increased plasma uric acid levels due to high purine metabolism, especially in people that consume purine-rich diets, and secondary, when uric acid levels are significantly increased due to cell lyses such as in patients that have tumor lyses syndrome. Whether complications due to hyperuricemia develop depends on both the levels of uric acid and the duration. Hyperuricemia, is in fact considered as one of the biochemical hallmarks for gout, also called metabolic arthritis, that is a result of the deposition of monosodium urate crystals on the articular cartilage of joints, tendons, and surrounding tissues that provokes inflammation and severe pain. Gout is characterized by excruciating, sudden, unexpected, burning pain, as well as swelling, redness, and stiffness in the affected joint. Most people experience several gouty attacks per year with each subsequent attack being more painful and the interval between attacks being shorter.

Elevated uric acid levels are also associated with renal disease, kidney stones, hypertension, and disease linked to metabolic syndrome and obesity. There is a mounting evidence that hyperuricemia itself may be an independent risk factor for cardiovascular disease.

Presently, existing treatments for hyperuricemia and acute gout involve:

1) Uricostatic agents which inhibit xanthine oxidase, and lead to decrease production of uric acid. The most commonly used drug is allopurinol and its active metabolite oxypurinol (not approved in US). Recently febuxstostat, which is a non specific xanthine oxidase inhibitor, was approved in Europe (ADENURIC®), but pending approval in US.

2) Uricosuric agents, such as probenicid and sulfinpyrasone, which act on the renal uric acid anion transport pathway to increase uric acid excretion in the urine and therefore reduce plasma urate concentration.

3) Urate oxidases (uricase), such as PURICASE® (pegloticase) that presently is approved only for patients that are suffering from tumor lysis syndrome (TLS), but is in the investigation phase in patients with severe hyperuricemia and gout.

To some extent hyperuricemia and gout can be managed by changes to a patient's diet and lifestyle. A strict purine-free diet will reduce serum uric acid levels by 15 to 20% making it inefficient for patients whose levels are above 9 mg/mL. These patients have the highest incidence rate of gout.

All above mentioned urate lowering therapies have limited effectiveness or are not always well tolerated, provoking side effects such as severe hypersensitivity reactions (allopurinol) and kidney stones (probenicid). Therefore, as a substitute for or as a complement to existing therapies, we posited a new approach for treatment of hyperuricemia and gout and tested it as an orally delivered urate-specific enzyme (ALTU-242), that is stable and active in the pH and protease challenged environment of the intestine. We tested its efficacy on the reduction of plasma and urinary urate in uricase deficient mice (Uox^{-/-}), a model with severe hyperuricemia and urate nephropathy. We hypothesized that a stable and active urate-specific enzyme will reduce the body pool of urate by first degrading intestinal urate and promoting a blood to lumen transepithelial gradient that will enhance enteric excretion and thereby reduce plasma urate levels.

In this report we present the results from proof of concept efficacy studies performed with modified uricase (purchased from Amano Japan, Biozyme Laboratories, UK or in-housed fermented and formulated) on reduction of plasma and urinary uric acid in urate oxidase deficient mice (Uox−/−). We also compared the efficacy of formulated uricase or ALTU-242 with allopurinol, which is a specific xanthine oxidase inhibitor, and is the most commonly used therapy for hyperuricemic patients and also these patients with hyperuricosuria and uric acid stones.

Several increasing doses of uricase, either mixed with food or given by intraperitoneal injection (IP) were tested in mice with severe hyperuricemia. To slow the disease progression, all mice before the birth and after the birth were maintained on allopurinol (50-150 mg/mL) that was given with the drinking water.

Initially, as a proof of pharmacological principle, Uox−/− mice were IP injected once, with 30 U or soluble, crystalline uricase or placebo and efficacy was monitored on reduction of plasma uric acid up to 24 h post-injection time.

Next, as a proof of physiologic principle, oral therapy with 5, 25, 100, 200 mg/mouse/day of formulated uricase (specific activity from 10-20 U/mg, depending on the supplier and formulation) was tested in the hyperuricemic Uox−/− mice for up to 4 weeks. Further more, we compared the standard dose of allopurinol (0.7-1 mg/day) with ALTU-242 (200 mg/day). Efficacy of the daily oral treatment was monitored by reduction of plasma and urinary uric acid compared to placebo treated controls.

Results from these non-clinical, non-GLP studies clearly demonstrated that oral treatment with formulated uricase (ALTU-242) at the dose ˜200 mg (2000 U/d/mouse) produced significant and sustained reduction in hyperuricemia and hyperuricosuria, while lower daily doses of 50 mg and 100 mg (500 and 1000 U/d/mouse) were only able to reduce urinary urate significantly, with no visible changes in plasma uric acid levels.

### Introduction

Hyperuricemia is the presence of high levels of uric acid in the blood. It is the result of urate overproduction (10%), under excretion (90%), or often a combination of the two. Humans lack urate oxidase, an enzyme which degrades uric acid. Causes of hyperuricemia can be primary (increased uric acid levels due to purine metabolism), and secondary (high uric acid levels due to another disease or condition). Consumption of a purine-rich diet (high protein and fat and beer) is one of the main causes of hyperuricemia in Western World. More then 10% of adults in the U.S population are documented to have hyperuricemia at least once in their lifetime, however most do not need further workup or treatment. Whether complications develop will depend on both, the level and the duration of hyperuricemia. Elevated plasma uric acid is a predisposing condition for gout, and is also intricately linked with hypertension, glucose intolerance, dyslipidemia, insulin resistance, truncal obesity, and cardiovascular disease.

Gout, or metabolic arthritis is a disease resulted from the deposition of monosodium urate crystals on the articular cartilage of joints, tendons, and surrounding tissues once concentrations of uric acid in the blood stream is above its solubility of 6.8 mg/dL. The needles of urate crystals cause an inflammatory reaction in the surrounding tissues. Typically, persons with gout are obese, predisposed to diabetes and hypertension, and are at a higher risk of heart disease. Alcohol intake often causes acute attacks of gout and hereditary factors may also contribute to the elevation of uric acid.

Gout is characterized by excruciating, sudden, unexpected, burning pain, as well as swelling, redness, warmth, and stiffness in the affected joint. Gout usually attacks the big toe (approximately 75 percent of first attacks however, it also can affect other joints such as the ankle, heel, instep, knee, wrist, elbow, fingers, and spine. 93% of people will experience a second gout attack after an initial attack, and 60% will experience it within one year. After repeated attacks of gout each subsequent attack is often more painful and the interval between attacks is shorter.

Hyperuricemia is a common feature in gout, although urate levels are not always raised. However, a high uric acid level does not necessarily mean a person will develop gout.

There are also different propensities to develop gout. In the United States, gout is twice as prevalent in African American males as it is in European-Americans. A seasonal link also may exist, with significantly a higher incidence of acute gout attacks occurring in the spring. Gout affects mostly men between the ages of 50 and 60. Gout is more common in affluent societies due to a diet rich in proteins, fat, and alcohol.

There are four stages of gout:

(1) Asymptomatic hyperuricemia (no symptoms, but gout starts to form)

(2) Acute gout (gouty arthritis, pain and swelling occurs due to deposit of monosodium urate crystals)

(3) Interval/intercritical gout (gout attacks are subsided)

(4) Chronic tophaceous gout (permanent damage of the joints and in some cases of kidneys)

It is estimated that 18% of the population in US suffer from hyperuricemia, and around 1% of all develops gout. The current gout population in the U.S. is estimated at 3 to 5 million. The incidence rate for gout varies with the urate level with the highest incidence occurring at the highest hyperuricemia.

Hyperuricemia can also be linked with increased excretion of uric acid that can cause the development of uric acid kidney stones. The incidence rate of uric acid stones is in the U.S. is approximately 0.5% per year or 5-10% of all other kidney stones.

Lowering of the concentration of uric acid in plasma and urine is an important part of medical treatment, because this reduction changes the supersaturation index and lowers/prevents formation of monosodium urate crystals. We are developing oral therapy for hyperuricemia and gout with specific modified uricase from Candida sp. that exerts its effect along the gastrointestinal tract. This enzyme has high specificity to degrade uric acid to soluble allantoin and hydrogen peroxide (H₂O₂). The therapeutic effect of the enzyme is measured as a reduction in plasma and urinary uric acid. This reduction can be seen only if the enzyme survives the low pH of the stomach and exposure to proteases along the GI tract. It is postulated that formulated uricase once in the gut, will first break down uric acid excreted into the small intestine from circulation (˜30% from daily production) and with the time will enhance enteric elimination due to uric acid concentration gradient between circulation and the intestine. Ultimately, this advance elimination of uric acid via the intestine will result in the reduction of plasma uric acid.

In this example, results from several experiments are presented. Different doses of soluble or formulated uricase were tested in the Uox−/− mice that were pre-treated with allopurinol. During the each study multiple, blood and 24 h urine samples were collected for the assessment of uric acid levels. Additional parameters monitored were mortality checks, food and water intake together with body weights. At the end of the experiment mice were sacrificed and histopathology was performed on the kidneys.

### Objective

The objective of this non-GLP study was to demonstrate the positive effect of formulated uricase on the reduction of plasma and urinary urate in a mouse model with severe hyperuricemia and urate nephropathy that partially mimics human disease.

### Study Information

### Regulatory Compliance

This study was conducted in compliance with procedures involving the care and use of animals that was reviewed and approved by the Institutional Animal Care and Use Committee (IACUC) prior to conduct. During the study, the care and use of animals was in accordance with the principles outlined in the current Guide to the Care and Use of Experimental Animals as published by the NIH. This was a non-GLP study.

### Materials and Methods

### Test Material

### Identification and Composition

The test article used in this program was soluble or formulated crystalline uricase from Candida sp. (Uricase, urate: oxygen urate oxidoreductase E.C. 1.7.3.3). In our experiments we used uricase purchased from Amano, Japan (Cat No=UR-2, lot #URF0451013, URF1050146, URG0150804, URF 0753109), uricase purchased from Biozyme, UK (Cat No# U5, Batch 8, 14,16) and in house expressed, purified and formulated (see Example 18). Measured specific activity for different batches were 10±2 U/mg A₂₈₀, 13-16 U/mg A₂₈₀ of and 20-25 U/mg A₂₈₀ for uricase from Amano, Biozyme and in house enzyme, respectively.

### Storage Conditions

Different formulations of uricase were stored in tightly sealed containers at 2-8° C.

### Assay of Test Materials

To measure specific activity of uricase we used a modified enzymatic assay described previously by http://pointescientific.com/. One unit of enzyme activity was defined as a conversion of 1 μmol substrate to product per 1 min at 37° C. at pH 8.

### Animals and Animal Care

### Animals

In all studies described in this report we used knock out mice that are lacking urate oxidase gene (Uox−/−) described previously by Wu X. at al in 1994. Cryopreserved embryos (JAX stock, strain name B6; 129S7-Uox^{tm1Bay}/J, stock number 002223) were recovered at JAX laboratory.

Uox−/− mice develop severe hyperuricmia, with the uric levels being up to 10 fold above normal resulting in deposition of uric acid and severe nephropathies. Mice do not develop gout because of the rapid accumulation of monosodium urate crystals in the kidneys that provoke severe nephropathies and kidney failure. 65% of null mice died soon after birth, but sudden death of young mice can be prevented with administration of allopurinol (50-150 mg/L supplemented in the drinking water).

### Housing

Animals were housed individually in metabolic cages (Tecniplast metabolic cages, #3700M071, 21020 Buguggiate (Va), Italy), or in groups of 4 per cage.

Each mouse was clearly identified with a color-coded cage card indicating study number, animal number, sex, species, group number and dose level. No other animal species were housed in the same room. The animal room and the cage cleaning were performed throughout the study according to the protocol set up in the performing laboratory's procedure. Care and use of the mice was according to the conditions specified in the Animal Welfare Act and as described in the Guide for the Care and Use of Laboratory Animals.

### Acclimation

All mice were acclimated approximately a week before the start of the experiment.

### Monitoring and Assessment

Body weights were obtained before the experiments and at the end of the experiment. The animals were also assessed for signs of poor health and other conditions that might interfere with the experimental results.

### Diet and Water

Mice were fed the mouse lab diet (A04, France or 5LL4 Lab Diet, supplied to by Dean's feed for Purina, or so called Jackson Laboratory mix). Drinking water for all mice before the birth and after the birth was supplemented with allopurinol (50-150 mg/L) ad libitum. After the pre-treatment period, age matched male and female mice were divided into groups based on plasma and urine uric acid levels. Soluble or formulated uricase was given to mice either as an IP (intraperitoneal injection), 30 U/mouse (soluble uricase (20 U/mg)) or orally as food/enzyme mix (nominal dose 5, 50, 100, 200 mg/3.5 g food ad libitum). The placebo-treated group was IP injected with formulation buffer or was fed with the food without the test article. During the studies, mice were given a fresh food/enzyme mix ad libitum every day. Concentrations of contaminants in the diet (e.g., heavy metals, aflotoxin, organophosphates, chlorinated hydrocarbons and PCBs) were controlled and routinely measured by the manufacturer (Harlan, Teklad, Madison, Wis.). It was assumed that there were no known contaminants in the diet and water that would interfere with the assessment of the efficacy of the test articles.

### IP Injection of Soluble Uricase Experimental Design

As a proof of pharmacological principal we administrated soluble uricase (Amano lot# URF0451013) to Uox−/− mice that were kept without allopurinol for 5-7 days prior to treatment. Always, before the study, mice were randomized into treatment and control groups based on their plasma and urine urate levels. Soluble uricase was given intraperitonealy (IP) to mice at the dose of 30 U/mouse (specific activity of soluble uricase is 20 U/mg). Each animal served as its own control.

Oral Treatment with Soluble or Crystalline Uricase/Experimental Design

As a proof of physiological principal, hyperuricemic male and female Uox−/− mice were treated orally with soluble uricase from Amano, specific activity ˜10 U/mg of lyo powder, lot# URF1050146) or crystalline coated uricase specific activity 13 U/mg, see Example 18). For this assessment, age matched male and female mice were pre-treated with allopurinol (50-150 mg/L) to control the level of hyperuricemia and prevent deposition of monosodium crystals in the kidneys. Always, before the study, mice were randomized into treatment and control groups based on their plasma and urine urate levels. Efficacy of the treatment was monitored by changes in plasma and urine uric acid. Mice were fed with different doses of uricase (5, 50, 100 and 200 mg/day) or placebo, that was mixed with the food and administered to Uox−/− mice for up to three weeks. If not specified, during all studies the diet and/or water were supplemented with 1% bicarbonate to reduce the acidity of the stomach. Each morning the mice feeders were re-filled with ca. 7 g of the food/enzyme mix.

### Animal Sorting

The animals were sorted into groups by body weights and plasma and urine uric acid levels. Each group contained n=5-11 mice.

### Blood and Urine Collection

Once a week blood was collected by RO (50 μL) and processed to LiHep plasma.

Diuresis (16-24 h urine samples) was measured in all mice placed in metabolic cages once, twice or three times per a week.

### Plasma and Urine Uric Acid Assessment

Plasma and urine samples were collected at 9-10 am on the day of collection. The volume was measured, and typically urine and plasma samples were stored frozen at −20° C. until assayed for uric acid. BioAssay System (QuantiChrom Uric Acid Kit (DIUA-250, BioAssay System, 3423 Investment Boulevard, Suite 11, Hayward, Calif. 94545, US) was used for the uric acid estimation. Plasma uric acid was calculated based on the used standards uric acid standards.

The uric acid levels in the urine were expressed as the amount of urate excreted in the urine during a 16-24 h collection period:

(mg/dL uric acid×urine volume (mL))/100=mg/24 h

### Statistical Analysis

Statistical analysis was performed on the data generated from these experiments by using unpaired two-tailed Student's t-test. Differences were considered significant if p≦0.05. In the text, mean standard error (±se) are listed.

### Renal Histology

Mouse kidneys were routinely processed for paraffin embedding and positioned in order to obtain a complete cross section of the kidney. Each kidney was cut in 12 serial sections at 4 μm per kidney and stained with either hemotoxylin-eosin for routine histological examination. Urate crystals are water soluble, thus when processed with hemotoxylin-eosin histology, most of them disappear during the formaldehyde step. What is then seen are the tubular atrophies, inflammation and fibrosis, and only occasionally it is possible to see a crystal.

Therefore in many cases, part of the kidney was placed in ethanol, avoiding aqueous solutions. Then those sections were stained only with alcoholic-eosin, instead of no hematoxylin.

### Results and Discussion

Interperitoneal Injection (IP) of Soluble Uricase in Uox−/− mice

In this study, we administrated soluble uricase to Uox−/− mice. To increase the severity of hyperuricemia, allopurinol (50 mg/L in drinking water) was removed 5 days prior to the study. Before the dosing mice were randomize per body weight and plasma uric acid levels into control and uricase treatment groups. Soluble uricase was given by IP to mice at a dose of 30 U/mouse. Each animal served also as its own control. The table below shows the experimental design summary:

TABLE 25
Effect of Single Dose of Soluble Uricase or Crystalline Uricase in Uox−/−Mice

| GROUPS | | | URINE COLLECTION |
|---|---|---|---|
| UOXKO MICE | TREATMENT | | 18 H |
| (-ALLOPURINOL, 5 D) | SOLUBLE/CRYSTALLINE | BLOOD | (STARTING 4 H POST |
| N = 4-6 (MIXED SEX) | URICASE | COLLECTION | INJECTION TIME) |
| CONT | saline | 0, 0.5, 4, 24 h | + |
| Sol Uricase | + | 0, 0.5, 4, 24 h | + |
| ~30 U (Spe. Act. 16 U/mg) | | | |

Within 30 min, plasma uric acid levels were completely normalized in the mice injected with 30 U of soluble uricase (2.57±0.17 mg/dL vs. 7.24±1.15 mg/dL) and stayed within the normal range up to 4 h (2.57±0.17 mg/dL) post injection time.

Therefore, we clearly demonstrated that uricase dose of 30 U efficiently degraded elevated plasma uric acid levels.

We also demonstrated that the given dose of 30 U was able to significantly reduce excretion of urate. 4 h after dosing mice were placed in metabolic cages and urine was collected for 18 h.

Uricase treatment significantly reduced hyperuricosuria in the treatment groups when compared to placebo treated control. Excretion of urate normalized 24 h after IP injection of soluble uricase (1.28±0.26 mg/18 h soluble vs. 4.47±0.44 mg/18 h control).

Oral Treatment with Formulated Uricase in Uox−/− Mice

As a proof of physiological principal, hyperuricemic male and female Uox−/− mice were treated orally with soluble uricase (Amano) with three different doses of 5, 50, and 200 mg/day, n=7 or placebo. Uricase was mixed with the food and administered to Uox−/− mice for up to three weeks. Diet and water was also supplemented with 1% bicarbonate to reduce acidity in the gastric compartment of mice. Additionally, all mice were kept on allopurinol (50 mg/L) during the breeding and post-weaning periods. To increase the hyperuricemia, allopurinol was removed from the drinking water one week before the study. Initially we tested 200 mg/day and compared with the placebo treated controls. Subsequently, we performed dose ranging study and tested efficacy of nominal doses of 5 and 50 mg/day of uricase.

TABLE 26
Effect of Different Doses of Uricase in Uox−/−Mice

| | | TREATMENT |
|---|---|---|
| GROUPS | | SOLUBLE URICASE A (10 ± 2 U/MG LYO POWDER)* |
| UOXKO MICE | | (5, 50, 200 MG/3.5 G FOOD) & 1 MG CATALASE* |
| (-ALLOPURINOL, 7 D) | | (SIGMA 4000 U/MG) & 1% BICARBONATE IN WATER AND |
| N = 8 MIXED SEX | | DIET FOR ALL GROUPS |
| DURATION | | ~3 WEEKS |
| CONT | | diet |
| 200 | mg Uricase | Diet/enzyme mix |
| 50 | mg Uricase | Diet/enzyme mix |
| 5 | mg Uricase | Diet/enzyme mix |

*allopurinol was removed from drinking water 7 days prior dosing
**catalase was supplemented to quench H₂O₂

Efficacy of the different doses of uricase (5, 50 and 200 mg/d/mouse that equals to approximately 50, 500 and 2000 U/d/mouse) on reduction of hyperuricemia and hyperuricosuria were assessed by monitoring changes in plasma and urine uric and compared with placebo treated controls. Blood and urine samples were collected once per week during the study period.

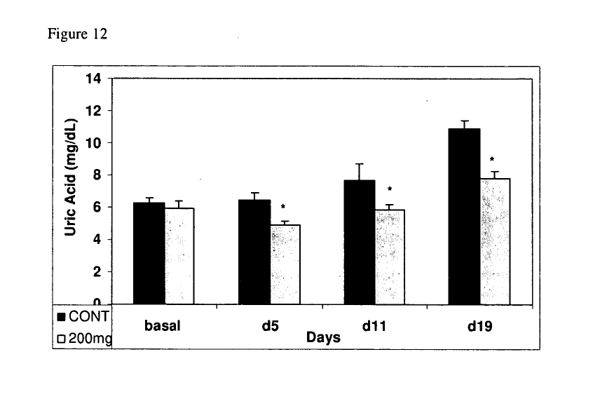

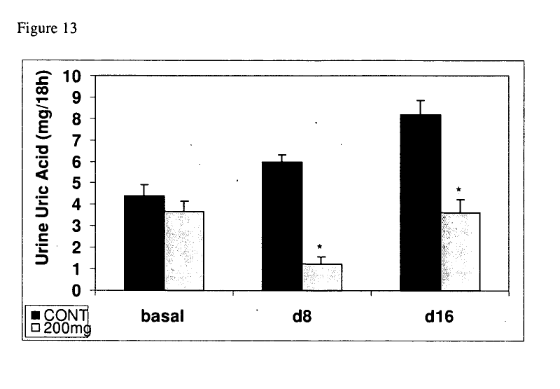

As shown in FIG. 12 and FIG. 13, uricemia and uricosuria were reduced considerably, upon daily oral administration of 200 mg (˜2000 U) of uricase, when compared to untreated controls during the 19 days treatment.

Changes in plasma uric acid reflected also uric acid excretion. Mice fed daily with 200 mg (2000 u/d) of uricase for 19 days had a mean overall reduction in urinary urate of 66% (2.45±0.77 mg/18 h vs. 7.1±0.49 mg/18 h, p<0.05, FIG. 13) and plasma urate was reduced 26% (6.19±0.68 mg/dL vs. 8.35±0.68 mg/dL, p<0.05, FIG. 12).

Mice fed 200 mg (2000 u/d) of uricase for 19 days had a mean overall reduction in urinary urate of 66% (2.45±0.77 mg/18 h vs. 7.1±0.49 mg/18 h, p<0.05, FIG. 13) and plasma urate was reduced 26% (6.19±0.68 mg/dL vs. 8.35±0.68 mg/dL, p<0.05, FIG. 12).

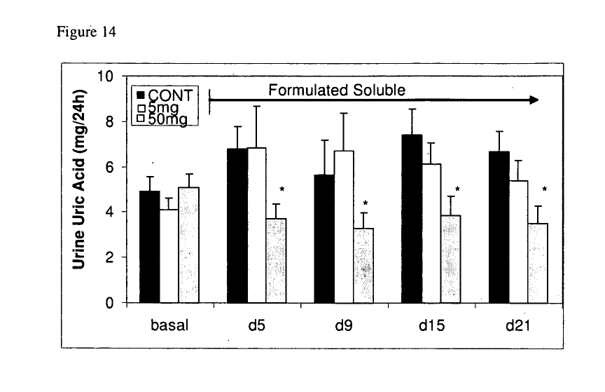

Using the same model, in a separate experiment two doses of uricase of 5 mg/day and 50 mg/day where compared with the untreated controls for 22 days. With the 50 mg (500 u/d) dose we demonstrated a constant reduction in uricosuria of 46% (3.59±0.74 mg/24 h vs. 6.63±1.15 mg/24 h, p<0.05, FIG. 14), while the lower dose of 5 mg (100 U/d) had minimal or no effect, implying specificity of the drug action (FIG. 14).

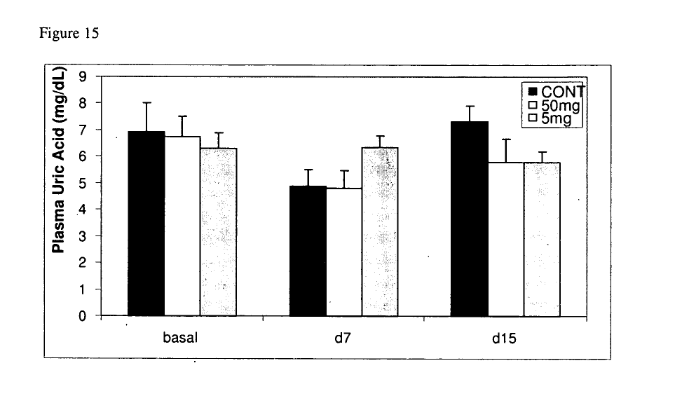

Interestingly these two doses did not reduce plasma uric acid, when compared to placebo treated control (FIG. 15), indicating a dose dependant effect of uricase action in the intestine on the reduction of hyperuricemia and excretion of uric acid in the urine.

At the end of the studies all mice were scarified and kidneys were processed for histopath analysis. In the majority of mice either from control group and treatment group mild hydronephrosis and multifocal cortical tubular atrophy and collapse of nephrons were detected. This finding was expected since mice had pronounced hyperuricemia despite the allopurinoll treatment.

Soluble Uricase Needs to be Protected from the Low pH of the Stomach

To further test the stability and efficacy of uricase in vivo, 1% sodium bicarbonate was removed from the food and water of Uox−/− mice. The experimental design summary is shown in the table below. Initially, a series of in vitro experiments have shown that soluble uricase is unstable below pH 6.

TABLE 27
Effect of 100 mg of Uricase in Uox−/− Mice w/wo 1% Bicarbonate

| | TREATMENT |
|---|---|
| GROUPS | SOL URICASE (~10 U/MG)* |
| UOXKO MICE (n = 8-12) | DAILY URICASE/FOOD MIX |
| DURATION | 18 DAYS |
| 1. CONT | diet |
| 2. 100 mg Uricase + catalase (1 mg, 400 U/mg) | + |
| 3. 100 mg Uricase + catalase (1 mg, 400 U/mg) & | + |
| 1% bicarbonate food and water | |

*given formulation of uricase has by weight 50% excipients
**catalase was supplemented to quench H₂O₂

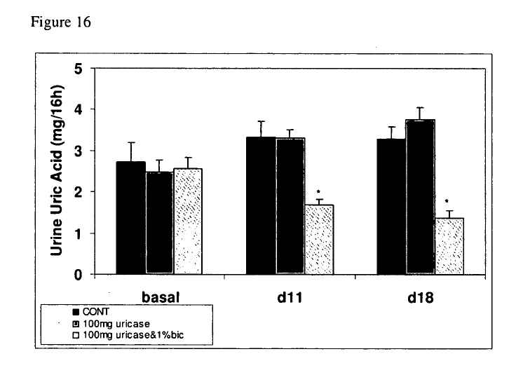

Efficacy of the 100 mg uricase (1000 U/day) on reduction of hyperuricemia and hyperuricosuria were measured by the changes in plasma and urine uric acid and compared with placebo treated controls during 18 days. Urine samples were collected once per week during the study period. 36 days prior to the study allopurinol was removed from drinking water of mice (FIG. 16).

As shown in FIG. 16 only mice fed with 100 mg (1000 U/day) uricase that was mixed with 1% bicarbonate show mean overall reduction in urinary urate of 45% (1.54±0.15 mg/16 h vs. 3.32±0.0.34 mg/16 h, p<0.05) during 18 days with a tendency towards normalization. Contrary, mice fed only with 100 mg uricase (1000 U/d) excreted the same high levels of uric acid as the placebo treated control.

This indicates that soluble uricase is sensitive to the low pH in the stomach, and that it needs to be further stabilized for effective degradation of uric acid in the intestine that comes from enteric excretion. This result is not surprising based on our in vitro studies previously performed.

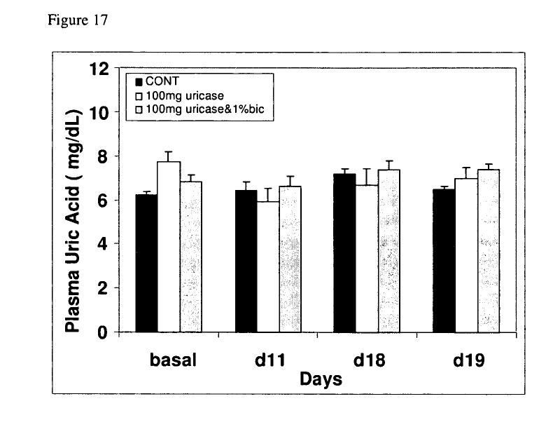

In addition, mice treated with uricase, despite having significant and sustained reduction in urine uric acid had the same high levels of urate in plasma as untreated controls (FIG. 17). This again indicates that oral uricase that exerts its effect solely in the intestine was able to degrade increased urate pool in plasma only to certain extent as was detected by reduction in urine urate. A higher dose of uricase is needed to positively affect plasma uric acid levels and further enhance enteric elimination as shown when the 200 mg dose was used.

Comparison Study: Allopurinol vs. Uricase

In the next experiment, a nominal dose of 200 mg/d of uricase (Amano, UrA) was compared to allopurinol (200 mg/L) for 10 days. The experimental design summary is shown in Table 28.

TABLE 28
Comparison of Allopurinol and Uricase in Hyperuricemic Uox−/− Mice

| | PRE-TREATMENT | |
|---|---|---|
| GROUPS | ALLOPURINOL | |
| UOXKO MICE | (200 MG/L WATER) | TREATMENT 10 DAYS |
| N = 8, EXP | ~1 MG/DAY WATER | ALL MICE FOOD IS SUPPLEMENTED WITH |
| CONTCONT (N = 6) | BEFORE/AFTER BIRTH | 1% BICARBONATE |
| CONT | + | |
| Allopurinol (20 mg/dL) | + | Allop~1 mg/day (20 mg/dL) |
| (14000 mg/day/70 kg) | | |
| 200 mgUricase + | + | Uricase (200 mg/3.5 g food) |
| Catalase | | Catalase (3 mg/3.5 g food Sp. act |
| | | ~13000 U/mg) |
| CONTCONT | + | just food no bicarbonate |

*given formulation of uricase has by weight 50% excipients, uricase from Amano
**catalase was supplemented to quench H₂O₂

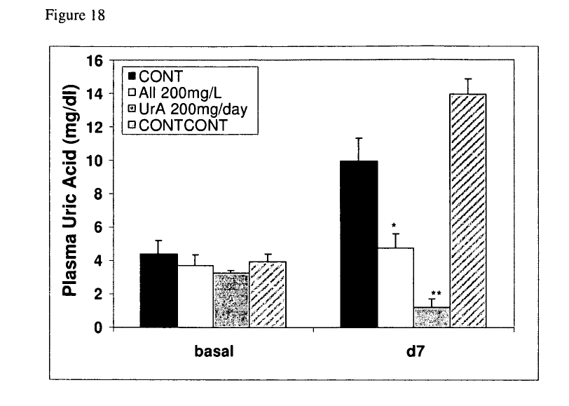

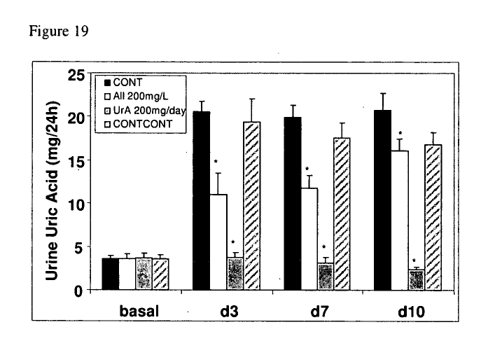

All mice treated with uricase (Amano, Lot# URG0150804) had reduced plasma and urinary uric acid, as shown in FIG. 18 and FIG. 19. The mean reduction in plasma urate and urine urate was 82% and 85% respectively (1.18±0.52 mg/dL vs. 9.93±1.37 mg/dL and 3.1±0.47 mg/24 h vs. 20.44±1.95 mg/24 h, p<0.05 for each) when compared to placebo control mice. In the allopurinol group, plasma and urinary urate reduction was less pronounced and was 53% and 47% respectively compared to placebo controls (4.74±0.87 vs. 9.93±1.37 mg/dL and 12.97±1.77 vs. 20.44±1.95 mg/24 h, p<0.05 vs. placebo). Mice that were fed regular diet without bicarbonate, CONTCONT group had plasma and urine urate in the same range as control mice fed food supplemented with 1% bicarbonate (FIG. 18 and FIG. 19).

Thus, this data clearly demonstrates that oral therapy with 200 mg uricase (2000 U) was able to normalize plasma uric acid in mice (normal levels in rodents are 1-2 mg/dL) while an extremely high-dose of allopurinol (1 mg/day) was not so effective.

In addition, during 10 days of treatment with uricase (2000 U/day), uric acid excretion normalized, while the effect of allopurinol was minimal as shown in FIG. 19.

Thus, this data clearly demonstrates that soluble uricase, protected from low gastric pH, had a significantly greater hypouricemic and hypouricosuric effect then allopurinol given at an extremely high dose of almost ˜1 mg/day.

### Efficacy of Crystalline Uricase in Uox−/− Mice

Uricase was further formulated by crystallization and coating. We used uricase from Biozyme (E. coli expressed uricase) that was crystallized with high salts (0.1 MgCl) and PEG8000. Coating of crystal was done with two polymers: poly methylene-co-gunidine HCL) and polyacrylic c acid in the ratio 1:3:4.2 (see Example 18). Efficacy was tested in Uox−/− mice that were kept on allopurinol (20 mg/dL in water). The experimental design summary is shown in the table below.

TABLE 29
Efficacy of Crystalline Coated Uricase (Biozyme)

| | PRE-TREATMENT | |
|---|---|---|
| | ALLOPURINOL | TREATMENT- 1 WEEK |
| GROUPS | (200 MG/L WATER) | COATED CRYSTALLINE URICASE BIOZYME |
| UOXKO MICE | ~1 MG/DAY WATER BEFORE/AFTER | SPEC. ACT~13 U/MG |
| N = 8 | BIRTH | 1% BICARBONATE WATER/FOOD |
| CONT | + | diet |
| ~100 mg Ur B | + | Uricase (~100 mg/3.5 g food) |
| | | Catalase (1 mg/3.5 g food, Sp. Act |
| | | ~13000 U/mg) |

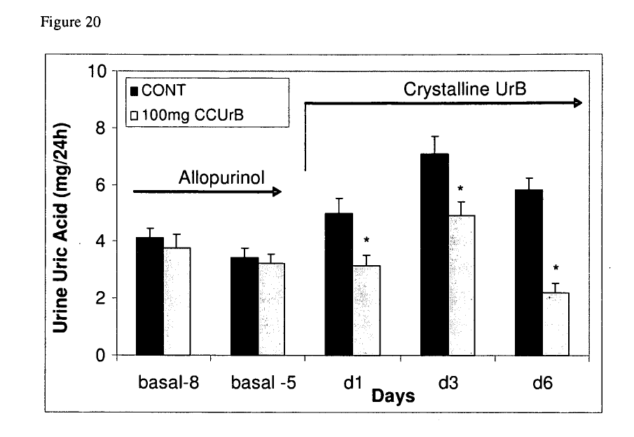

As shown in FIG. 20, daily oral administration of crystalline coated uricase (˜1000 mg/day) to Uox−/− mice resulted in the significant reduction of urinary urate from day 1 of the treatment and was sustained during the study course of 6 days. Mean urine uric acid reduction was 43% (3.43±0.41 mg/24 h vs. 5.97±0.52 mg/24 h, FIG. 20).

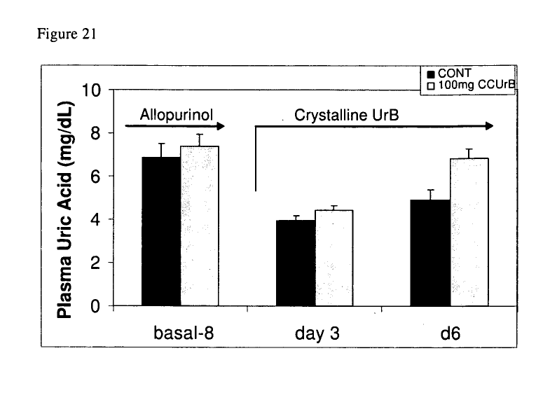

Despite the decrease in uric acid excretion, plasma urate was the same or slightly elevated when compared to placebo treated controls, FIG. 21. This data confirmed our previous observation, shown in FIG. 15 and FIG. 17, that could be only explained that the given dose of crystalline/coated uricase was not high enough to degrade excessive amounts of urate eliminated via entero-circulation.

### Conclusion

The results of initial studies in the Uox−/− mice with formulated uricase demonstrated hyperuricemia and hyperuricosuria reduction. High dose of uricase of 2000u/day normalized plasma and urine uric acid levels. However, lower doses of uricase (1000u/day or less) reduced excretion of uric acid, with no change in plasma urate.

Taken together, the results from all studies in Uox−/− mice shown in this example, are consistent with the notion that uricase, once protected from low pH in the gastric compartment, is able to degrade intraluminal urate that is eliminated from circulation via enteric excretion and consequently reduce the high concentration of circulating urate.

Based on these initial studies demonstrating efficacy, oral enzyme therapy with modified uricase has promising potential as a new oral agent for the treatment of hyperuricemia, hyperuricosuria, gout and related diseases.

### REFERENCES

1. Eustice C, Eustice R, Grossman K: “What is Hyperuricemia?” About.com. 2008 http://arthritis.about.com/od/gout/g/hyperuricemia.htm
2. Shiel WC: Gout and Hyperuricemia. MedicineNet.com. 2007 http://www.medicinenet.com/gout/article.htm
3. Coe F L, Strauss A L, Tembe V et al: Uric acid saturation and calcium nephrolithiasis. Kidney Int. 1980; 17:662-668
4. Marangella M: Uric Acid Elimination in the Urine. Pathophysiological implications. Contrib Nephrol. 2005; 147:132-48
5. Wu, X, et. al., Hyperuricemia and urate nephropathy in urate oxidase-deficient mice, Proc. Natl. Acad. Sci. USA, 1994, Vol. 91, pp. 742-746.

A number of embodiments of the invention have been described. Nevertheless, it will be understood that various modifications may be made without departing from the spirit and scope of the invention. Accordingly, other embodiments are within the scope of the following claims.

1. A composition comprising uricase and a pH increasing agent.

2. The composition of claim 1, wherein the composition further comprises a hydrogen peroxide degrading enzyme.

3. The composition of claim 2, wherein the hydrogen peroxide degrading enzyme comprises peroxidase or catalase.

4. The composition of claim 1, wherein the pH increasing agent comprises a compound selected from the group consisting of bicarbonate or a salt thereof, sodium bicarbonate, carbonate or a salt thereof, an anti acid and a proton pump inhibitor.

5-8. (canceled)

9. The composition of claim 1, wherein the uricase is stabilized by use of a polyionic reagent or a polyionic coating.

10. (canceled)

11. The composition of claim 9, wherein the polyionic coating is PSS: poly(Sodium 4-styrenesulfonate), PAA: polyacrylic acid sodium salt, PMG: poly(methylene-co-guanidine) hydrochloride, DS: dextran sulfate, PMA: poly(methylacrylate), or PVS: polyvinylsiloxane.

12. The composition of claim 1, wherein the uricase is crystalline.

13. A method of treating a disorder associated with elevated uric acid concentration in a subject, the method comprising: administering uricase and a pH increasing agent to a subject, wherein prior to administering the uricase and the pH increasing agent to the subject, the uric acid concentration in the subject is elevated as compared to a standard.

14. The method of claim 13, wherein the pH increasing agent increases pH to above about 5.

15-17. (canceled)

18. The method of claim 13, wherein the uric acid concentration is elevated in blood or urine.

19. (canceled)

20. The method of claim 13, further comprising lowering the uric acid concentration in the subject, wherein the lowering is compared to a standard.

21. The method of claim 13, wherein the pH increasing agent is selected from the group consisting of carbonate or a salt thereof, bicarbonate or a salt thereof, an anti-acid and a proton pump inhibitor.

22-37. (canceled)

38. A pharmaceutical composition comprising stabilized uricase.

39. (canceled)

40. The composition of claim 38, wherein the composition further comprises a hydrogen peroxide degrading enzyme.

41. The composition of claim 40, wherein the hydrogen peroxide degrading enzyme comprises peroxidase or catalase.

42. The composition of claim 38, wherein the uricase is stabilized by use of a polyionic reagent or a polyionic coating.

43. (canceled)

44. The composition of claim 42, wherein the polyionic coating is PSS: poly(Sodium 4-styrenesulfonate), PAA: poly Acrylic acid sodium salt, PMG: poly(methylene-co-guanidine) hydrochloride, DS: dextran sulfate, PMA: poly(methylacrylate), or PVS: Pol yvinylsiloxane.

45. The composition of claim 38, wherein the uricase is crystalline.

46. A method of treating a disorder associated with elevated uric acid concentration in a subject, the method comprising: administering stabilized uricase to a subject, wherein prior to administering the stabilized uricase to the subject, the uric acid concentration in the subject is elevated as compared to a standard.

47. The method of claim 46 further comprising administering a hydrogen peroxide degrading enzyme to the subject.

48-57. (canceled)
